(19) United States
(12) Patent Application Publication
(10) Pub. No.: US 2024/0298539 A1
(43) Pub. Date: Sep. 5, 2024

(54) ORGANIC ELECTROLUMINESCENT ELEMENT

(71) Applicant: NIPPON STEEL Chemical & Material Co., Ltd., Tokyo (JP)

(72) Inventors: Kentaro HAYASHI, Tokyo (JP); Yuta SAGARA, Tokyo (JP); Junya OGAWA, Tokyo (JP); Yuji IKENAGA, Tokyo (JP); Munetomo INOUE, Tokyo (JP); Satoshi UKIGAI, Tokyo (JP); Sayuri KITERA, Tokyo (JP); Tetsuro YAMASHITA, Tokyo (JP); Masataka OKUYAMA, Tokyo (JP); Mitsuru SAKAI, Tokyo (JP)

(73) Assignee: NIPPON STEEL Chemical & Material Co., Ltd., Tokyo (JP)

(21) Appl. No.: 18/577,273

(22) PCT Filed: Jul. 27, 2022
(86) PCT No.: PCT/JP2022/029026

§ 371 Date: Jan. 8, 2024

(30) Foreign Application Priority Data
Jul. 30, 2021 (JP) 2021-125937

(51) Int. Cl. H10K 85/60; C09K 11/06; H10K 50/12

(52) U.S. Cl. CPC H10K 85/6572; C09K 11/06; H10K 50/12; H10K 85/654; C09K 2211/1074

(57) ABSTRACT

To provide an organic EL device having a low voltage, high efficiency and extended lifetime characteristics, and a host material for use in the organic EL device. A host material for an organic EL device, including a compound represented by the following general formula (1), or a structural isomer thereof: wherein X represents N or C—H and at least one thereof represents N, L independently represents an aromatic hydrocarbon group, and R² to R⁶ represent hydrogen, an aliphatic hydrocarbon group, an aromatic hydrocarbon group, an aromatic heterocyclic group, or a linked aromatic group in which two to five of these aromatic rings are linked to each other, provided that R² and at least one of others do not represent hydrogen.

### TECHNICAL FIELD

The present invention relates to an organic electroluminescent element or device (hereinafter, referred to as an organic EL device), and specifically relates to an organic EL device comprising a specific mixed host material.

### BACKGROUND ART

Application of a voltage to an organic EL device allows injection of holes and electrons from an anode and a cathode, respectively, into a light-emitting layer. Then, in the light-emitting layer, injected holes and electrons recombine to generate excitons. At this time, according to statistical rules of electron spins, singlet excitons and triplet excitons are generated at a ratio of 1:3. Regarding a fluorescence-emitting organic EL device using light emission from singlet excitons, it is said that the internal quantum efficiency thereof has a limit of 25%. Meanwhile, regarding a phosphorescent organic EL device using light emission from triplet excitons, it is known that intersystem crossing is efficiently performed from singlet excitons, the internal quantum efficiency is enhanced to 100%.

Highly efficient organic EL devices utilizing delayed fluorescence have been developed recently. For example, Patent Literature 1 discloses an organic EL device utilizing a TTF (Triplet-Triplet Fusion) mechanism, which is one of delayed fluorescence mechanisms. The TTF mechanism utilizes a phenomenon in which singlet excitons are generated due to collision of two triplet excitons, and it is thought that the internal quantum efficiency can be theoretically raised to 40%. However, since the efficiency is lower compared to phosphorescent organic EL devices, further improvement in efficiency and low voltage characteristics are required.

In addition, patent Literature 2 discloses an organic EL device utilizing a TADF (Thermally Activated Delayed Fluorescence) mechanism. The TADF mechanism utilizes a phenomenon in which reverse intersystem crossing from triplet excitons to singlet excitons is generated in a material having a small energy difference between a singlet level and a triplet level, and it is thought that the internal quantum efficiency can be theoretically raised to 100%.

However, all the mechanisms have room for advancement in terms of both efficiency and lifetime, and are additionally required to be improved also in terms of reduction in driving voltage.

### CITATION LIST

### Patent Literature

Patent Literature 1: WO2010/134350 A
Patent Literature 2: WO2011/070963 A
Patent Literature 3: WO2008/056746 A
Patent Literature 4: WO2011/099374 A
Patent Literature 5: CN110776513 A
Patent Literature 6: KR2017-0056951 A
Patent Literature 7: WO2018/198844 A

Patent Literatures 3, 4, and 5 disclose use of an indolocarbazole compound as a host material of a light-emitting layer.

Patent Literature 6 discloses use of an indolocarbazole compound as a fluorescence-emitting material.

Patent Literature 7 discloses use of an indolocarbazole compound and a biscarbazole compound in a mixed host material of a light-emitting layer.

However, none of these can be said to be sufficient, and further improvement is desired.

### SUMMARY OF INVENTION

### Technical Problem

Organic EL displays, when compared with liquid crystal displays, are not only characterized by being thin-and-light, high in contrast, and capable of displaying a high-speed moving picture, but also highly valued in terms of designability such as curving and flexibility, and are widely applied in display apparatuses including mobiles and TV. However, organic EL displays are needed to be further reduced in voltage in order to suppress battery consumption in the case of use thereof for mobile terminals, and are inferior as light sources in terms of luminance and lifetime as compared with inorganic LEDs and thus are demanded to be improved in efficiency and stability during driving. In view of the above circumstances, an object of the present invention is to provide a practically useful organic EL device having a low voltage, high efficiency and lifetime characteristics.

As a result of intensive studies, the present inventors have found that the above problems can be solved by an organic EL device in which a specific host material is used in a light-emitting layer, and have completed the present invention.

The present invention relates to a host material for an organic EL device, represented by any of the following general formulas (1) to (5).

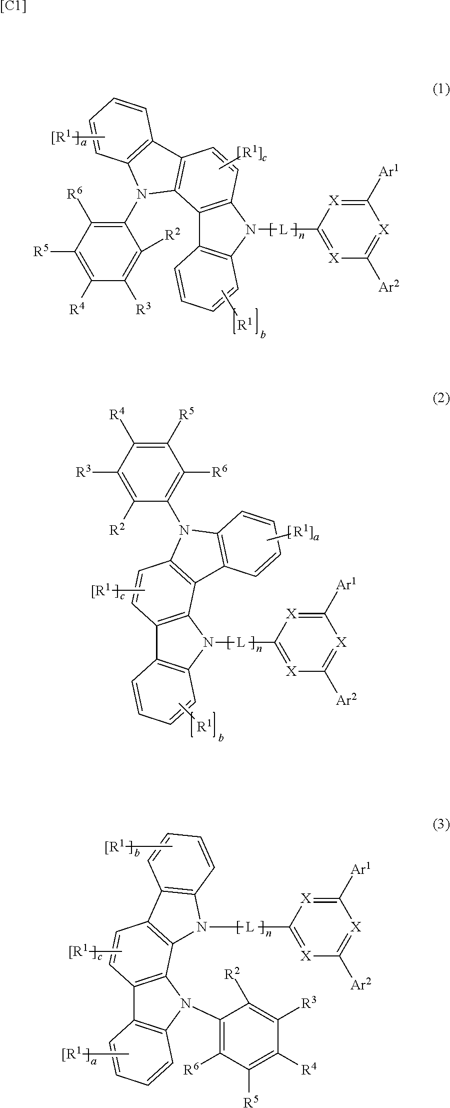

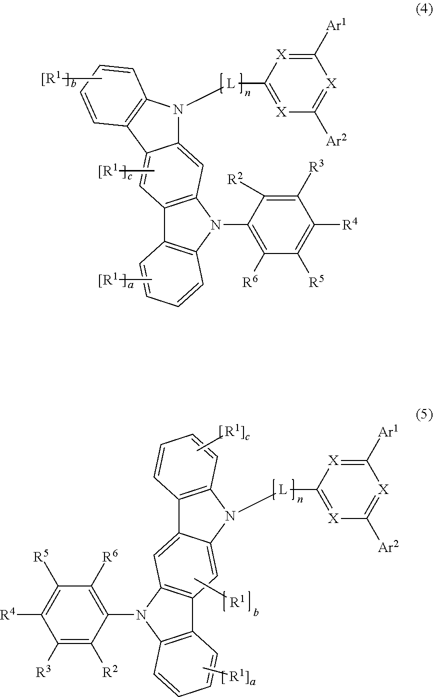

In the general formulas (1) to (5), each X independently represents N or C—H and at least one thereof represents N.

L independently represents a substituted or unsubstituted aromatic hydrocarbon group having 6 to 30 carbon atoms.

Ar¹ and Ar² each independently represent hydrogen, a substituted or unsubstituted aromatic hydrocarbon group having 6 to 30 carbon atoms, a substituted or unsubstituted aromatic heterocyclic group having 3 to 18 carbon atoms, or a substituted or unsubstituted linked aromatic group in which two to five aromatic rings of such an aromatic hydrocarbon group or aromatic heterocyclic group are linked to each other.

Each R¹ independently represents an aliphatic hydrocarbon group having 1 to 10 carbon atoms, a substituted or unsubstituted aromatic hydrocarbon group having 6 to 30 carbon atoms, or a substituted or unsubstituted aromatic heterocyclic group having 3 to 18 carbon atoms.

R² represents an aliphatic hydrocarbon group having 1 to 10 carbon atoms, a substituted or unsubstituted aromatic hydrocarbon group having 6 to 30 carbon atoms, a substituted or unsubstituted aromatic heterocyclic group having 3 to 18 carbon atoms, or a substituted or unsubstituted linked aromatic group in which two to five aromatic rings of such an aromatic hydrocarbon group or aromatic heterocyclic group are linked to each other.

R³ to R⁶ each independently represent hydrogen, an aliphatic hydrocarbon group having 1 to 10 carbon atoms, a substituted or unsubstituted aromatic hydrocarbon group having 6 to 30 carbon atoms, a substituted or unsubstituted aromatic heterocyclic group having 3 to 18 carbon atoms, or a substituted or unsubstituted linked aromatic group in which two to five aromatic rings of such an aromatic hydrocarbon group or aromatic heterocyclic group are linked to each other, and at least one of R³ to R⁶ represents a substituted or unsubstituted aromatic hydrocarbon group having 6 to 30 carbon atoms, or a substituted or unsubstituted aromatic heterocycle having 3 to 18 carbon atoms.

a to c represent the number of substitutions, a and b represent an integer of 0 to 4, and c represents an integer of 0 to 2. n represents the number of repetitions and an integer of 0 to 3.

In the general formulas (1) to (5), preferably, L represents a substituted or unsubstituted phenylene group and n represents 1 or 2, and more preferably, n represents 0.

Preferred aspects of the general formulas (1) to (5) include any of the following formulas (6) to (9).

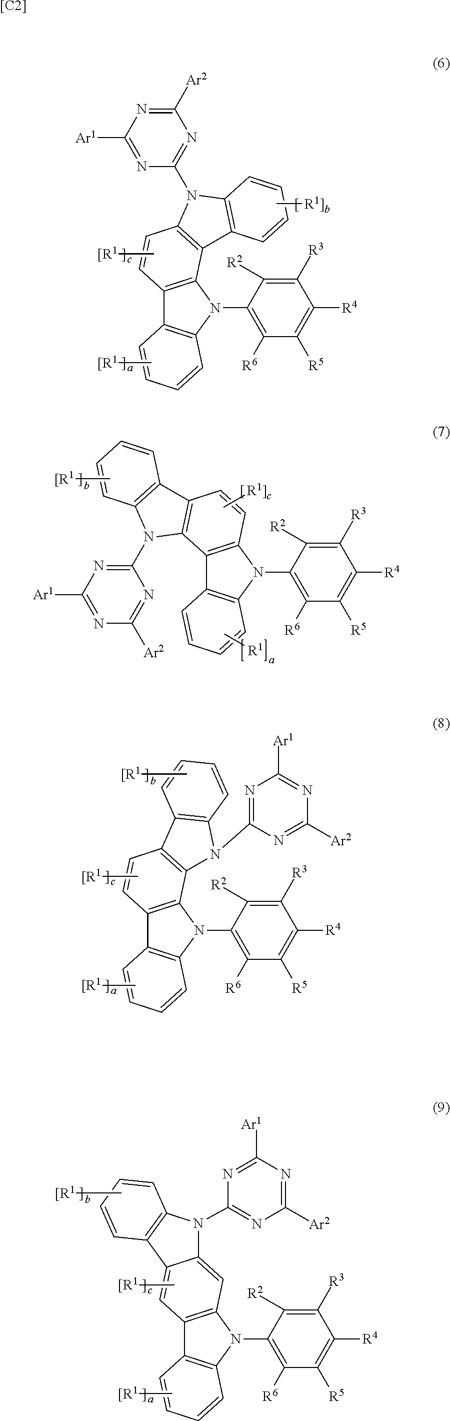

In the formulas (6) to (9), Ar¹, Ar², and a to c are as defined for the general formulas (1) to (5).

Each R¹ independently represents a substituted or unsubstituted aromatic hydrocarbon group having 6 to 18 carbon atoms, or a substituted or unsubstituted aromatic heterocyclic group having 3 to 12 carbon atoms.

R² represents a substituted or unsubstituted aromatic hydrocarbon group having 6 to 18 carbon atoms, a substituted or unsubstituted aromatic heterocyclic group having 3 to 12 carbon atoms, or a substituted or unsubstituted linked aromatic group in which two to five of these aromatic rings are linked to each other.

R³ to R⁶ each independently represent hydrogen, a substituted or unsubstituted aromatic hydrocarbon group having 6 to 18 carbon atoms, a substituted or unsubstituted aromatic heterocyclic group having 3 to 12 carbon atoms, or a substituted or unsubstituted linked aromatic group in which two to five aromatic rings of such an aromatic hydrocarbon group or aromatic heterocyclic group are linked to each other, and at least one of R³ to R⁶ represents a substituted or unsubstituted aromatic hydrocarbon group having 6 to 18 carbon atoms, or a substituted or unsubstituted aromatic heterocycle having 3 to 12 carbon atoms.

The present invention relates to an organic EL device comprising one or more light-emitting layers between an anode and a cathode opposed to each other, wherein at least one of the light-emitting layers contains a first host material selected from the host material according to any of the above, a second host material selected from a compound represented by the following general formula (10), and a light-emitting dopant material.

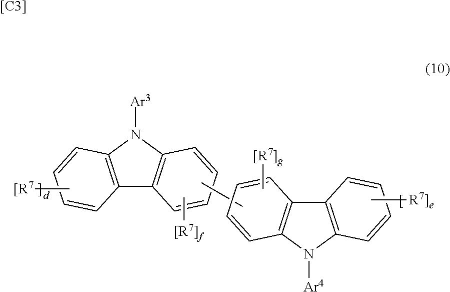

In the formula, Ar³ and Ar⁴ each independently represent a substituted or unsubstituted aromatic hydrocarbon group having 6 to 18 carbon atoms, a substituted or unsubstituted aromatic heterocyclic group having 3 to 17 carbon atoms, or a substituted or unsubstituted linked aromatic group in which two to five of these aromatic groups are linked to each other.

Each R⁷ independently represents deuterium, an aliphatic hydrocarbon group having 1 to 10 carbon atoms, a substituted or unsubstituted aromatic hydrocarbon group having 6 to 18 carbon atoms, or a substituted or unsubstituted aromatic heterocyclic group having 3 to 17 carbon atoms.

d to g represent the number of substitutions, d and e represent an integer of 0 to 4, and f and g represent an integer of 0 to 3.

In the general formula (10), Ar³ and Ar⁴ preferably each independently represent a substituted or unsubstituted phenyl group, a substituted or unsubstituted biphenyl group, or a substituted or unsubstituted terphenyl group.

Preferred examples of the first host material include a host material in which all of a to c in the general formulas (1) to (5) or formulas (6) to (9) represent 0, and preferred examples of the second host material include a host material in which all of d to g in the general formula (10) represent 0.

Examples of the light-emitting dopant material include an organic metal complex containing at least one metal selected from ruthenium, rhodium, palladium, silver, rhenium, osmium, iridium, platinum and gold, or a thermally activated delayed fluorescence-emitting dopant material.

The present invention also relates to a method for producing the organic EL device, comprising a step of mixing the first host material and the second host material in advance, and a step of vapor-depositing the resulting mixture from one vapor deposition source to form a light-emitting layer.

The present invention also relates to a composition comprising the first host material and the second host material.

Preferred examples of the first host material include a host material in which all of a to c in the general formulas (1) to (5) or formulas (6) to (9) represent 0, and preferred examples of the second host material include a host material in which all of d to g in the general formula (10) represent 0.

In preferred aspects, a difference in temperature at 50% weight loss of the first host material and the second host material is within 20° C.

### Advantageous Effects of Invention

The indolocarbazole compound in the present invention serves as a host material of a light-emitting layer to exhibit excellent characteristics. This compound and a biscarbazole compound can be mixed and used to thereby obtain an organic EL device exhibiting excellent characteristics.

### BRIEF DESCRIPTION OF DRAWING

FIG. 1 is a schematic cross-sectional view showing one example of an organic EL device.

### DESCRIPTION OF EMBODIMENTS

The host material for an organic EL device of the present invention is represented by any of the general formulas (1) to (5).

In the general formulas (1) to (5), each X independently represents N or C—H and at least one thereof represents N. Preferably, two or more X represent N. More preferably, all of X represent N.

L independently represents a substituted or unsubstituted aromatic hydrocarbon group having 6 to 30 carbon atoms. Preferred is a substituted or unsubstituted aromatic hydrocarbon group having 6 to 10 carbon atoms, and more preferred is a substituted or unsubstituted phenylene group. Specific examples of the unsubstituted aromatic hydrocarbon group having 6 to 30 carbon atoms are the same as those with respect to R² and R³ to R⁶ described below.

Ar¹ and Ar² each independently represent hydrogen, a substituted or unsubstituted aromatic hydrocarbon group having 6 to 30 carbon atoms, a substituted or unsubstituted aromatic heterocyclic group having 3 to 18 carbon atoms, or a substituted or unsubstituted linked aromatic group in which two to five aromatic rings of such an aromatic hydrocarbon group or aromatic heterocyclic group are linked to each other. Preferred is a substituted or unsubstituted aromatic hydrocarbon group having 6 to 18 carbon atoms, a substituted or unsubstituted aromatic heterocyclic group having 3 to 18 carbon atoms, or a substituted or unsubstituted linked aromatic group in which two to five of these aromatic rings are linked to each other, and more preferred is a substituted or unsubstituted aromatic hydrocarbon group having 6 to 18 carbon atoms, a substituted or unsubstituted aromatic heterocyclic group having 3 to 12 carbon atoms, or a substituted or unsubstituted linked aromatic group in which two to three of these aromatic rings are linked to each other. Specific examples of the unsubstituted aromatic hydrocarbon group having 6 to 30 carbon atoms, the unsubstituted aromatic heterocyclic group having 3 to 18 carbon atoms, or the linked aromatic group in which two to five of these aromatic rings are linked to each other are the same as those with respect to R² and R³ to R⁶ described below. Preferred examples thereof include a group generated from benzene, naphthalene, phenanthrene, fluorene, triphenylene, pyrene, carbazole, dibenzofuran, dibenzothiophene, indolocarbazole, benzofurocarbazole, benzothienocarbazole, pyridine, pyrimidine, triazine, or compounds in which two to five of these aromatic rings are linked to each other. More preferred is a phenyl group, a biphenyl group, a terphenyl group, a dibenzofuranyl group, or a dibenzothiophenyl group. The biphenyl group may be any of ortho-, meta-, or para-bonding. The terphenyl group may be linked linearly or branched.

In the present specification, the linked aromatic group refers to an aromatic group in which the aromatic rings in two or more aromatic groups are linked to each other by a single bond. The aromatic group here means an aromatic hydrocarbon group or an aromatic heterocyclic group. The linked aromatic group may be linear or branched. The linkage position in linking of benzene rings may be any of the ortho-, meta-, and para-positions, and is preferably the para-position or the meta-position. The aromatic group to be linked may be an aromatic hydrocarbon group or an aromatic heterocyclic group, and the plurality of aromatic groups may be the same or different. The phrase “these aromatic rings” in the linked aromatic group in which two to five of these aromatic rings are linked to each other means aromatic rings of the previously-mentioned aromatic hydrocarbon group or aromatic heterocyclic group.

Each R¹ independently represents an aliphatic hydrocarbon group having 1 to 10 carbon atoms, a substituted or unsubstituted aromatic hydrocarbon group having 6 to 30 carbon atoms, or a substituted or unsubstituted aromatic heterocyclic group having 3 to 18 carbon atoms. Each R¹ preferably represents an aliphatic hydrocarbon group having 1 to 4 carbon atoms, a substituted or unsubstituted aromatic hydrocarbon group having 6 to 12 carbon atoms, or a substituted or unsubstituted aromatic heterocyclic group having 3 to 12 carbon atoms. Specific examples of the unsubstituted aromatic hydrocarbon group having 6 to 30 carbon atoms, or the unsubstituted aromatic heterocyclic group having 3 to 18 carbon atoms are the same as those with respect to R² and R³ to R⁶ described below.

R² represents an aliphatic hydrocarbon group having 1 to 10 carbon atoms, a substituted or unsubstituted aromatic hydrocarbon group having 6 to 30 carbon atoms, a substituted or unsubstituted aromatic heterocyclic group having 3 to 18 carbon atoms, or a substituted or unsubstituted linked aromatic group in which two to five aromatic rings of such an aromatic hydrocarbon group or aromatic heterocyclic group are linked to each other. R³ to R⁶ each independently represent hydrogen, an aliphatic hydrocarbon group having 1 to 10 carbon atoms, a substituted or unsubstituted aromatic hydrocarbon group having 6 to 30 carbon atoms, a substituted or unsubstituted aromatic heterocyclic group having 3 to 18 carbon atoms, or a substituted or unsubstituted linked aromatic group in which two to five aromatic rings of such an aromatic hydrocarbon group or aromatic heterocyclic group are linked to each other. In particular, at least one of R³ to R⁶ represents a group other than hydrogen. In other words, at least one of R³ to R⁶ represents a substituted or unsubstituted aromatic hydrocarbon group having 6 to 30 carbon atoms, or a substituted or unsubstituted aromatic heterocyclic group having 3 to 18 carbon atoms.

R² and at least one of R³ to R⁶ preferably each represent a substituted or unsubstituted aromatic hydrocarbon group having 6 to 18 carbon atoms, a substituted or unsubstituted aromatic heterocyclic group having 3 to 12 carbon atoms, or a substituted or unsubstituted linked aromatic group in which two to five of these aromatic rings are linked to each other. R² and at least one of R³ to R⁶ more preferably each represent a substituted or unsubstituted aromatic hydrocarbon group having 6 to 18 carbon atoms, a substituted or unsubstituted aromatic heterocyclic group having 3 to 12 carbon atoms, or a substituted or unsubstituted linked aromatic group in which two to three of these aromatic rings are linked to each other. A group other than at least one of R³ to R⁶ is preferably hydrogen.

Specific examples of the unsubstituted aromatic hydrocarbon group having 6 to 30 carbon atoms, the unsubstituted aromatic heterocyclic group having 3 to 18 carbon atoms, or the linked aromatic group in which two to five of these aromatic rings are linked to each other include a group generated by removing one hydrogen from benzene, naphthalene, acenaphthene, acenaphthylene, azulene, anthracene, chrysene, pyrene, phenanthrene, fluorene, triphenylene, pyridine, pyrimidine, triazine, thiophene, isothiazole, thiazole, pyridazine, pyrrole, pyrazole, imidazole, triazole, thiadiazole, pyrazine, furan, isoxazole, quinoline, isoquinoline, quinoxaline, quinazoline, thiadiazole, phthalazine, tetrazole, indole, benzofuran, benzothiophene, benzoxazole, benzothiazole, indazole, benzimidazole, benzotriazole, benzisothiazole, benzothiadiazole, purine, pyranone, coumarin, isocoumarin, chromone, dibenzofuran, dibenzothiophene, dibenzoselenophene, carbazole, indolocarbazole, benzofuranylcarbazole, benzothienocarbazole, or compounds in which two to five of these aromatic rings are linked to each other. Preferred examples thereof include a group generated from benzene, naphthalene, phenanthrene, fluorene, triphenylene, dibenzofuran, dibenzothiophene, pyridine, pyrimidine, triazine, or compounds in which two to five of these aromatic rings are linked to each other. More preferred is a phenyl group, a biphenyl group, or a terphenyl group. The biphenyl group may be any of ortho-, meta-, or para-bonding. The terphenyl group may be linked linearly or branched.

Specific examples of the aliphatic hydrocarbon group having 1 to 10 carbon atoms include methyl, ethyl, propyl, isopropyl, butyl, t-butyl, pentyl, neopentyl, hexyl, heptyl, octyl, nonyl, and decyl. Preferred is methyl, ethyl, t-butyl, or neopentyl, and more preferred is methyl.

a to c represent the number of substitutions, a and b represent an integer of 0 to 4, and c represents an integer of 0 to 2. Preferably, a and b represent an integer of 0 to 2, and c represents an integer of 0 to 1. More preferably, all of a, b, and c represent 0.

n represents the number of repetitions and an integer of 0 to 3, preferably represents 0 or 1, more preferably represents 0.

The compound represented by any of the general formulas (1) to (5) is more preferably represented by the formulas (6) to (9). In the formulas, the same symbols as those in the general formulas (1) to (5) have the same meaning. Herein, the general formula (1) corresponds to the formula (6), the general formula (2) corresponds to the formula (7), the general formula (3) corresponds to the formula (8), and the general formula (4) corresponds to the formula (9), and such formulas are understood as respective preferred aspects. The formulas (6) to (9) are understood as aspects where n in the general formulas (1) to (4) represents 0.

The host material is used as a host material of a light-emitting layer of an organic EL device. The host material may be adopted singly, and two or more kinds thereof are preferably adopted. When two or more kinds thereof are adopted, preferably, the host material is included as the first host material and a material selected from the compound represented by the general formula (10) is included as the second host material.

In the general formula (10), Ar³ and Ar⁴ each independently represent a substituted or unsubstituted aromatic hydrocarbon group having 6 to 18 carbon atoms, a substituted or unsubstituted aromatic heterocyclic group having 3 to 17 carbon atoms, or a substituted or unsubstituted linked aromatic group in which two to five aromatic rings of these aromatic groups are linked to each other. Preferred is a substituted or unsubstituted phenyl group, a substituted or unsubstituted aromatic heterocyclic group having 3 to 12 carbon atoms, or a substituted or unsubstituted linked aromatic group in which two to five of these aromatic rings are linked to each other, and more preferred is a substituted or unsubstituted phenyl group, a substituted or unsubstituted biphenyl group, a substituted or unsubstituted terphenyl group, or a substituted or unsubstituted aromatic heterocyclic group having 3 to 12 carbon atoms. The biphenyl group may be any of ortho-, meta-, or para-bonding. The terphenyl group may be linked linearly or branched.

Each R⁷ independently represents an aliphatic hydrocarbon group having 1 to 10 carbon atoms, a substituted or unsubstituted aromatic hydrocarbon group having 6 to 18 carbon atoms, or a substituted or unsubstituted aromatic heterocyclic group having 3 to 17 carbon atoms. Each R⁷ preferably represents a substituted or unsubstituted phenyl group, or a substituted or unsubstituted aromatic heterocyclic group having 3 to 12 carbon atoms. More preferred is a substituted or unsubstituted phenyl group, or a substituted or unsubstituted aromatic heterocyclic group having 3 to 12 carbon atoms.

Specific examples of the unsubstituted aromatic hydrocarbon group having 6 to 18 carbon atoms, the unsubstituted aromatic heterocyclic group having 3 to 17 carbon atoms, or the linked aromatic group in which two to five of these aromatic rings are linked to each other are understood from specific examples of a case where R² to R⁶ represent an unsubstituted aromatic hydrocarbon group, an aromatic heterocyclic group, or a linked aromatic group. Herein, a case where the number of carbon atoms does not fall within the above range is excluded. Specific examples of the aliphatic hydrocarbon group having 1 to 10 carbon atoms are the same as those described with respect to R¹ to R⁶.

d to g represent the number of substitutions, d and e represent an integer of 0 to 4, and f and g represent an integer of 0 to 3. Preferably, d and e represent an integer of 0 to 2, and f and g represent 0 or 1. More preferably, all of d, e, f, and g represent 0.

The form represented by the general formula (10) is preferably biscarbazole in which the substitution position of at least one carbazole is the 3-position, and more preferably 3,3′-biscarbazole.

In the present specification, for example, the aromatic hydrocarbon group, the aromatic heterocyclic group, or the linked aromatic group can have a substituent.

Specific examples of the substituent include cyano, methyl, ethyl, propyl, i-propyl, butyl, t-butyl, pentyl, neopentyl, cyclopentyl, hexyl, cyclohexyl, heptyl, octyl, nonyl, decyl, vinyl, propenyl, butenyl, pentenyl, methoxy, ethoxy, propoxy, butoxy, pentoxy, diphenylamino, naphthylphenylamino, dinaphthylamino, dinthranylamino, diphenanthrenylamino, and dipyrenylamino. Preferred examples thereof include cyano, methyl, ethyl, t-butyl, propyl, butyl, pentyl, neopentyl, hexyl, heptyl, octyl, diphenylamino, naphthylphenylamino, or dinaphthylamino.

In the present invention, some or all of hydrogen in the compound represented by the general formula (10) may be deuterium. A deuterated product encompasses both the case of a single compound included and the case of a mixture of two or more compounds included. In other words, the rate of deuteration is specifically described, and means that half the total hydrogen, on average, is substituted with deuterium in the case of a rate of deuteration of 50%, in which the deuterated product may be a single compound or a mixture of those different in rate of deuteration.

When some of hydrogen in the compound represented by the general formula (10) are deuterium, preferably 30% or more of hydrogen atoms are deuterium, more preferably 40% or more thereof are deuterium, and further preferably 50% or more thereof are deuterium.

The rate of deuteration can be determined by mass analysis or proton nuclear magnetic resonance spectroscopy. For example, when the rate of deuteration is determined by proton nuclear magnetic resonance spectroscopy, first, a measurement sample is prepared by adding and dissolving a compound and an internal standard material to and in a deuterated solvent, and the proton concentration [mol/g] in the compound included in the measurement sample is calculated from the ratio between the respective integral intensities derived from the internal standard material and the compound. Next, the ratio between the proton concentration in a deuterated compound and the proton concentration in the corresponding non-deuterated compound is calculated, and subtracted from 1, thereby enabling calculation of the rate of deuteration of the deuterated compound. The rate of deuteration of a partial structure can be calculated from the integral intensity of the chemical shift derived from an objective partial structure, according to the same procedure.

Some or all hydrogen atoms of the unsubstituted aromatic hydrocarbon group, the unsubstituted aromatic heterocyclic group, the unsubstituted linked aromatic group, the substituents of these aromatic groups, or the aliphatic hydrocarbon group may be deuterated. In other words, some or all of hydrogen on an aromatic ring in the general formula (10), and some or all of hydrogen in Ar³, Ar⁴, R⁷, and the like may be deuterium.

Specific examples of the compounds represented by the general formulas (1) to (5) are shown below, but are not limited to these exemplified compounds.

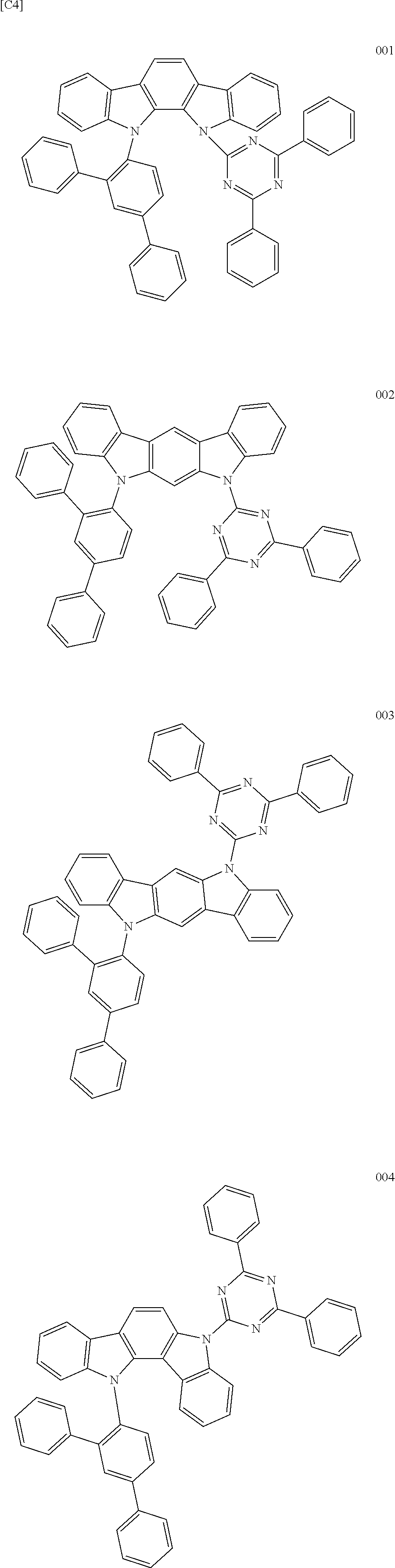

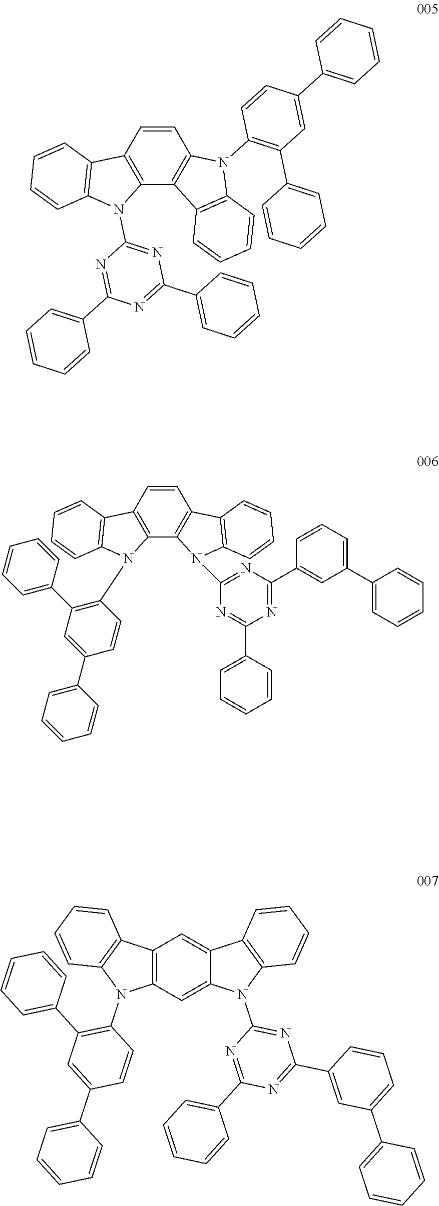

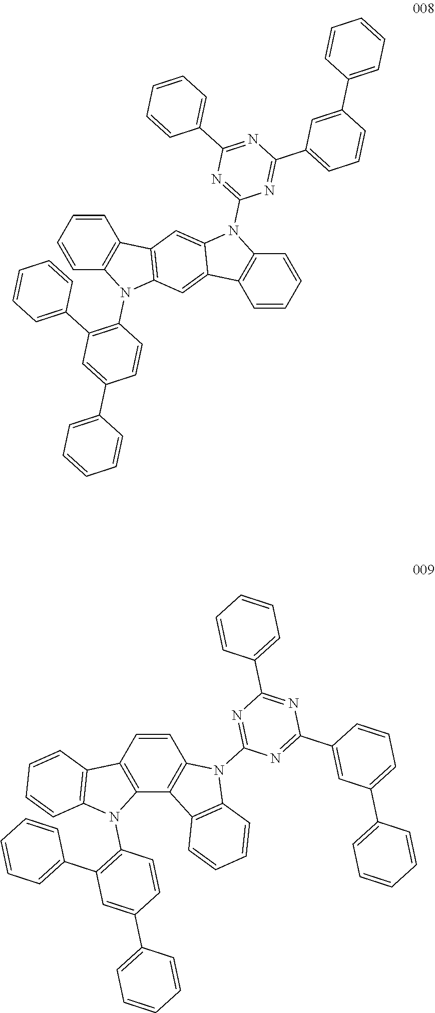

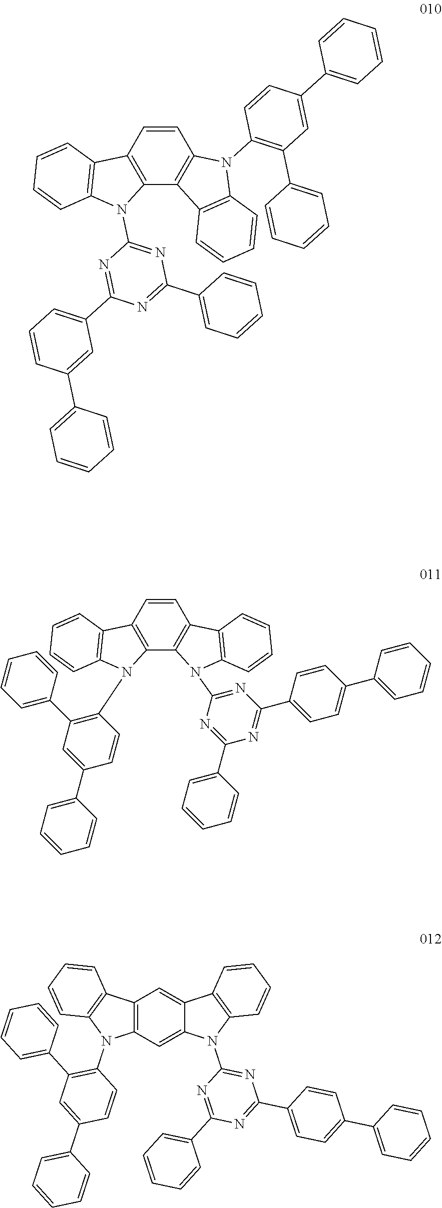

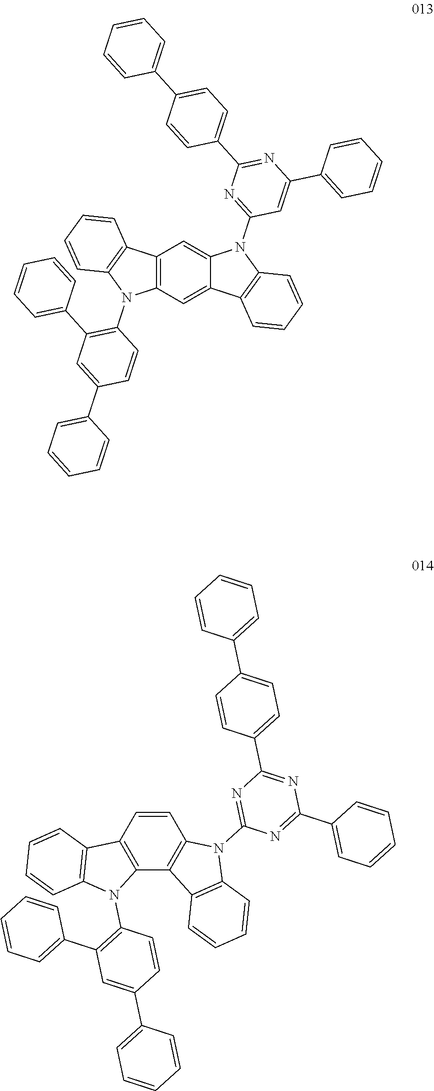

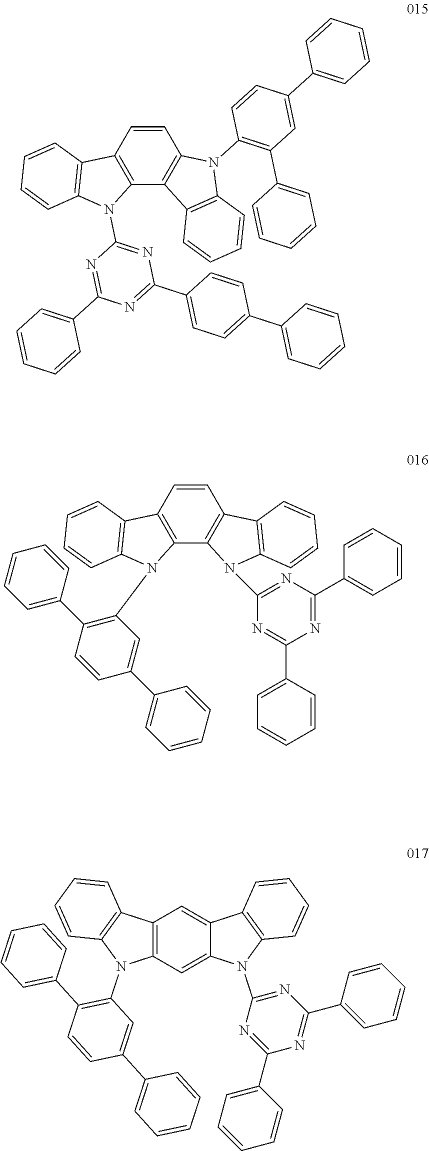

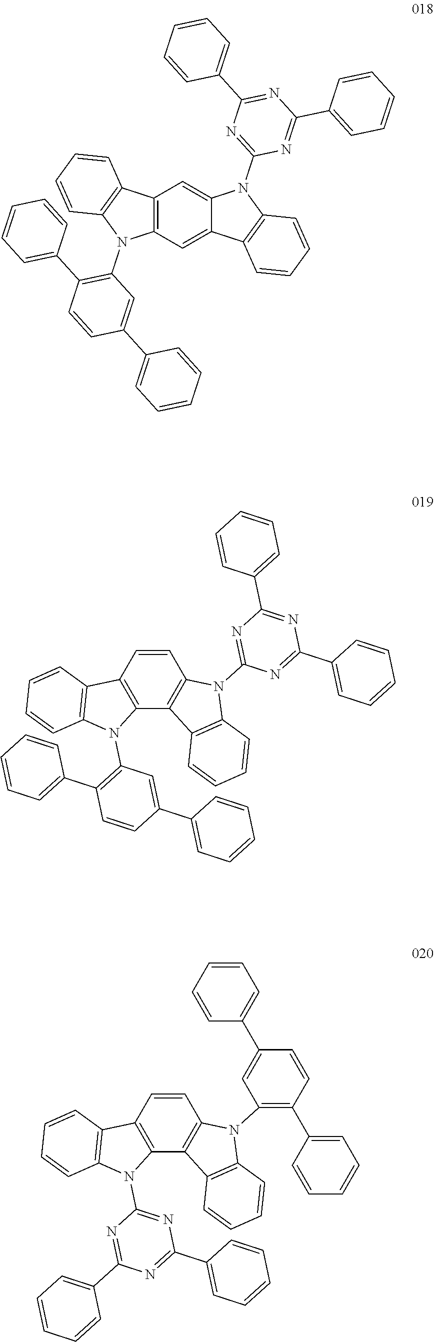

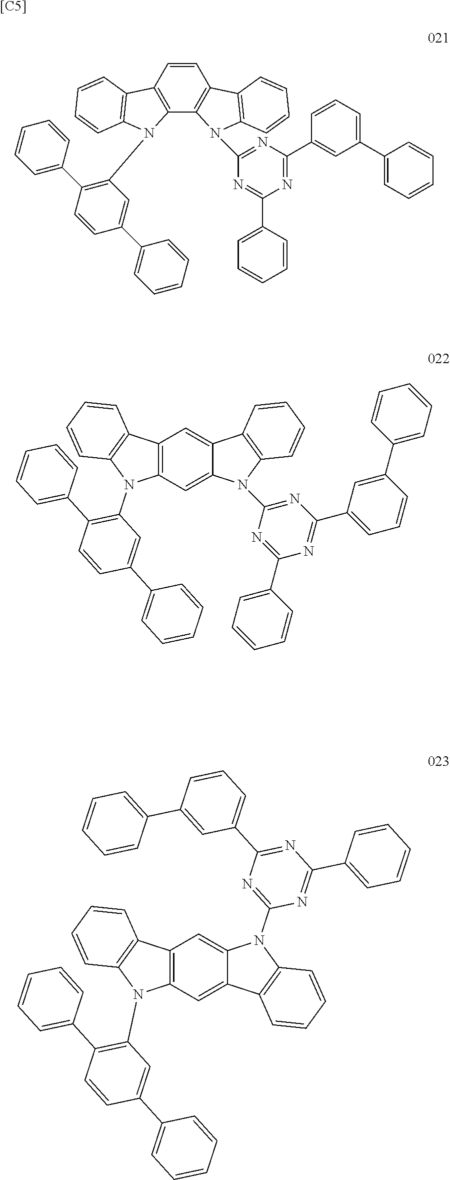

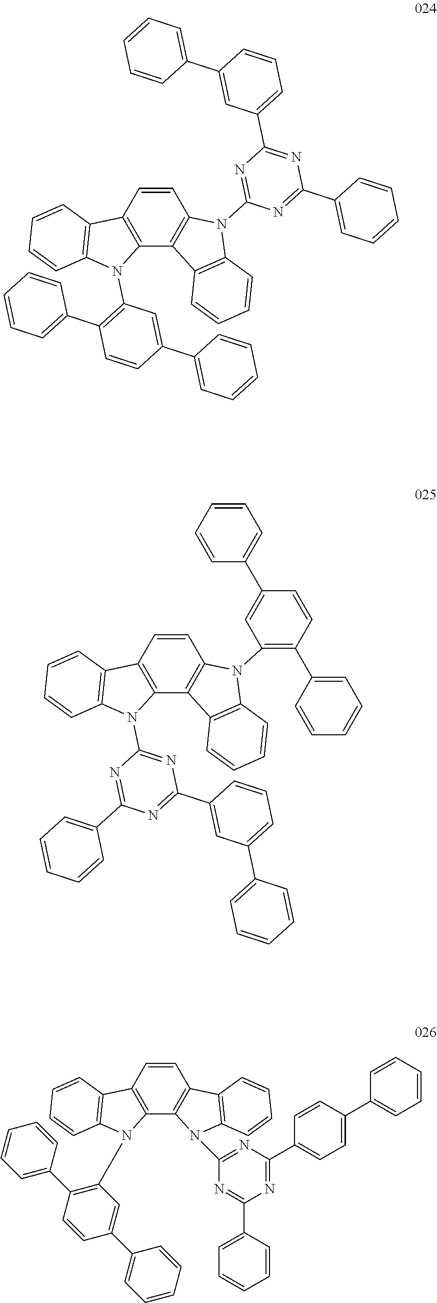

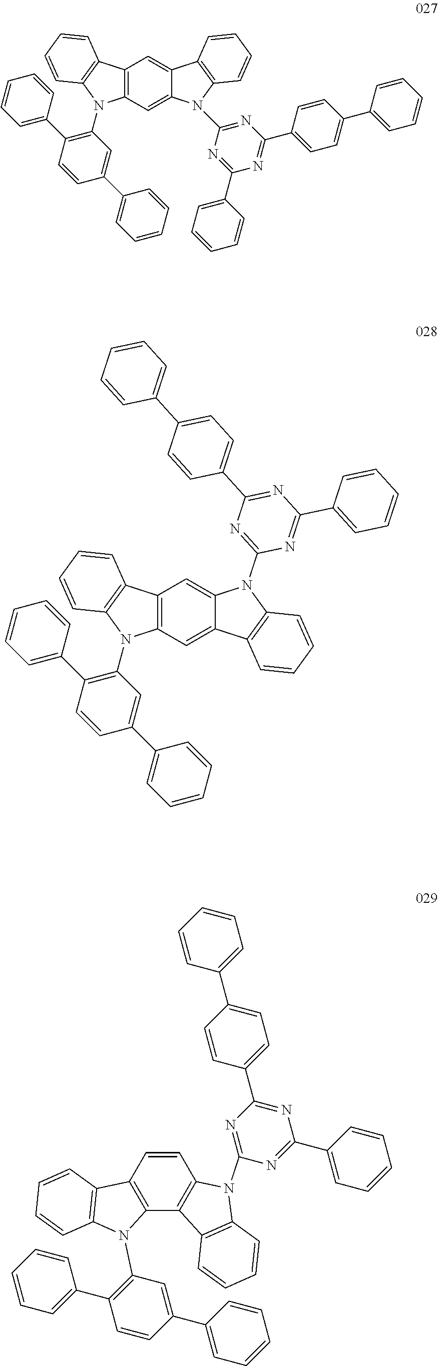

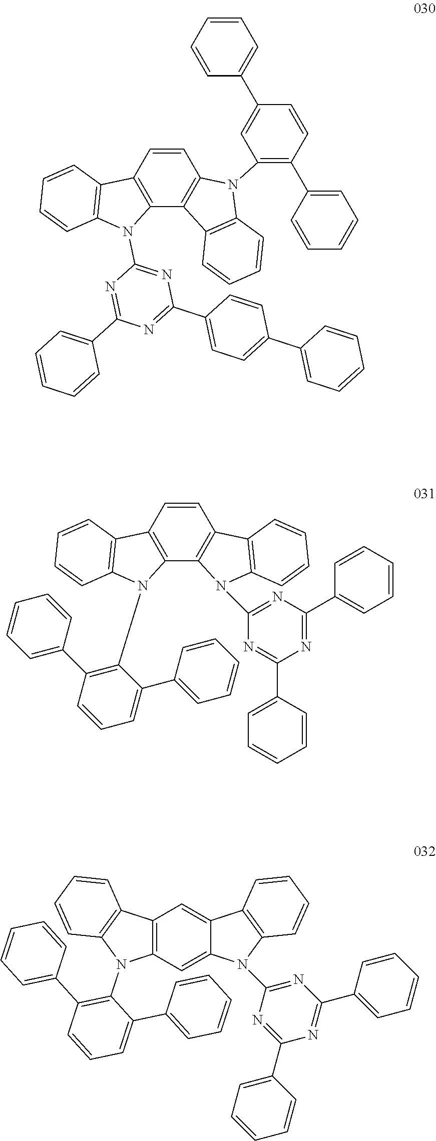

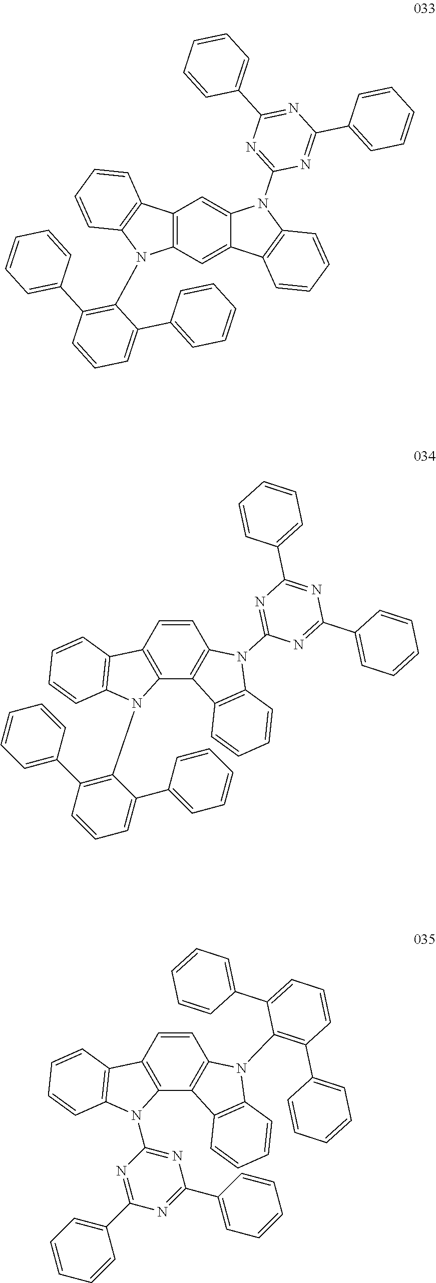

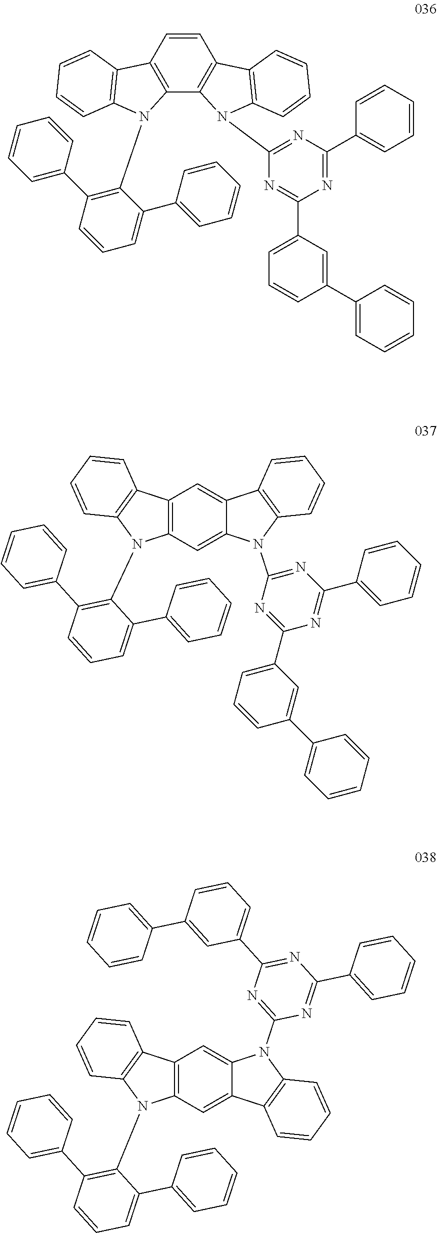

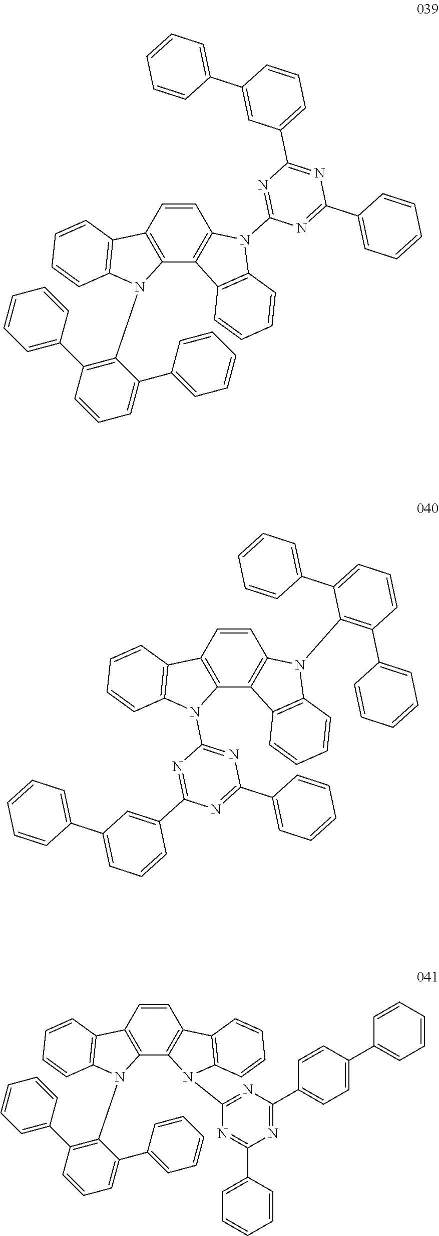

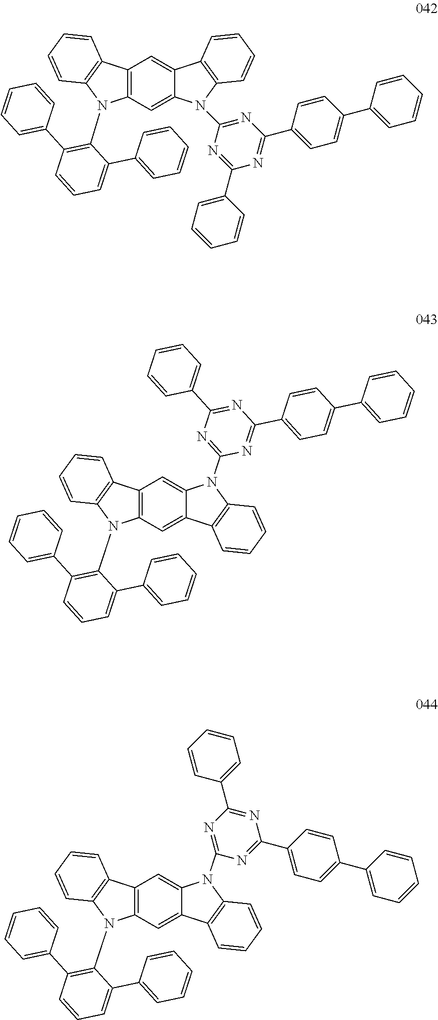

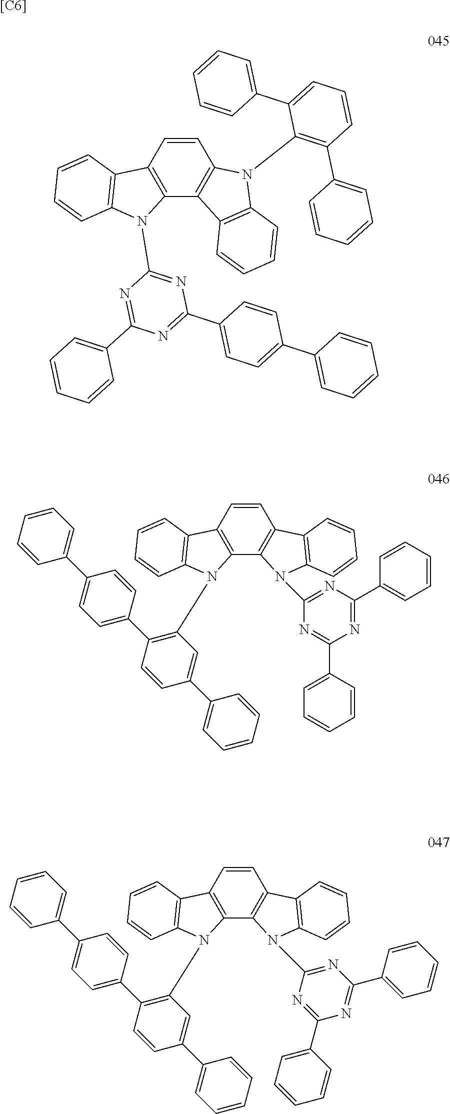

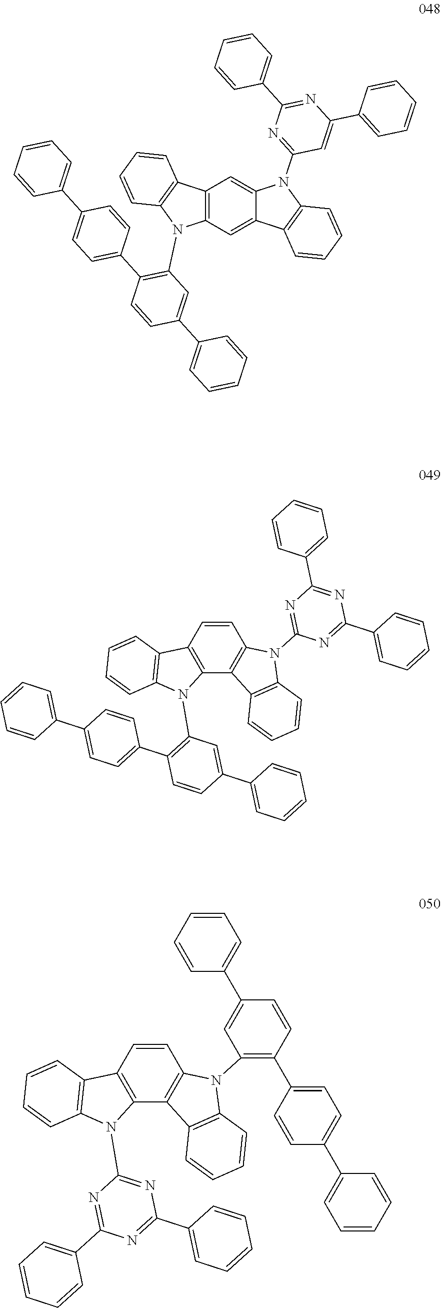

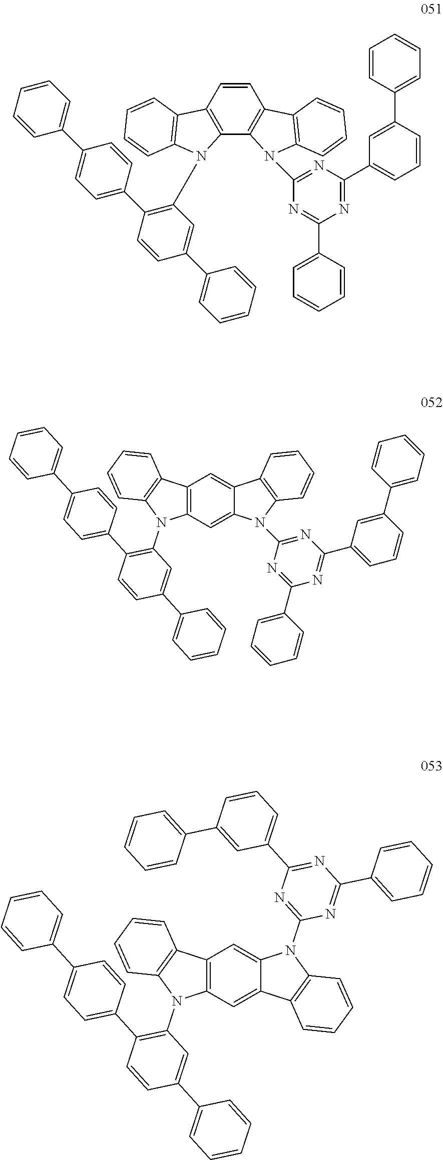

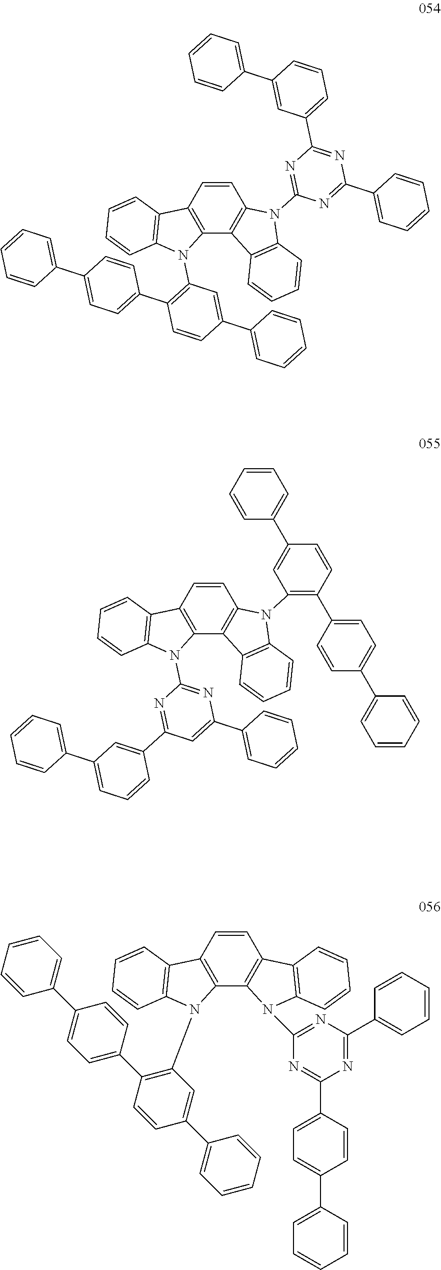

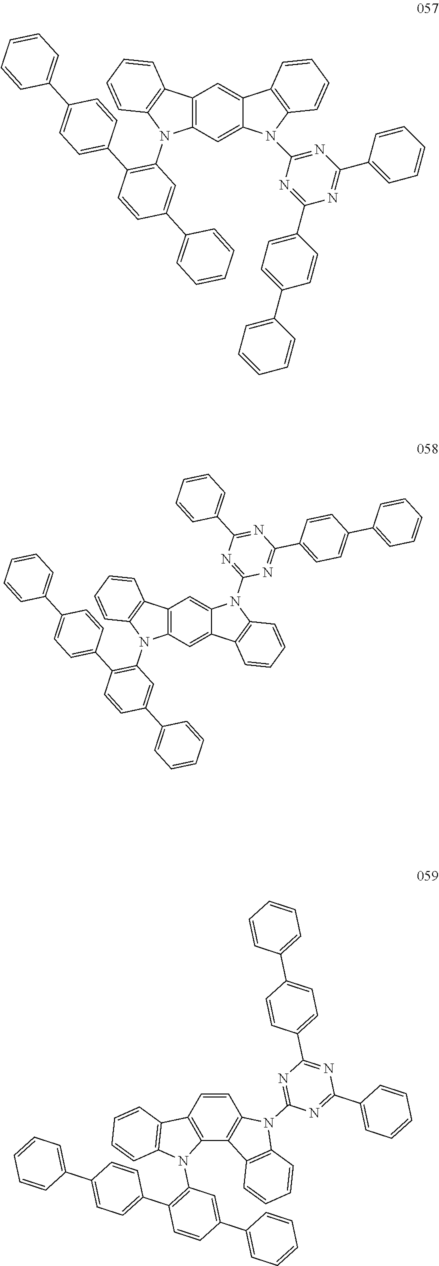

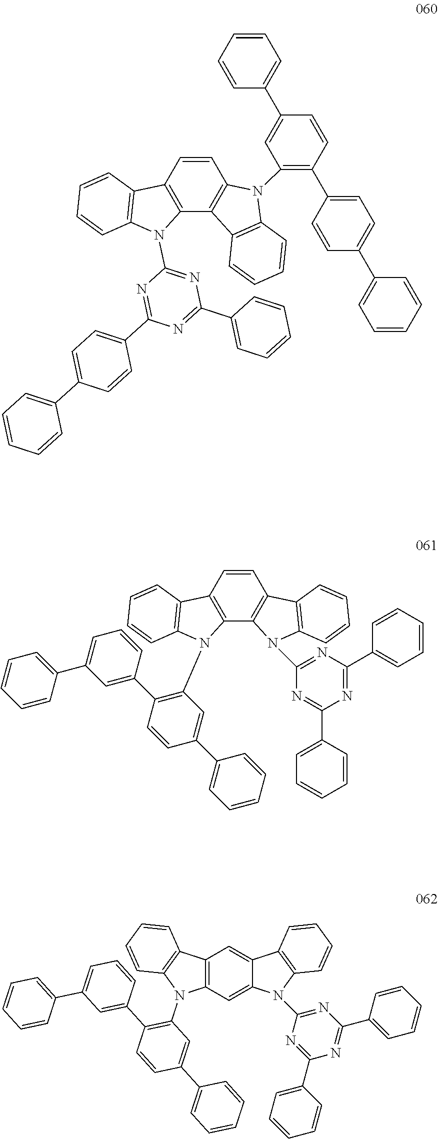

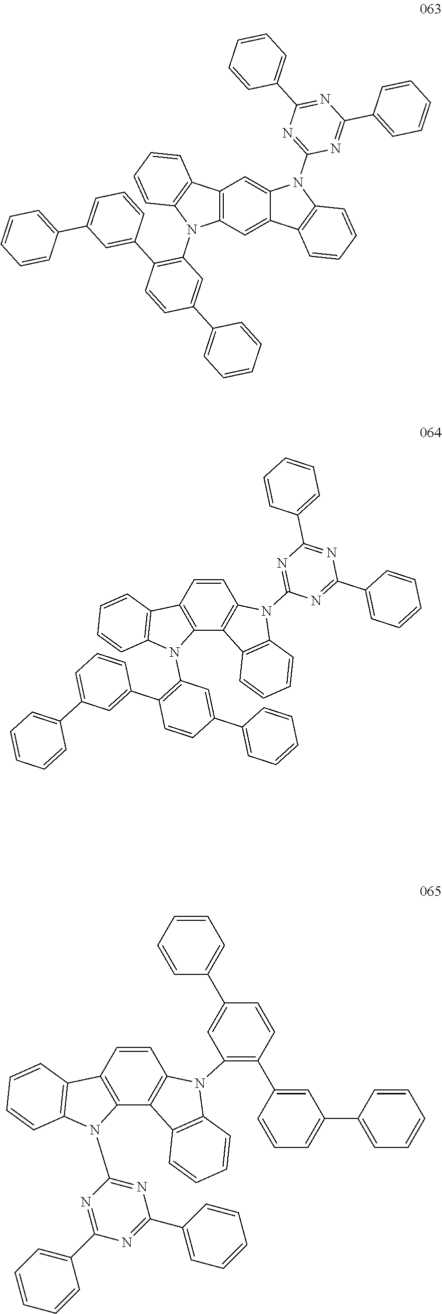

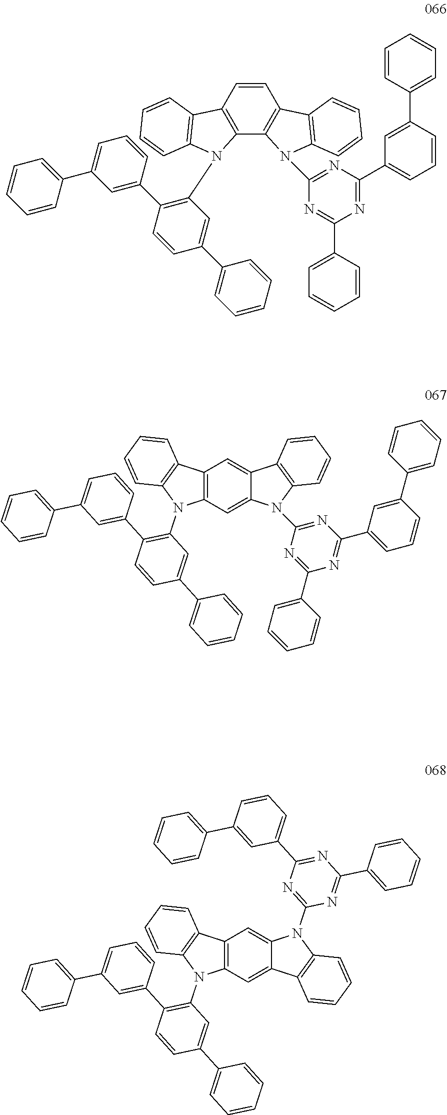

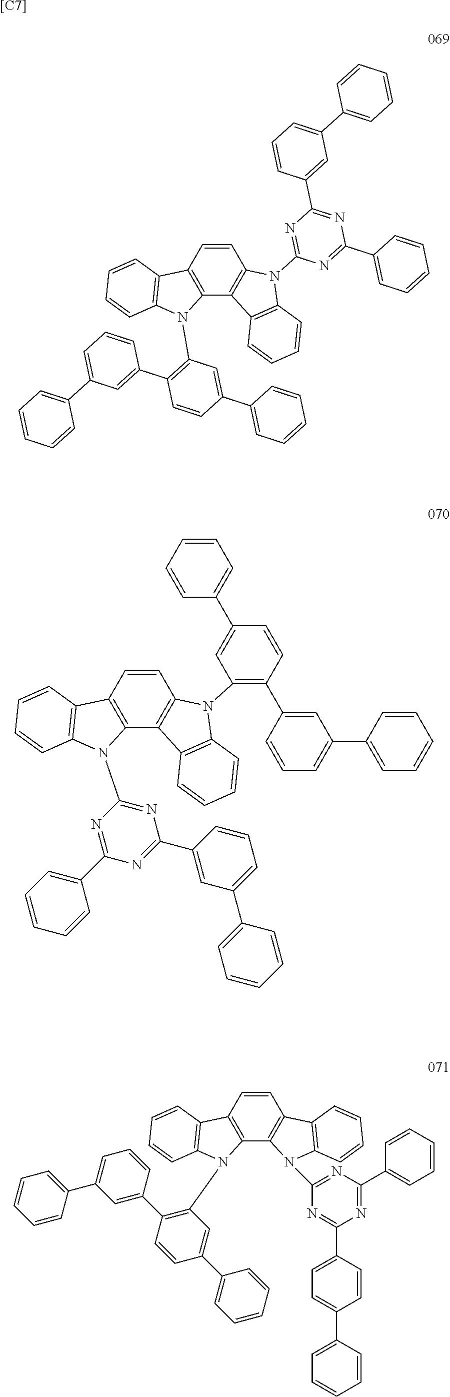

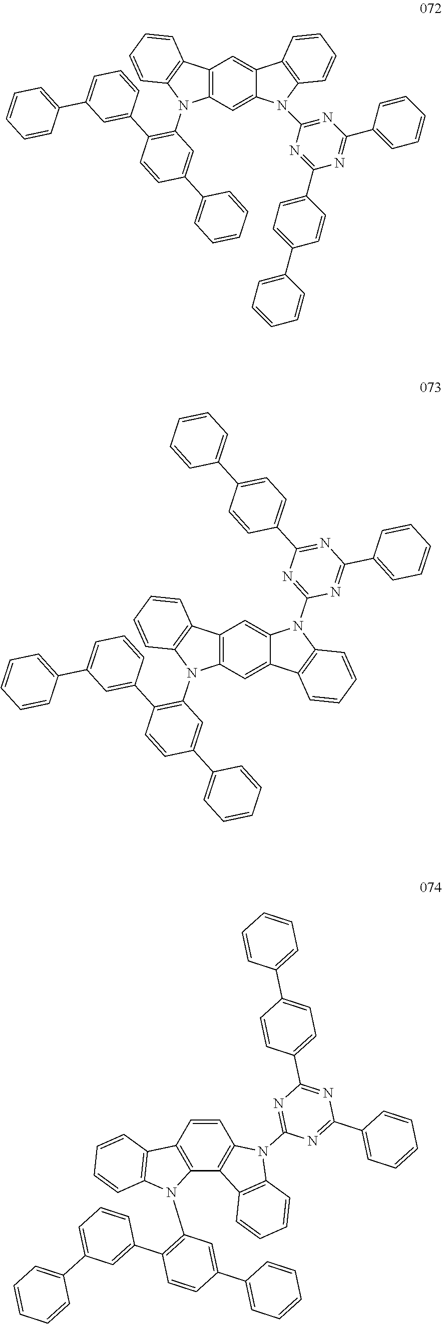

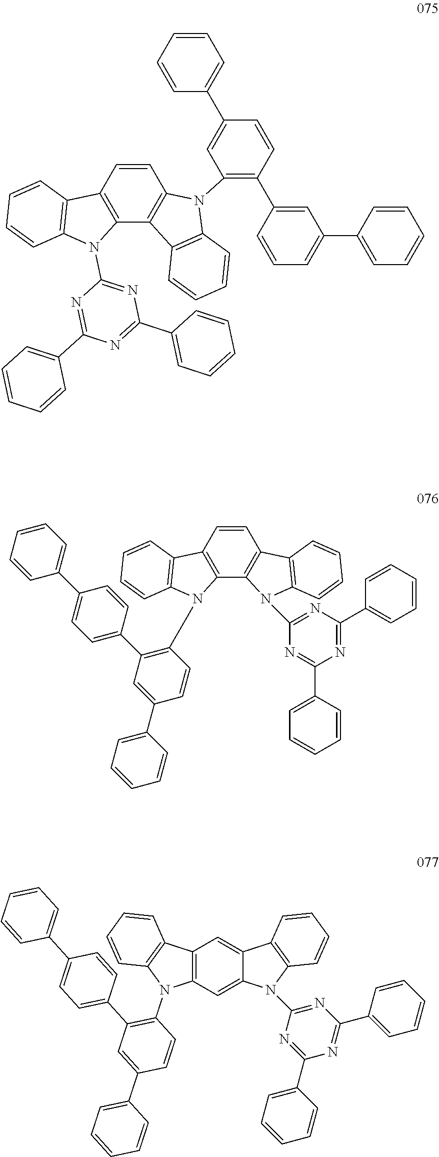

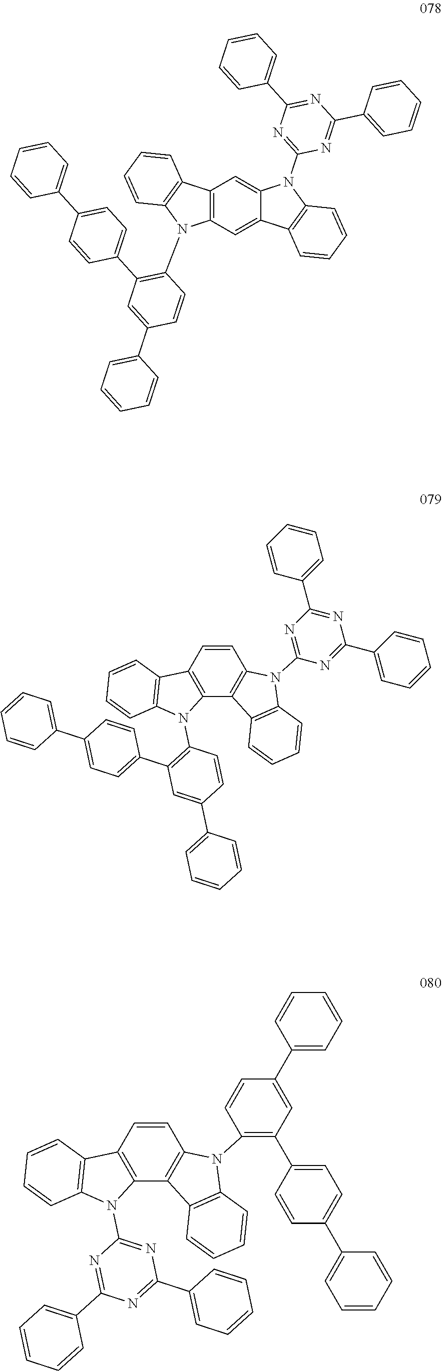

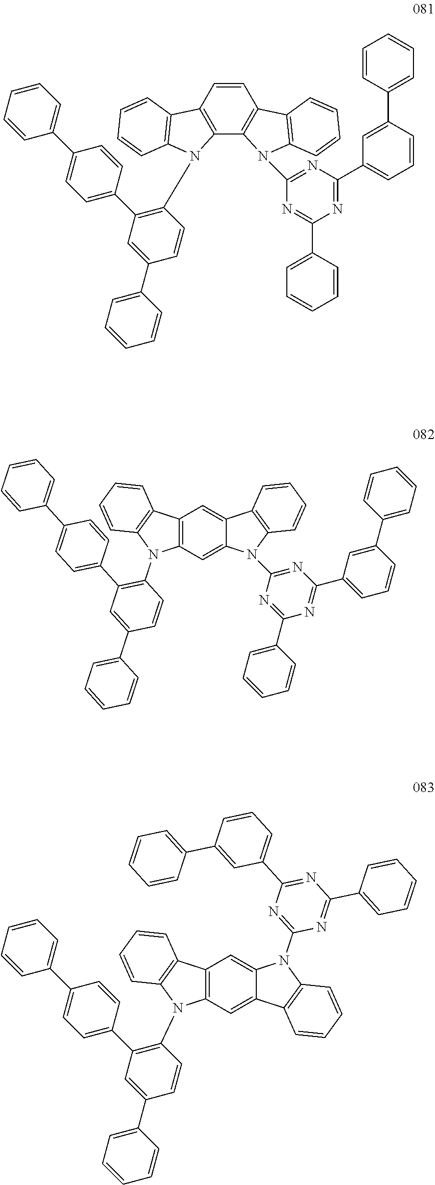

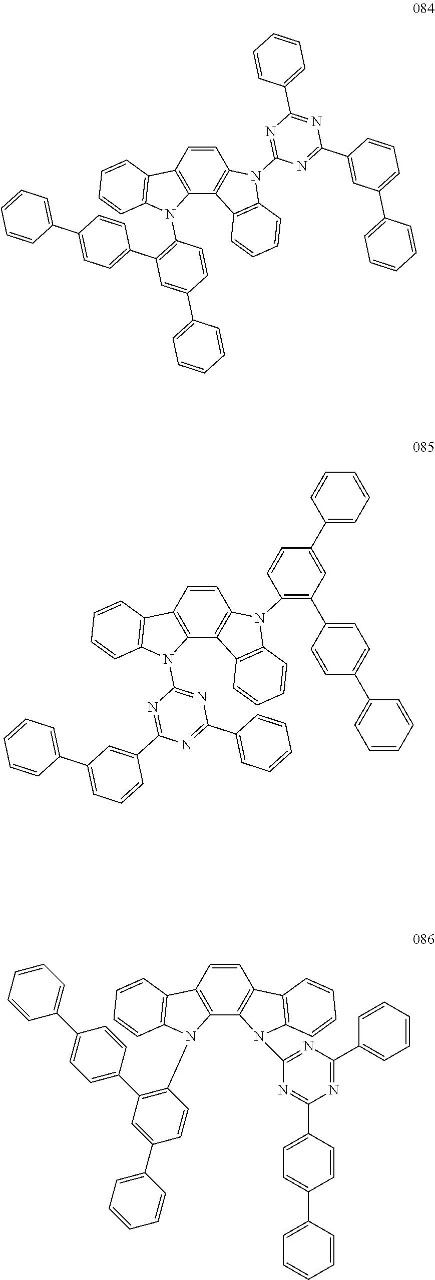

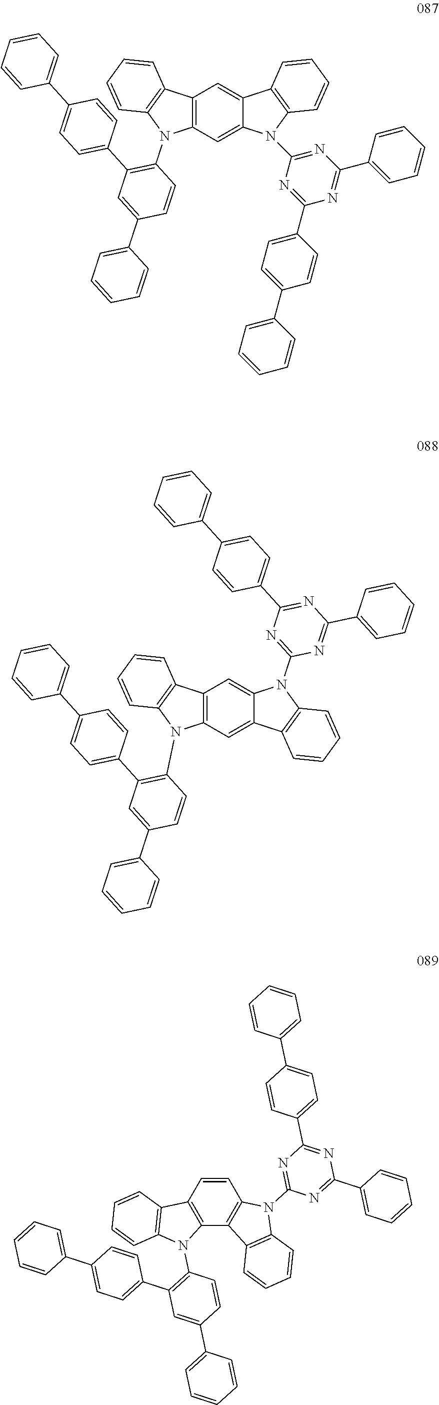

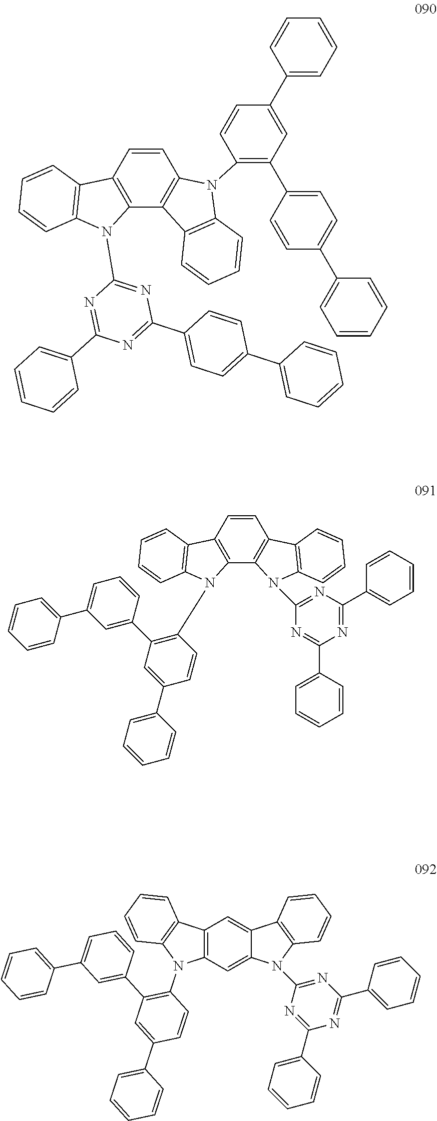

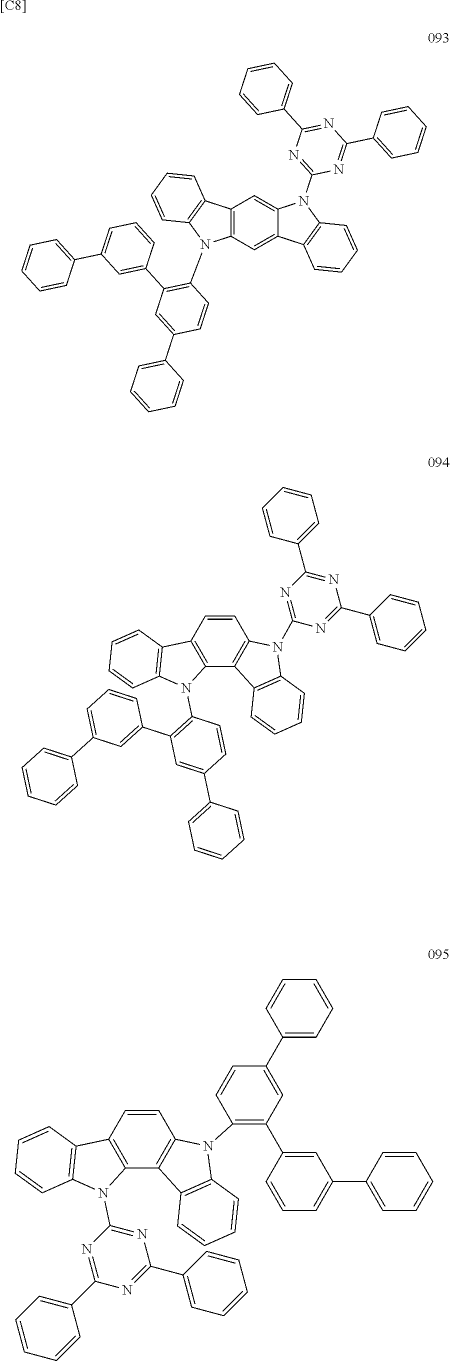

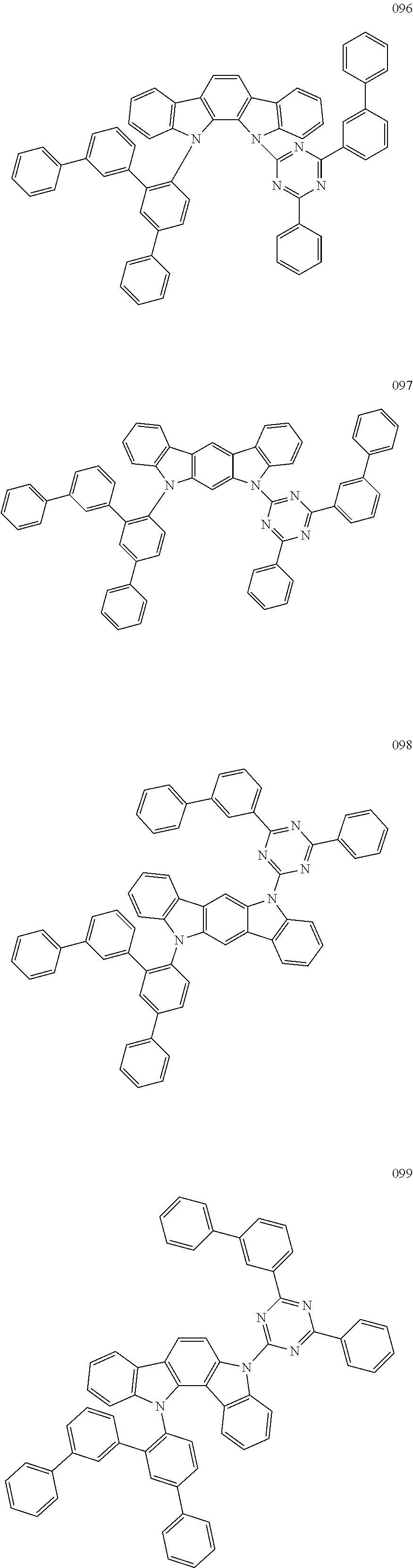

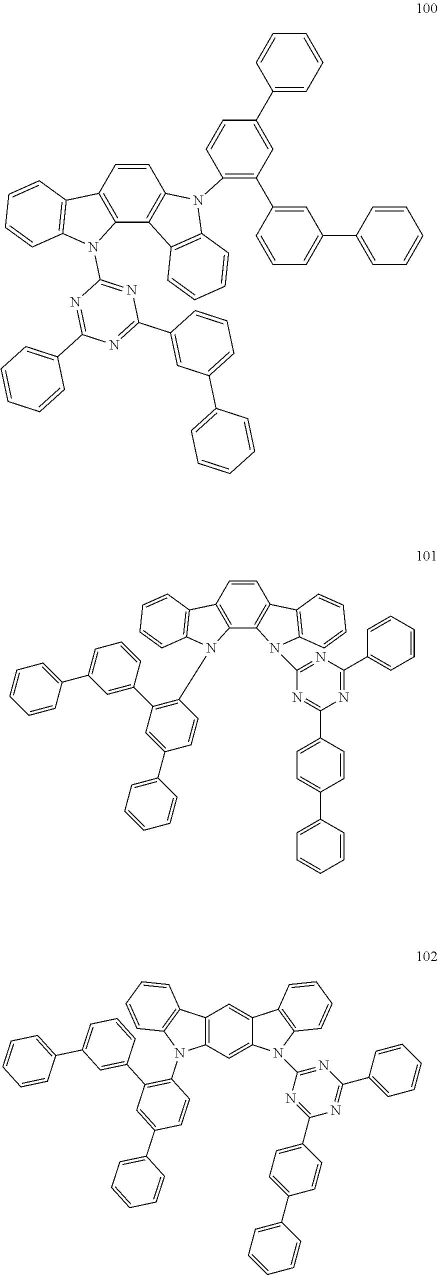

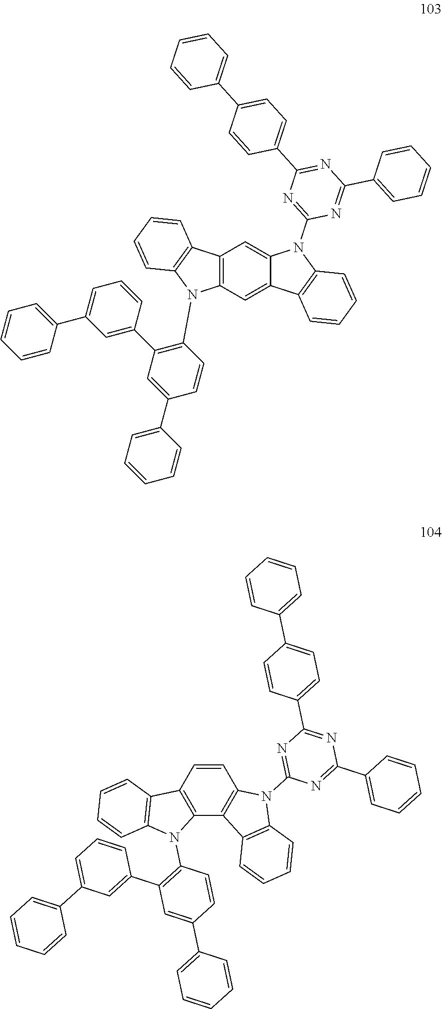

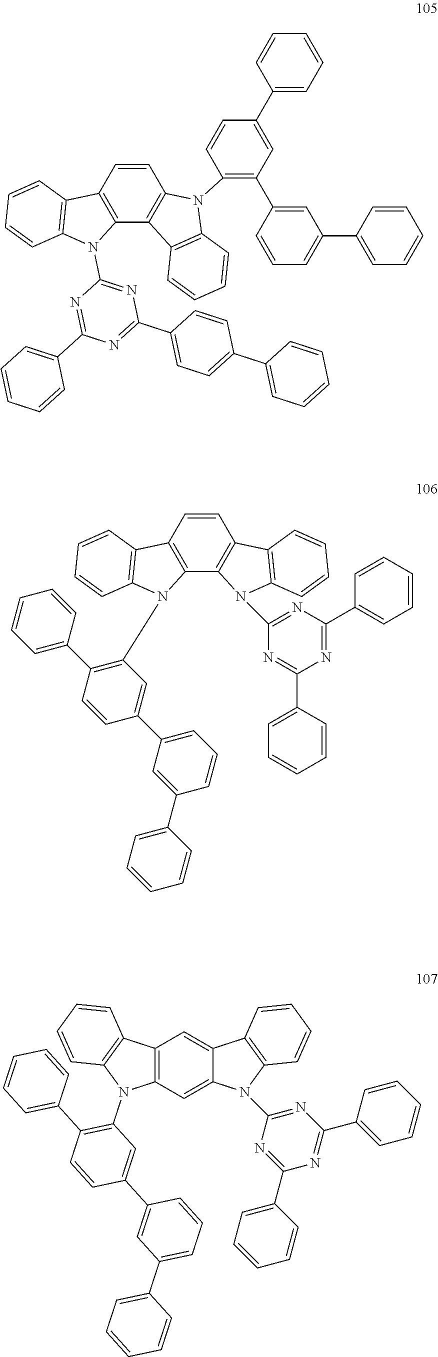

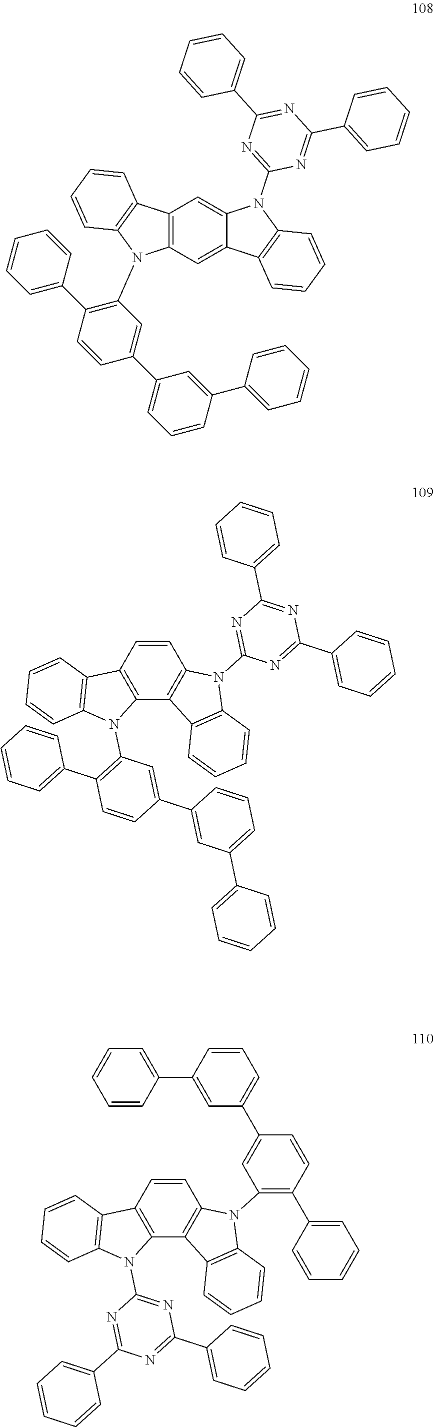

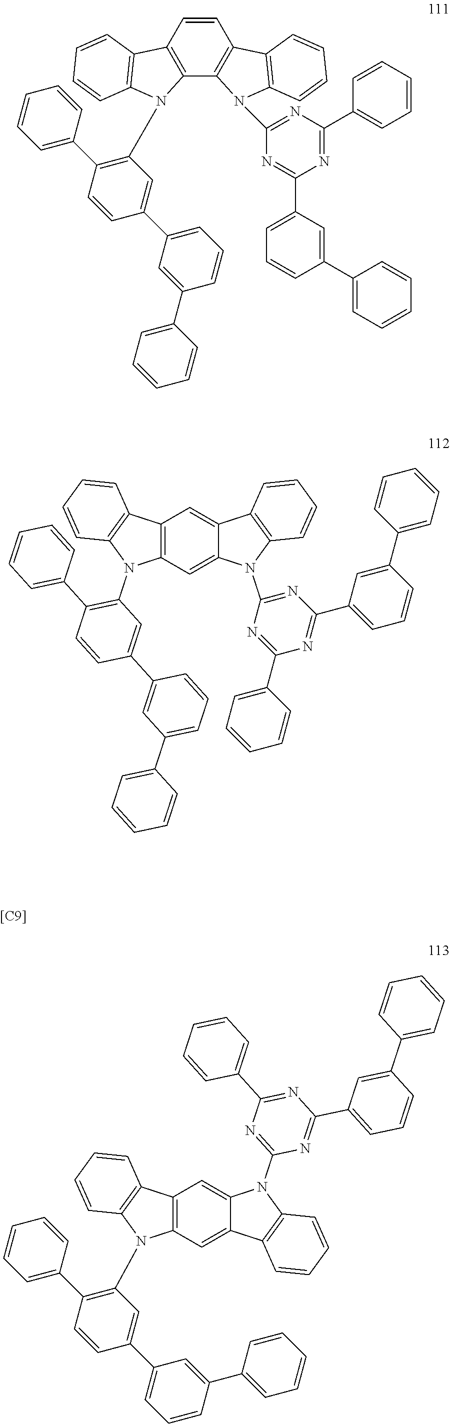

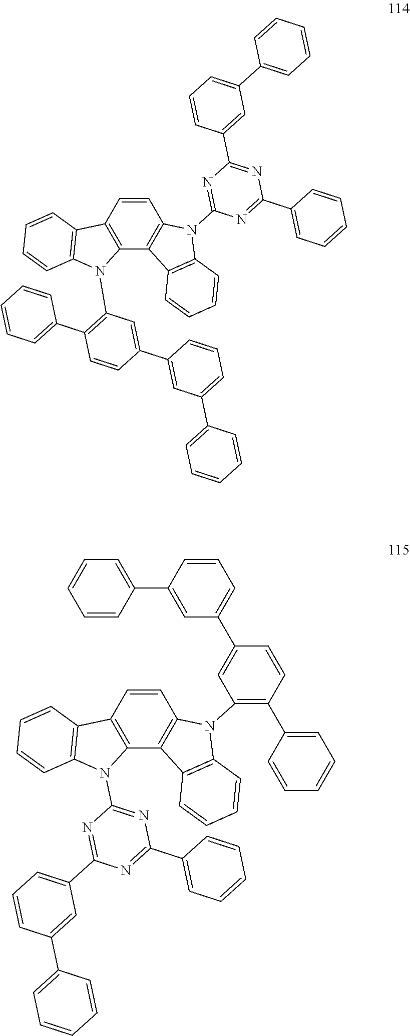

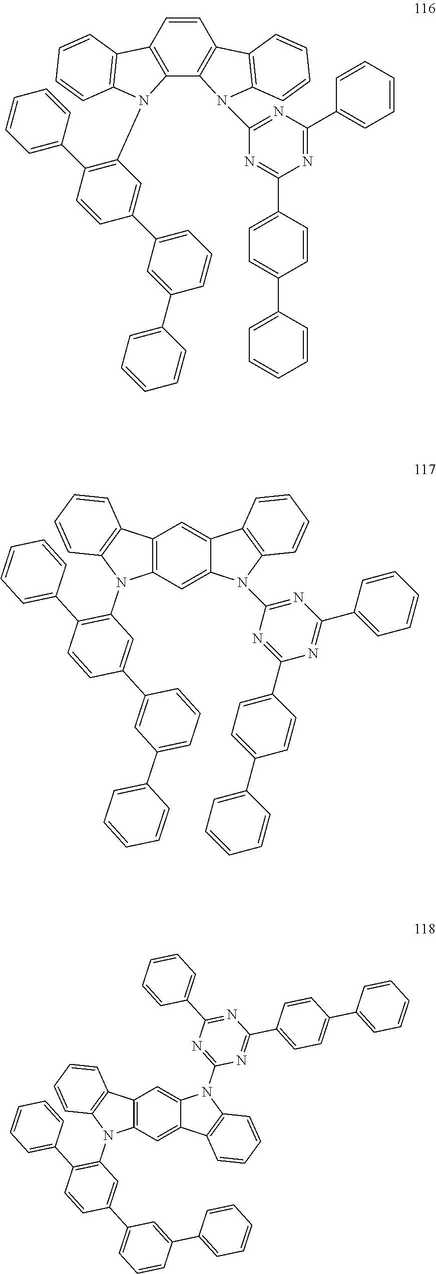

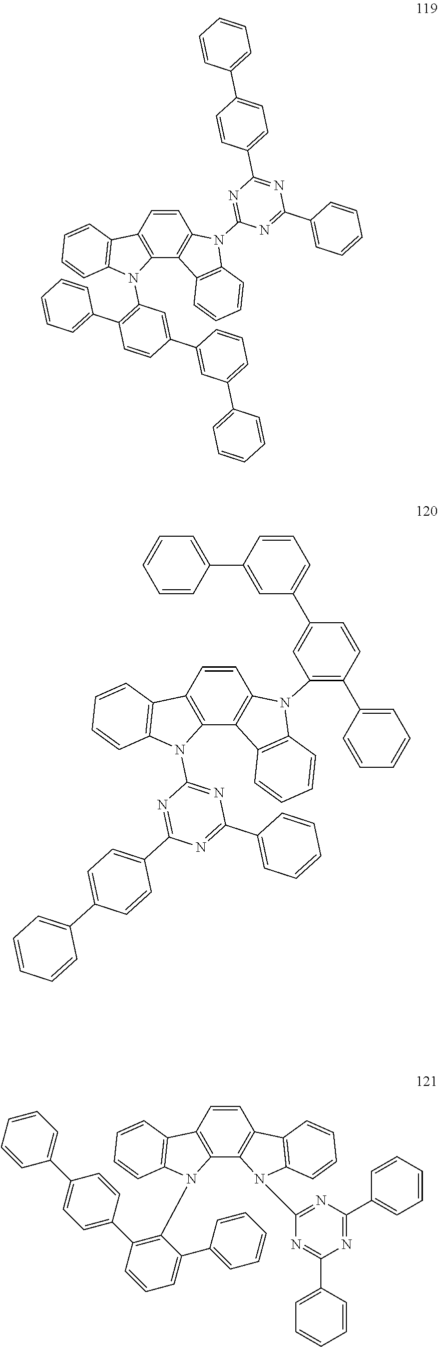

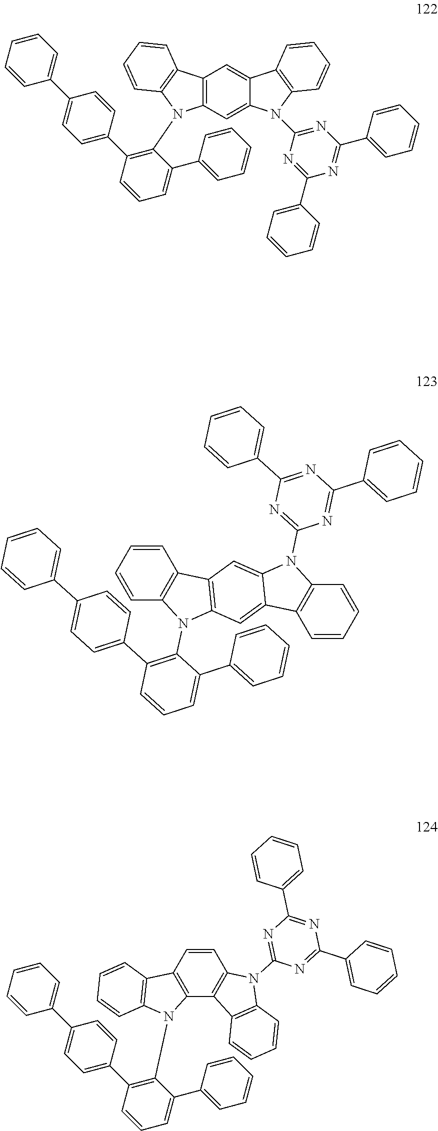

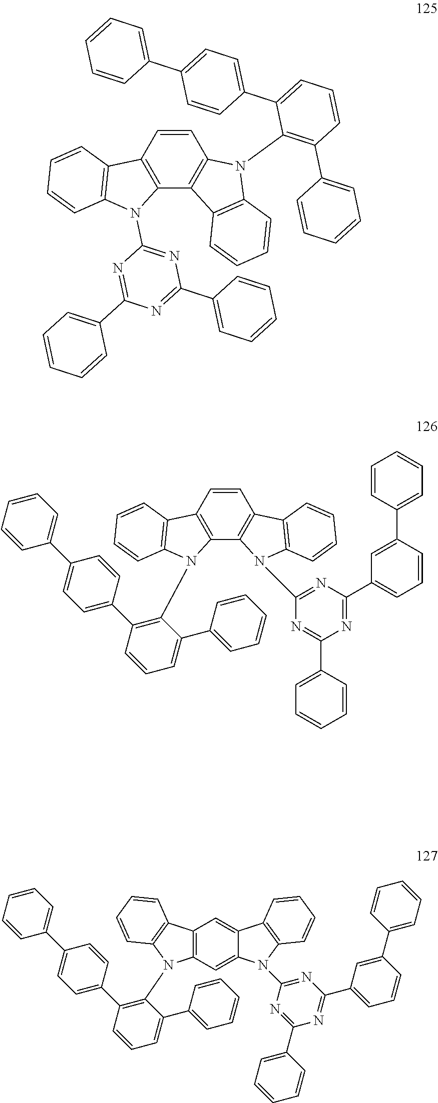

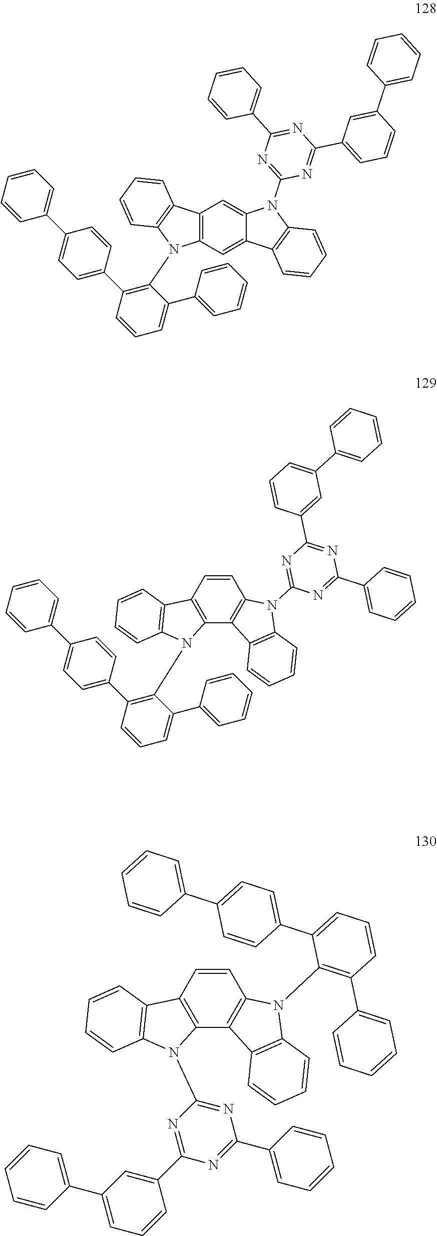

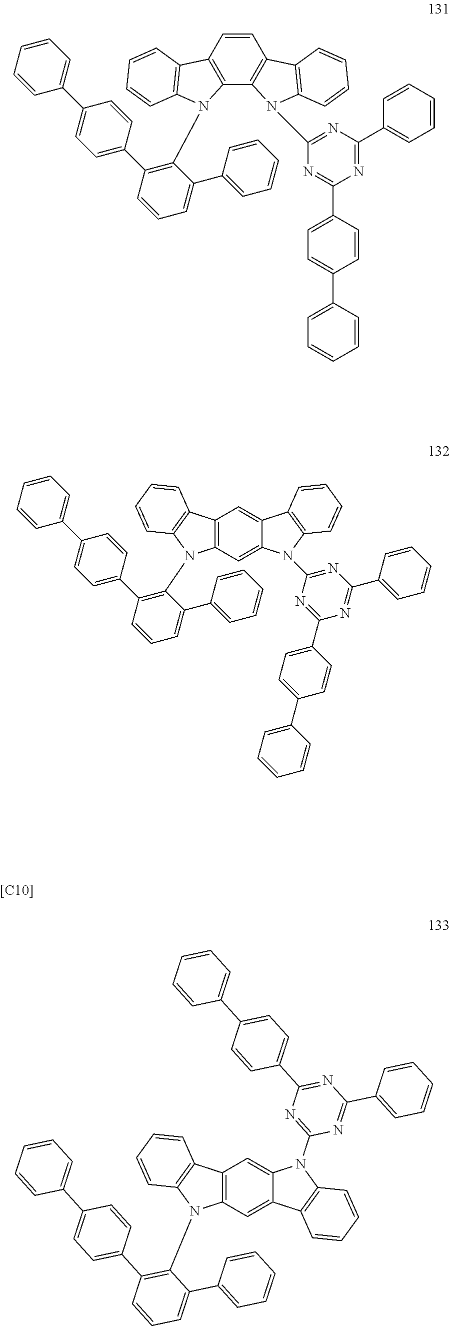

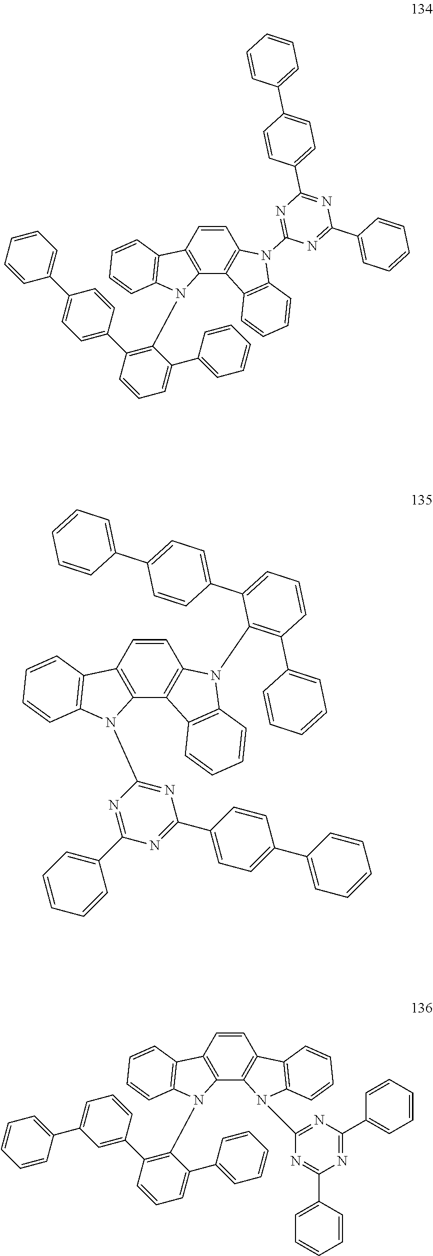

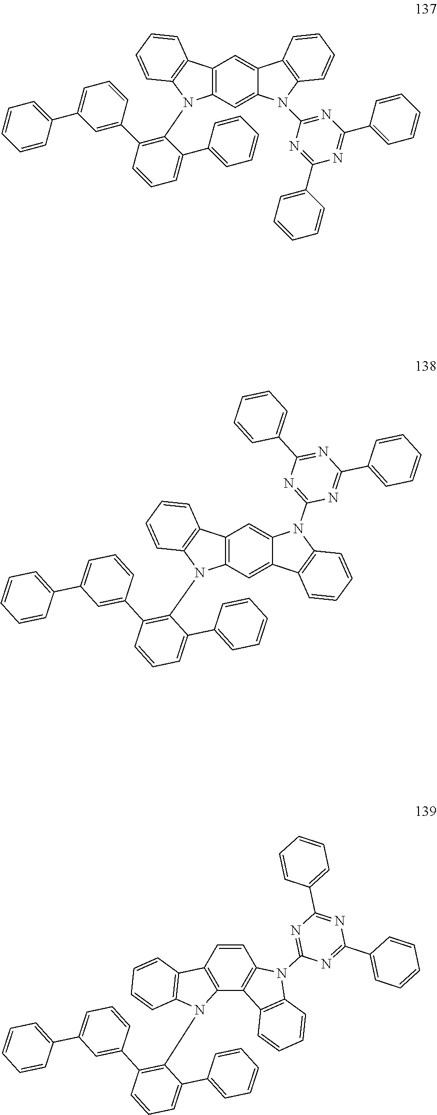

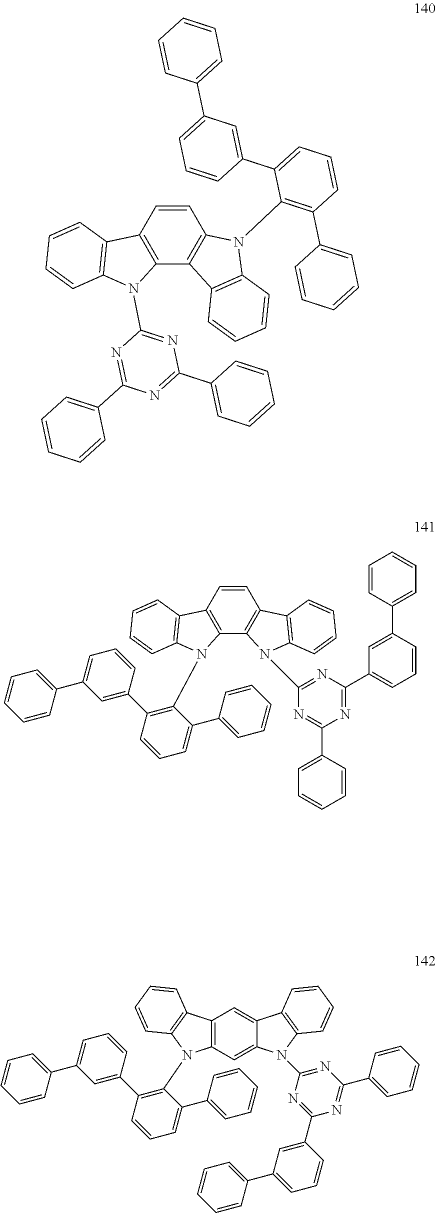

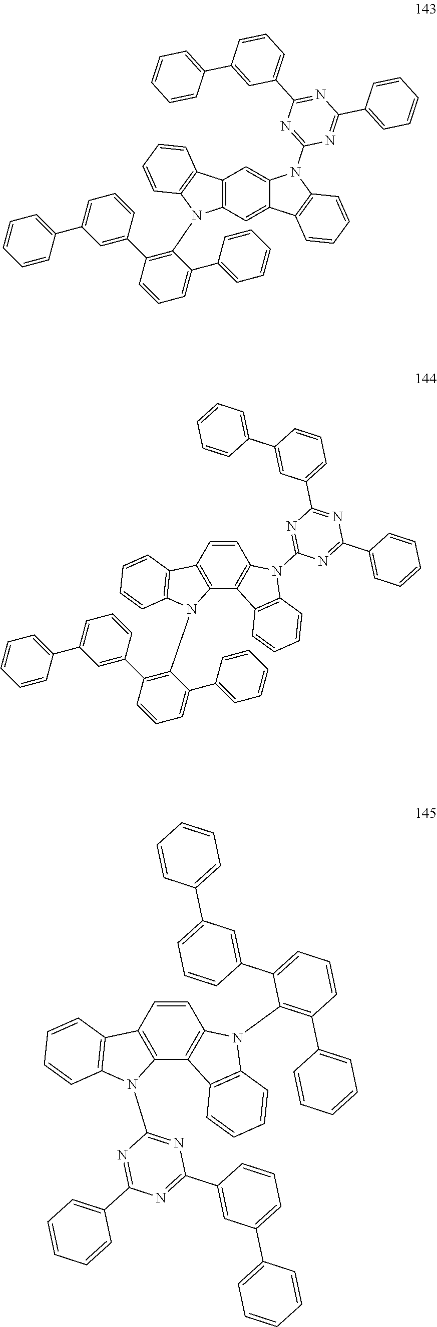

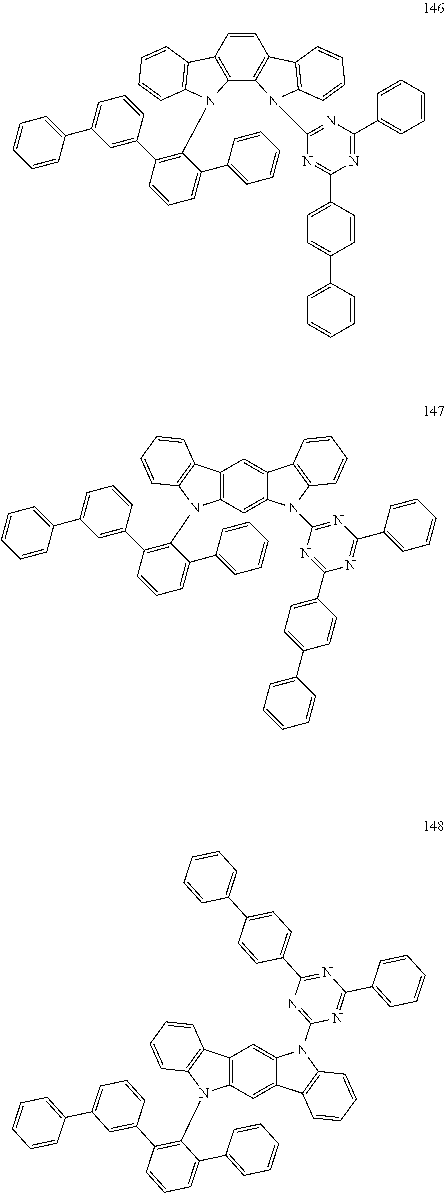

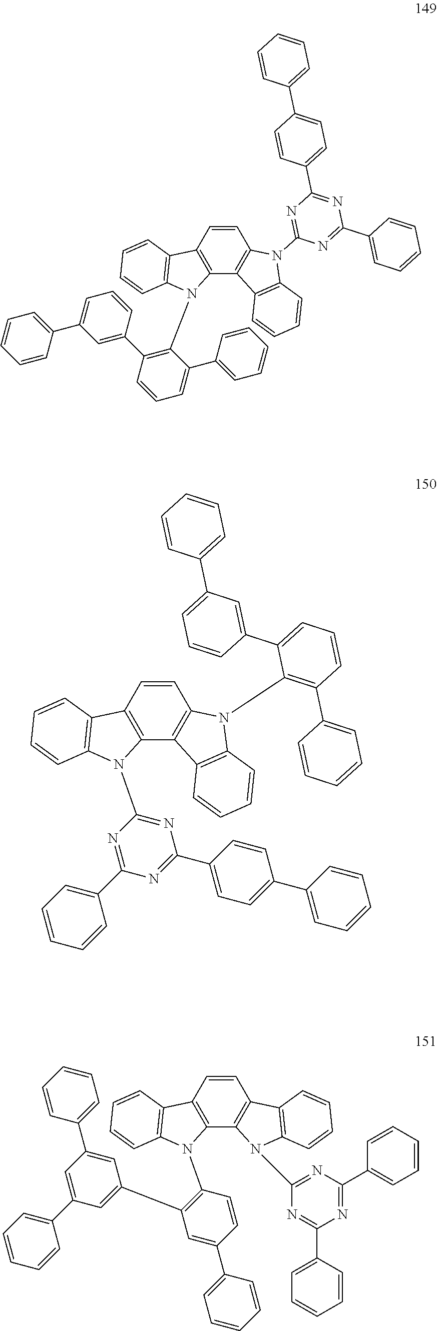

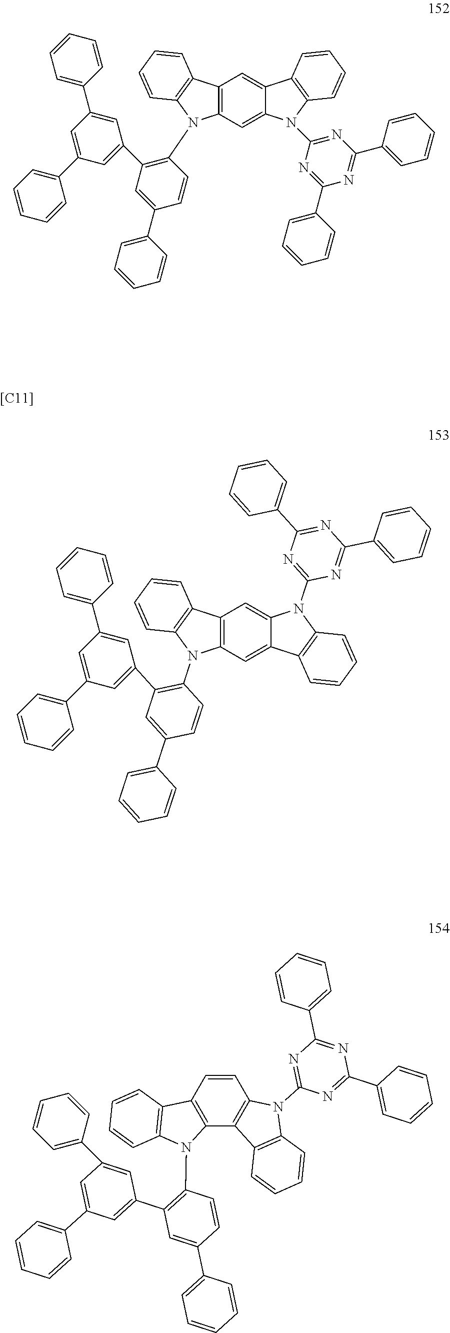

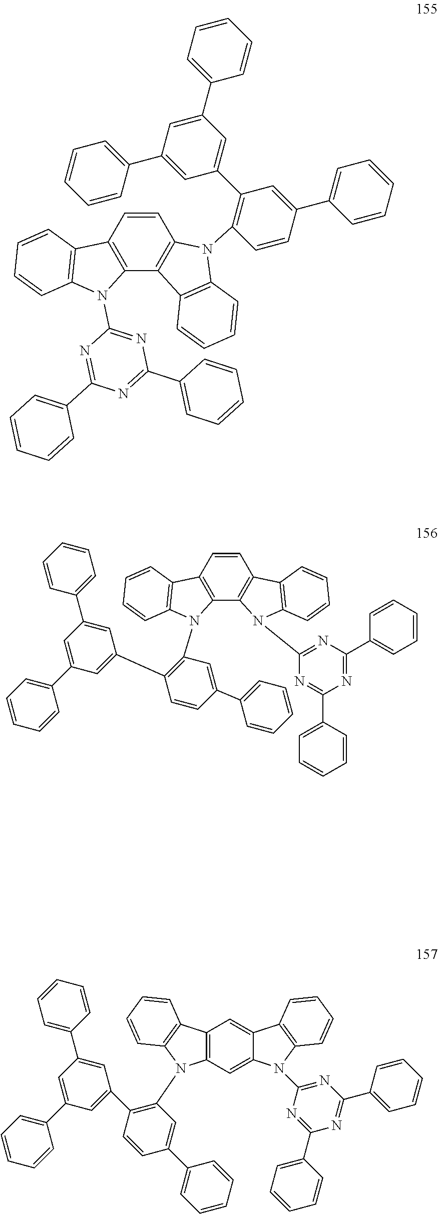

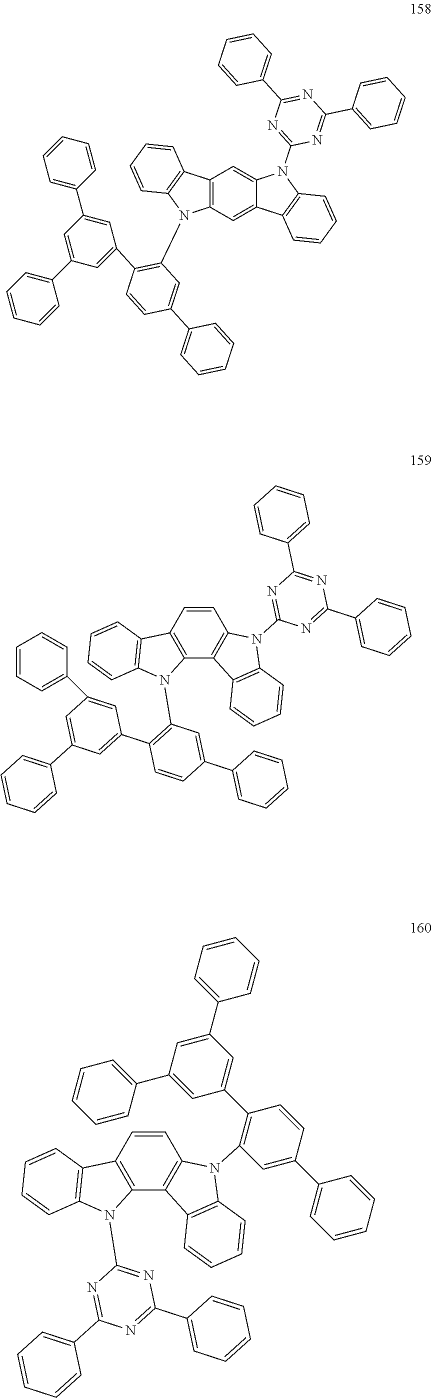

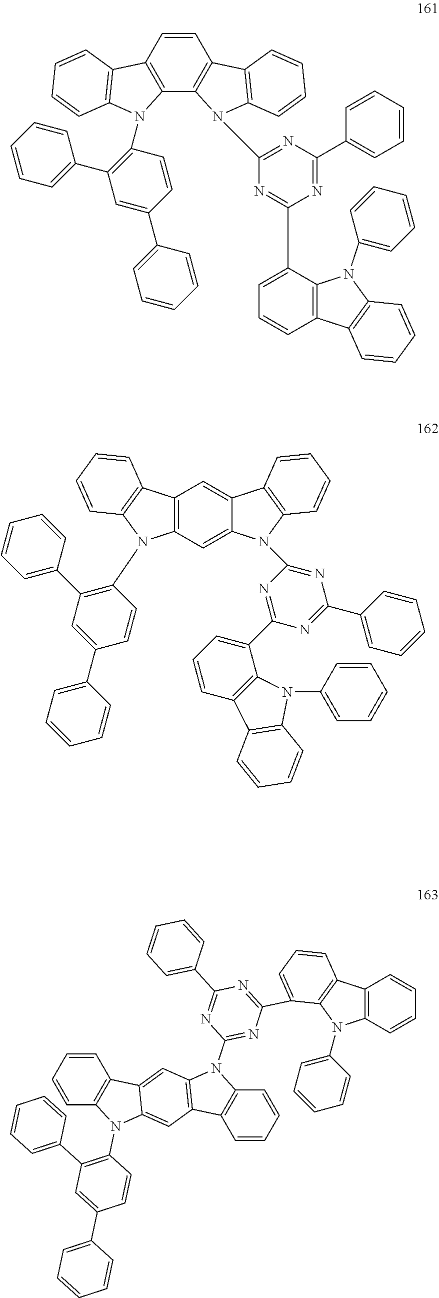

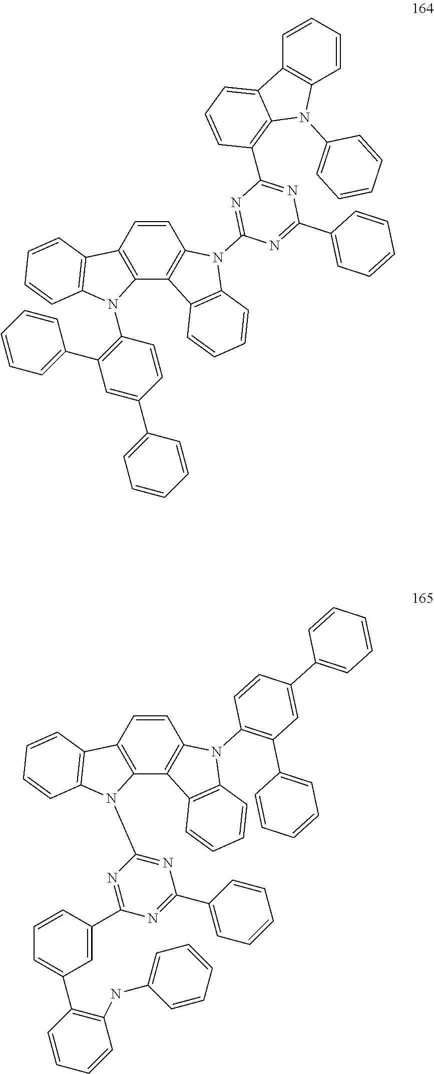

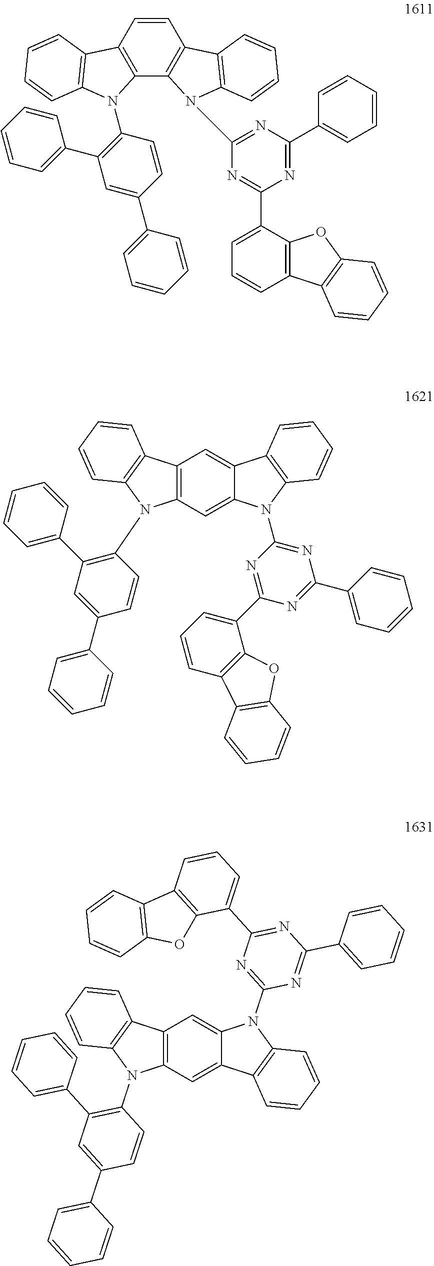

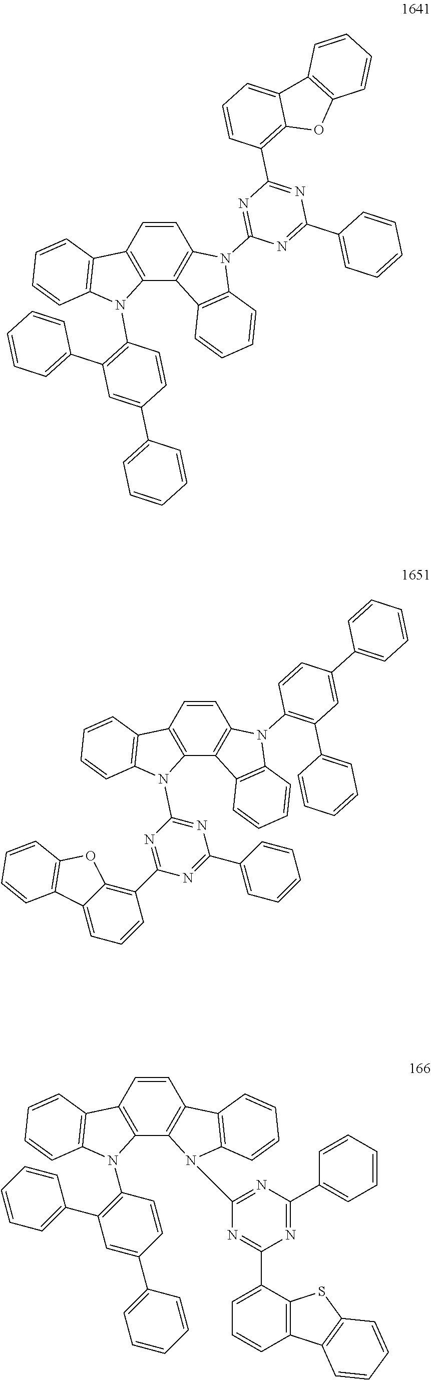

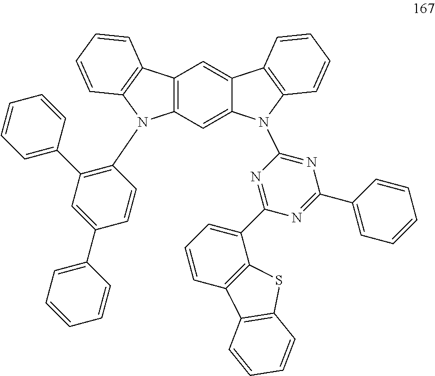

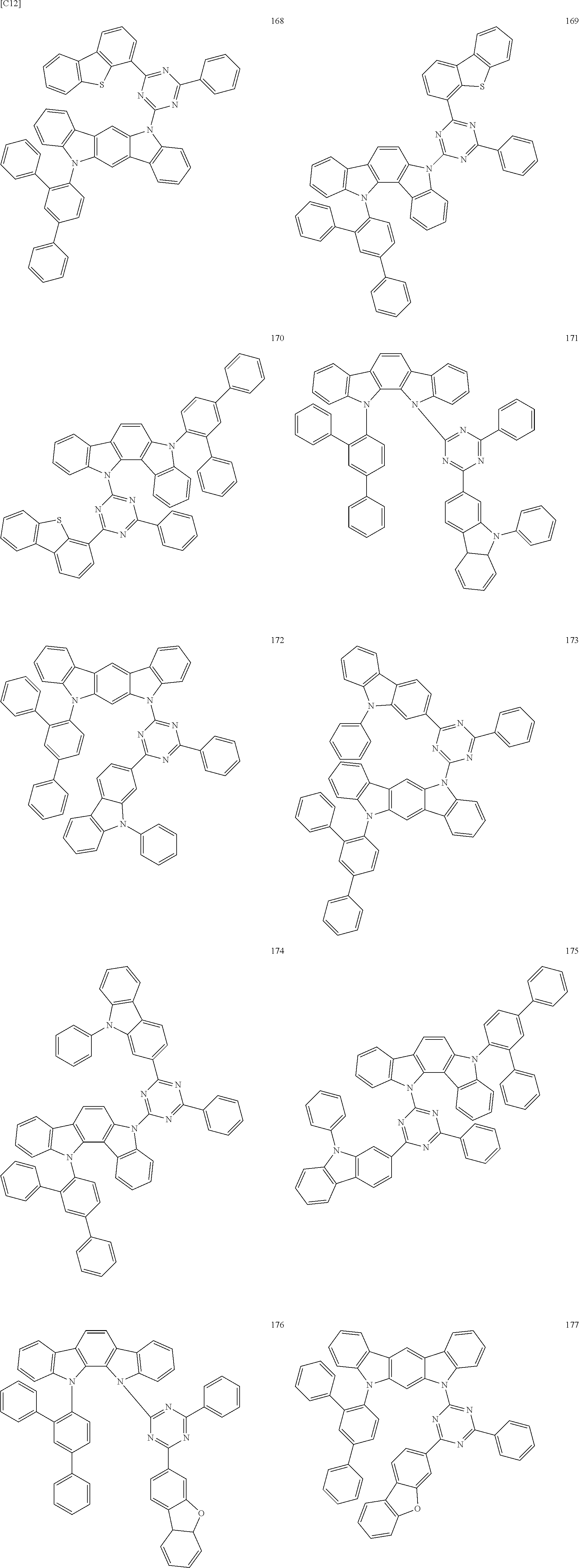

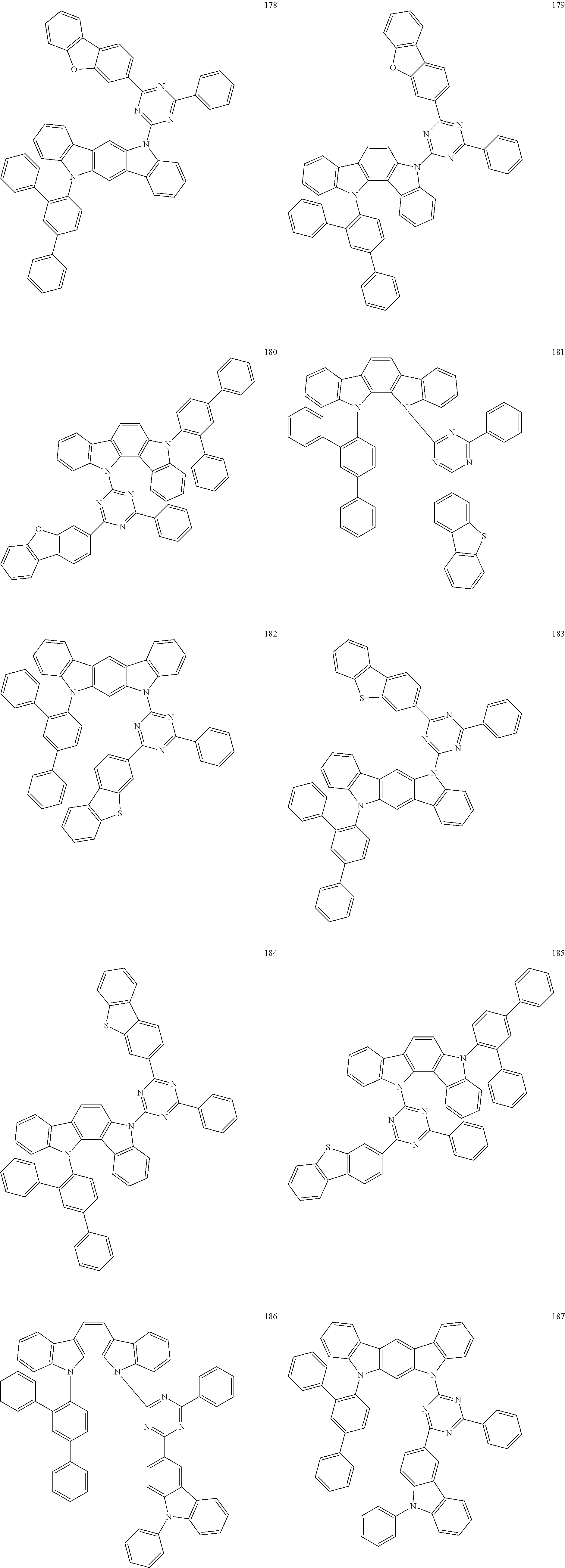

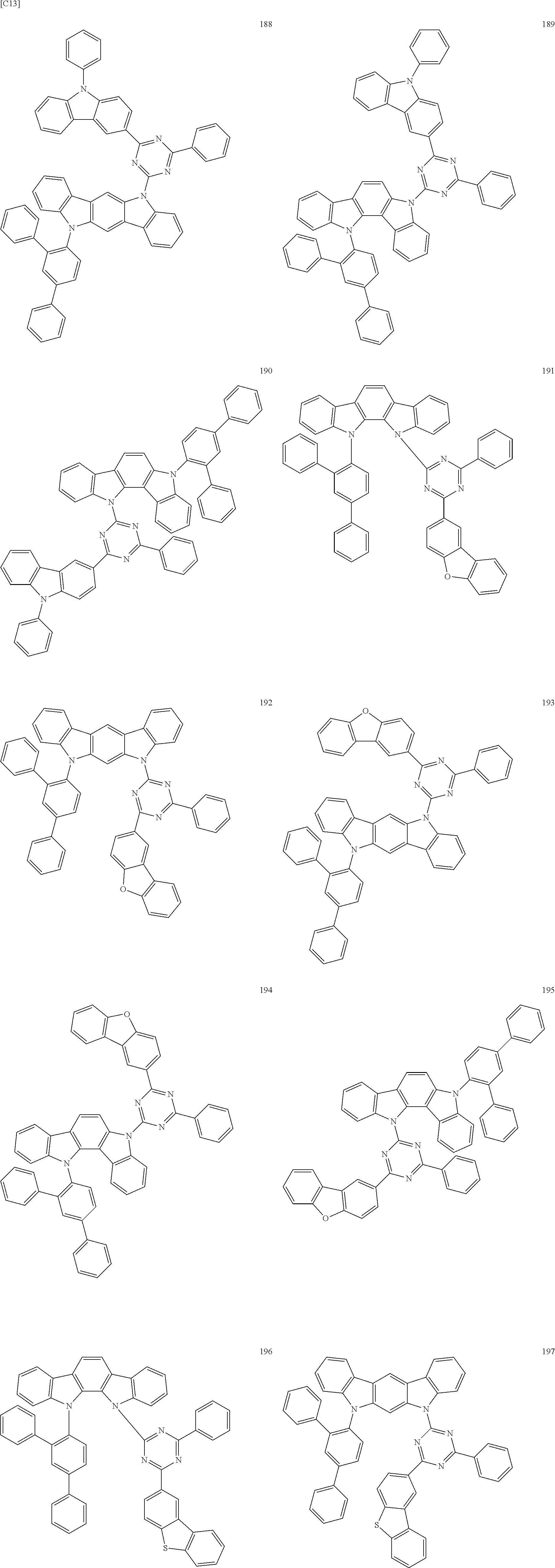

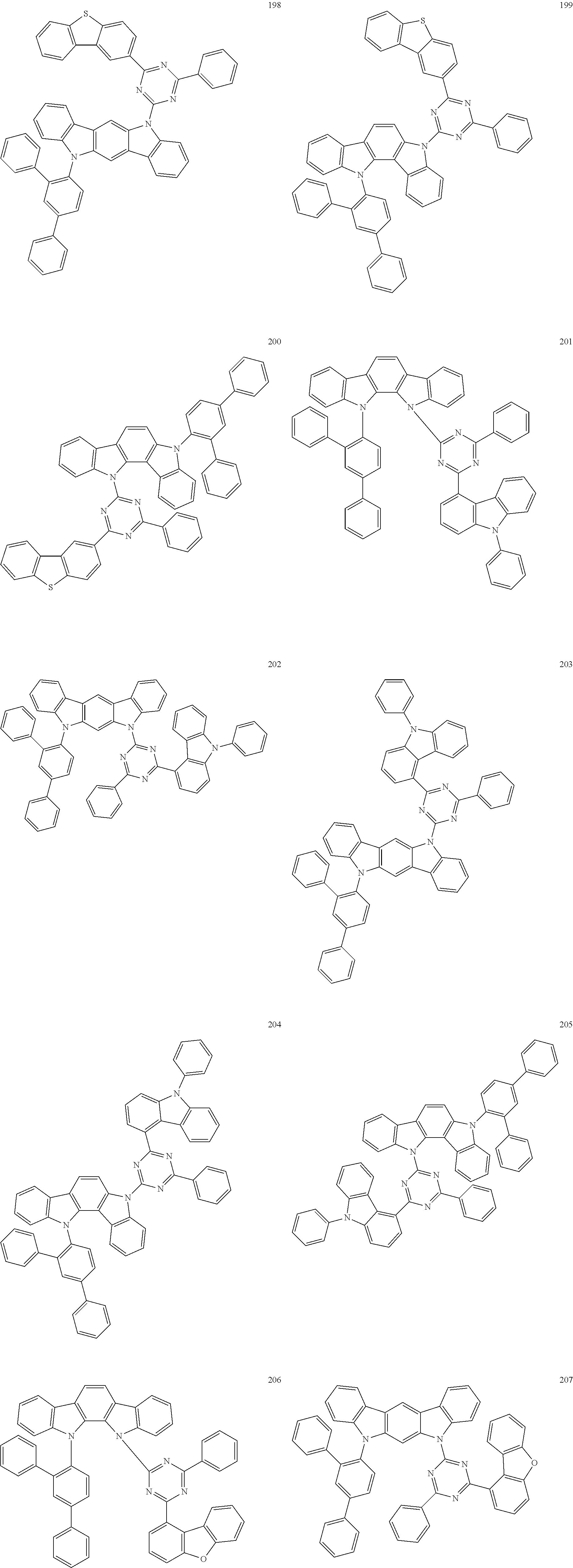

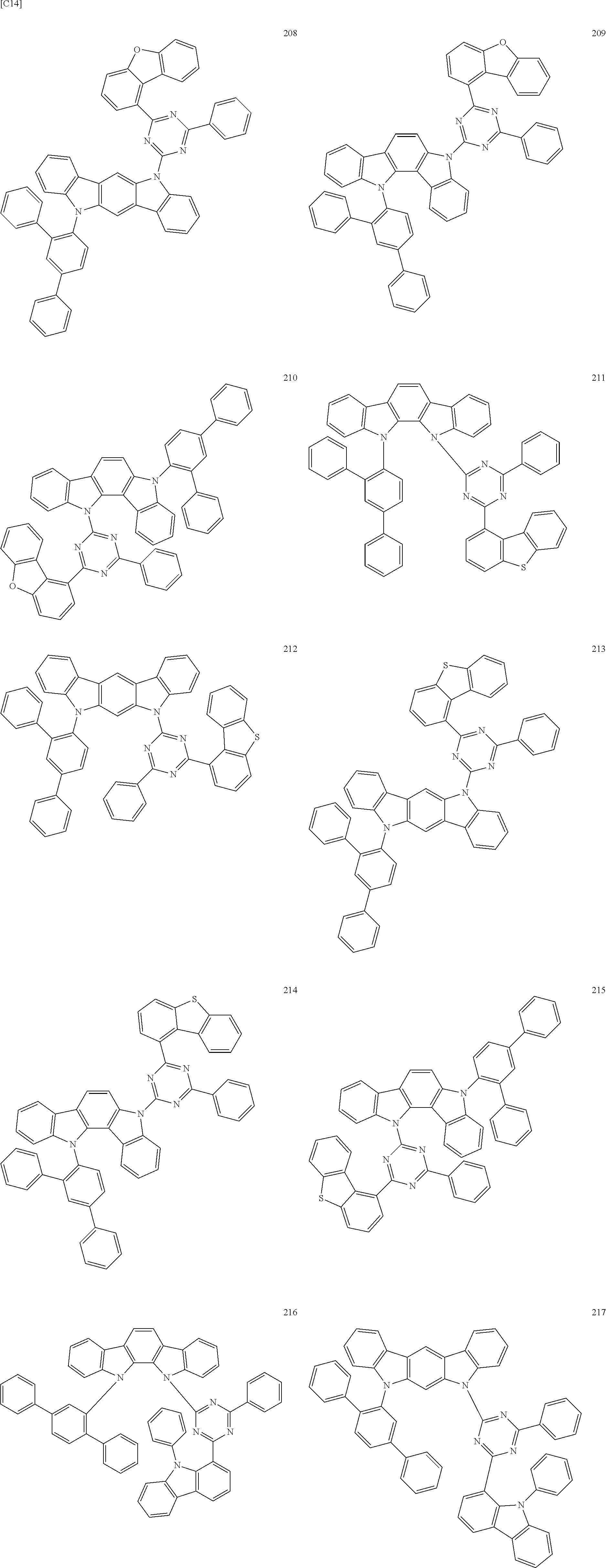

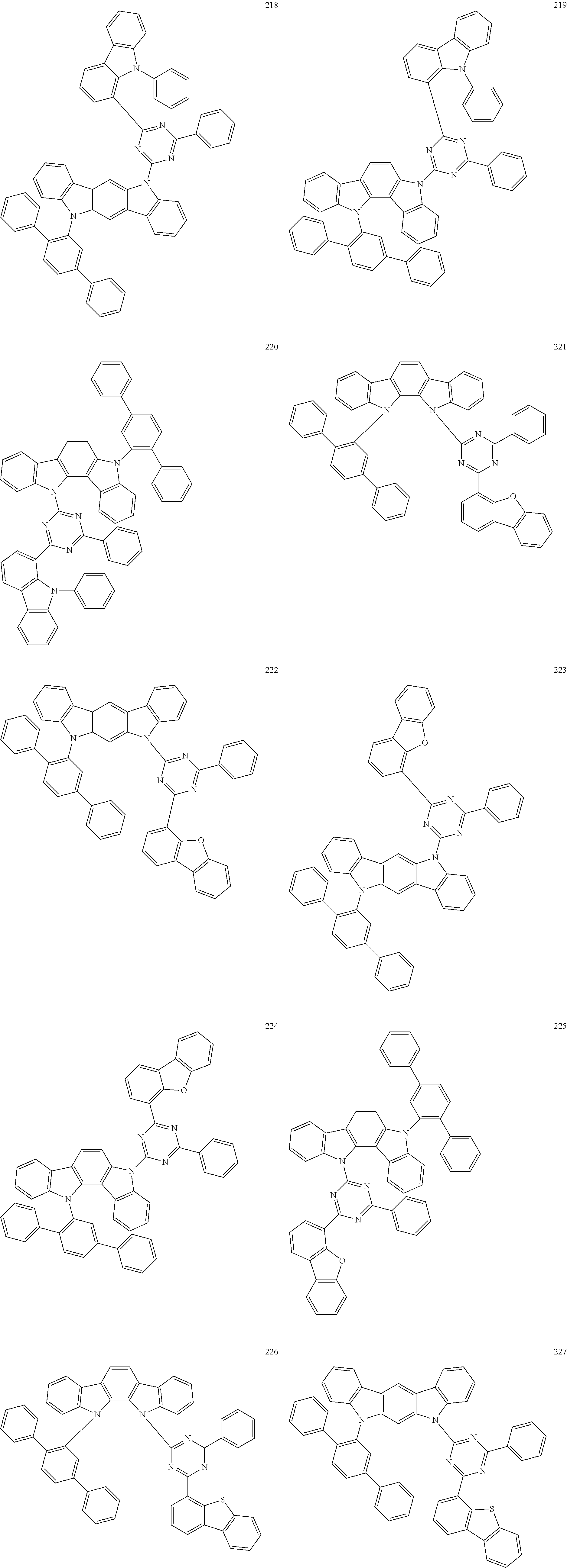

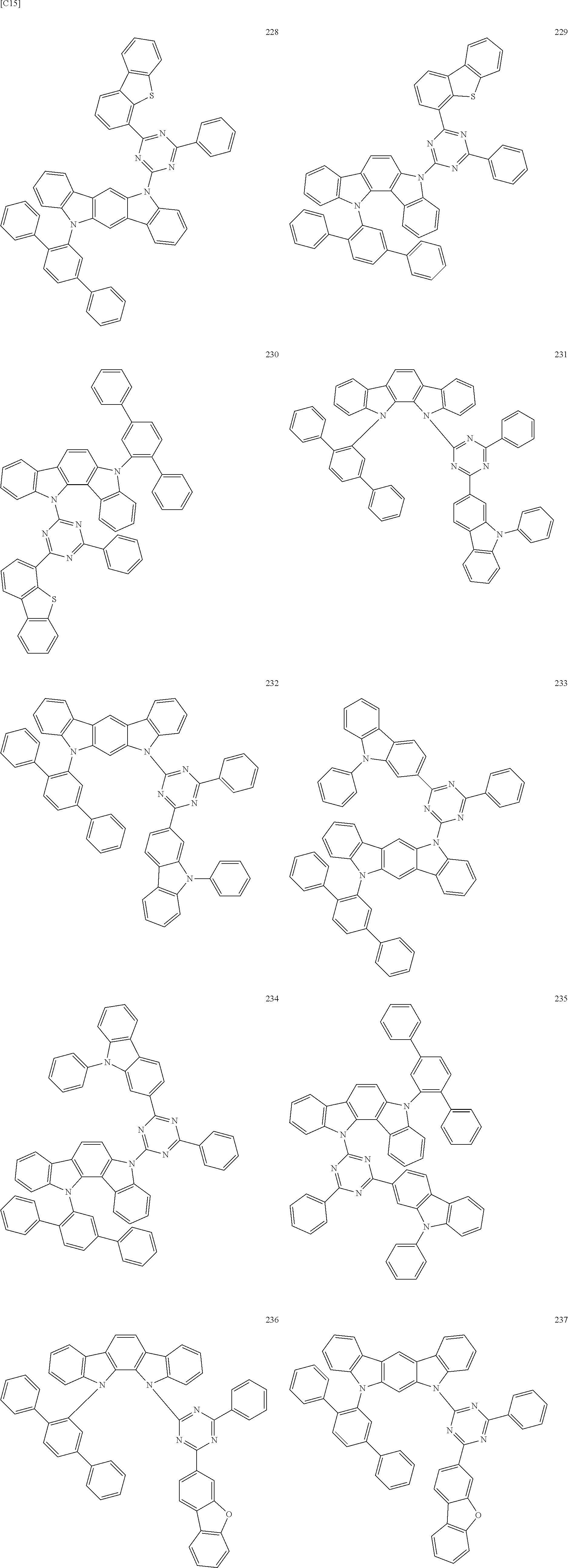

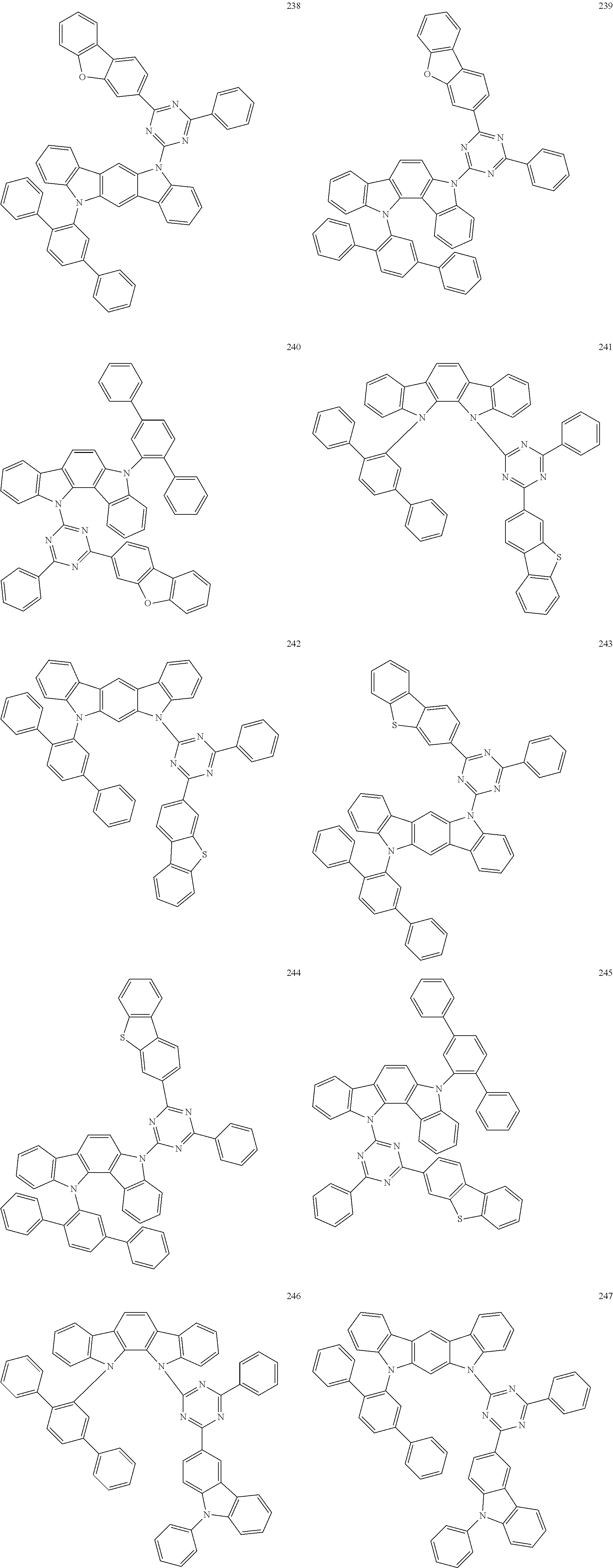

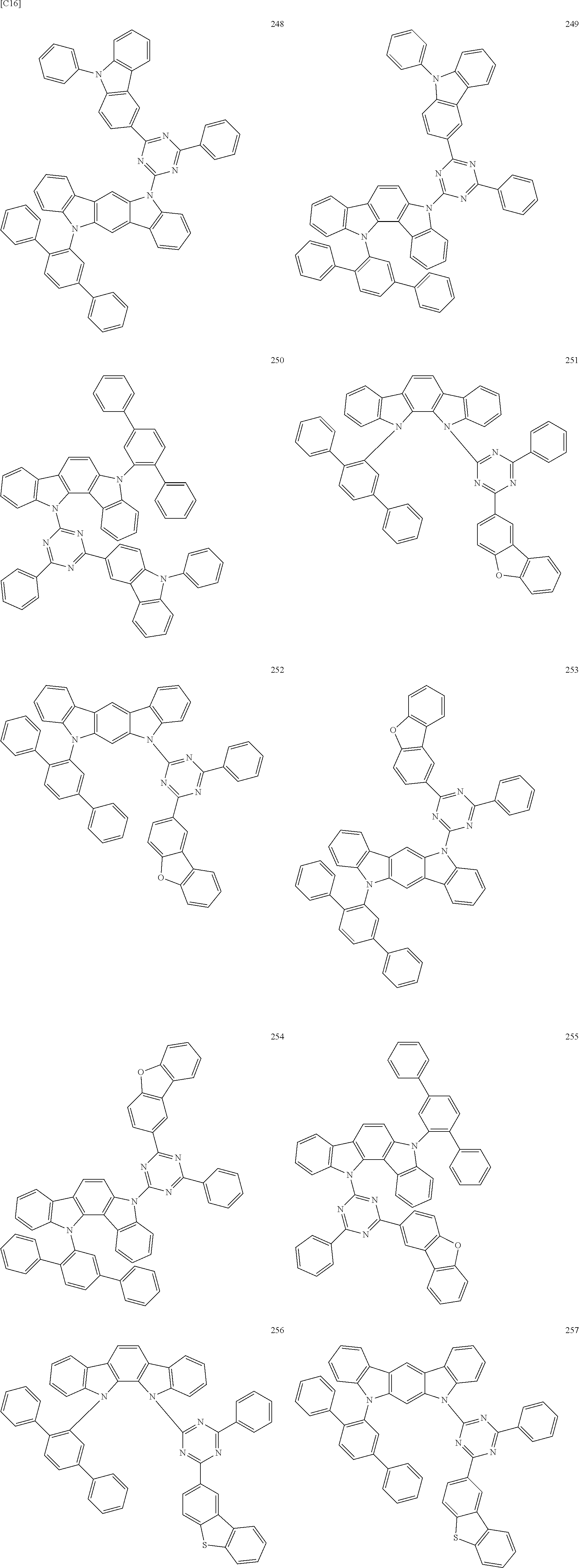

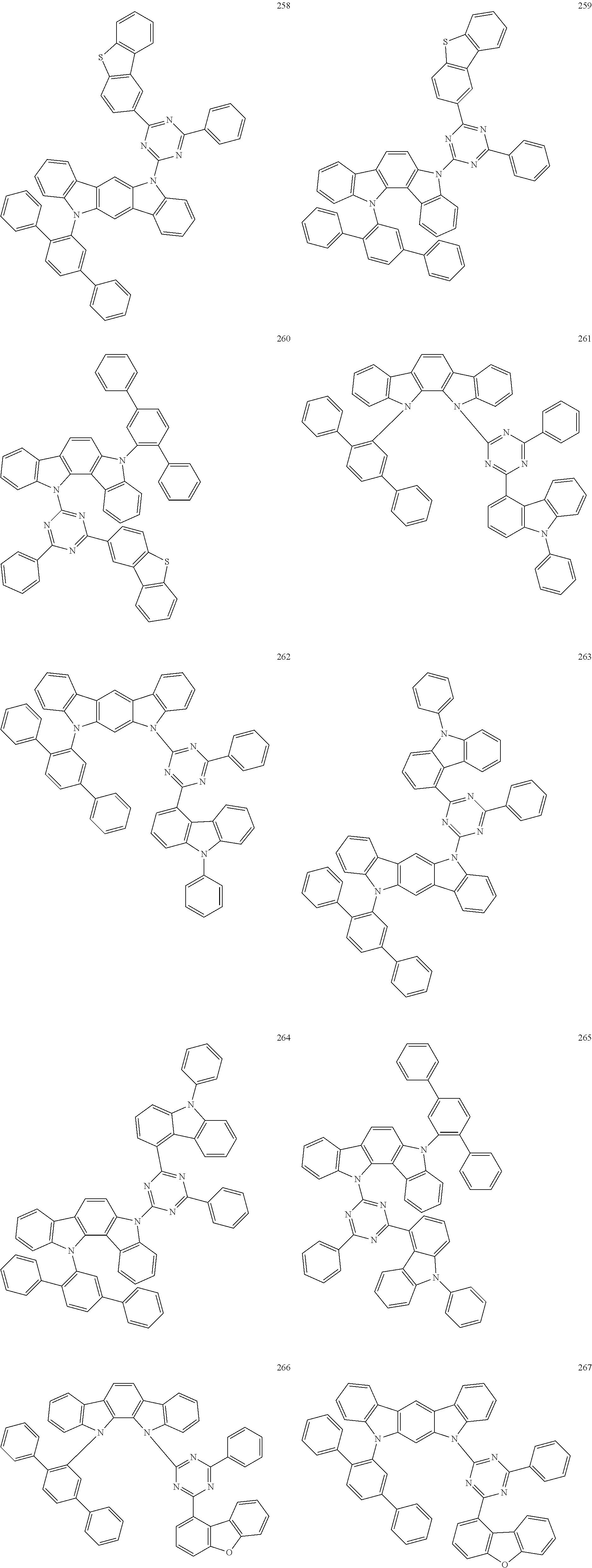

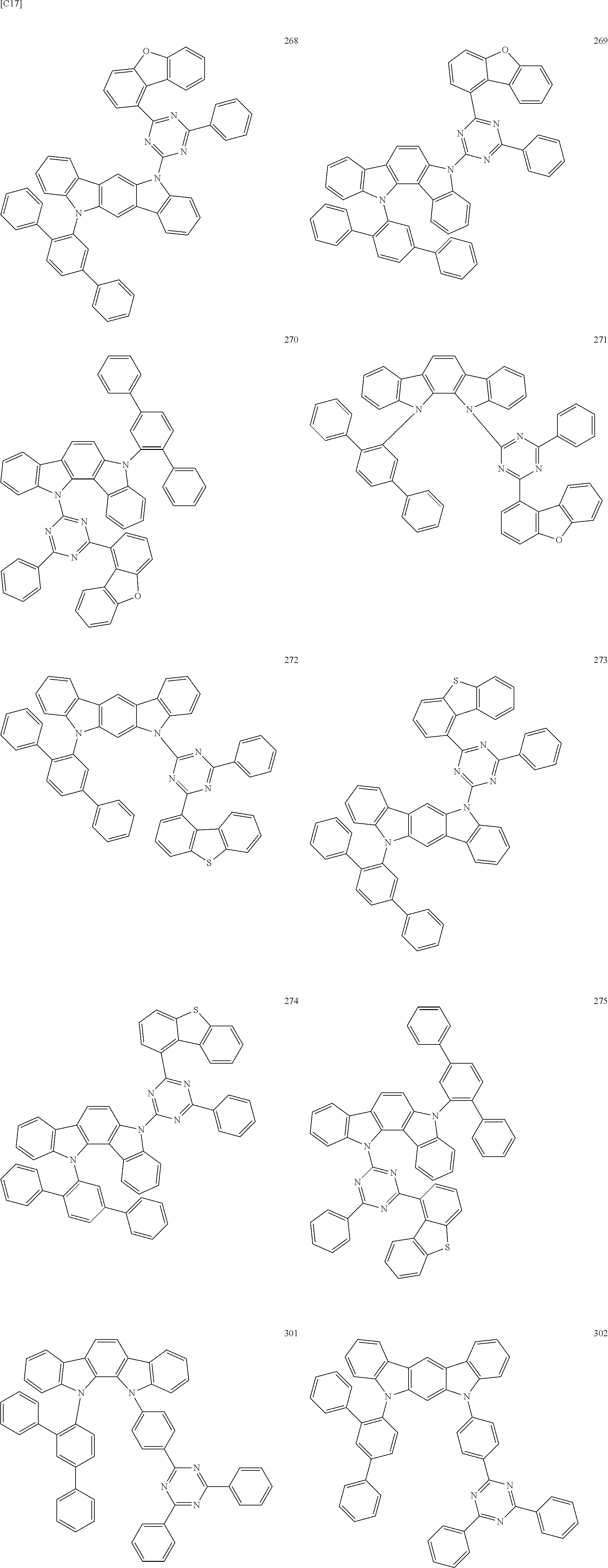

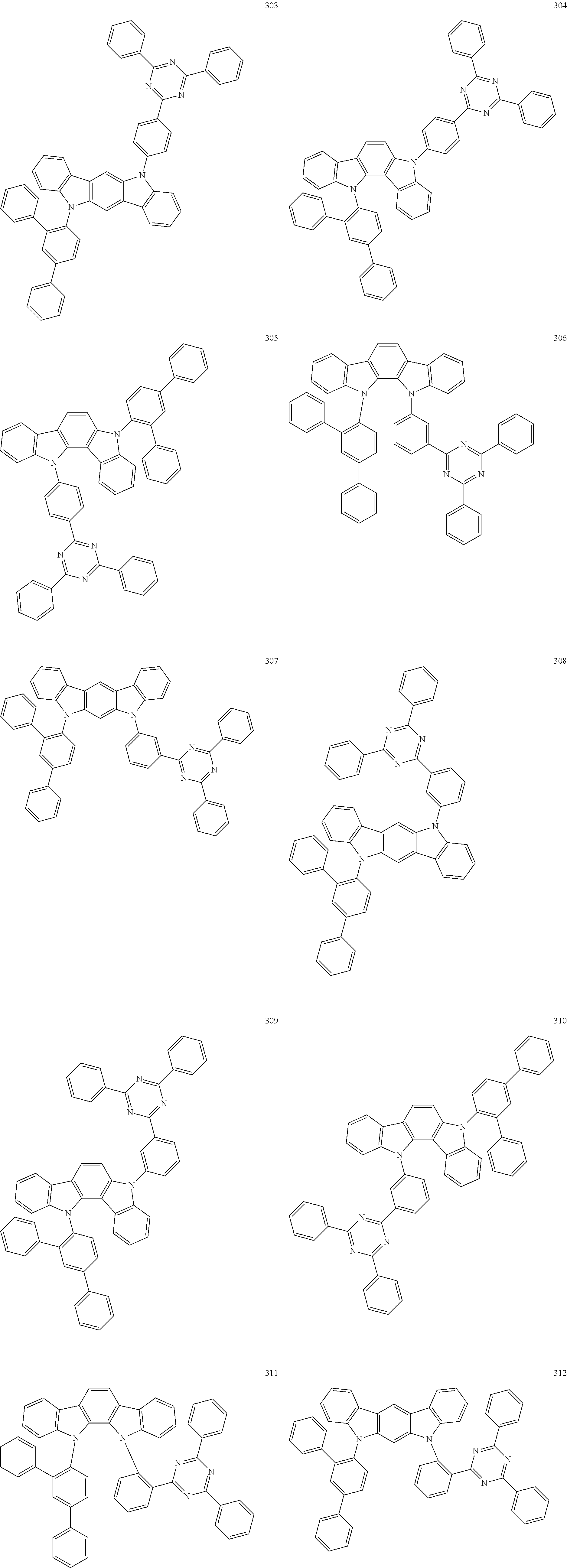

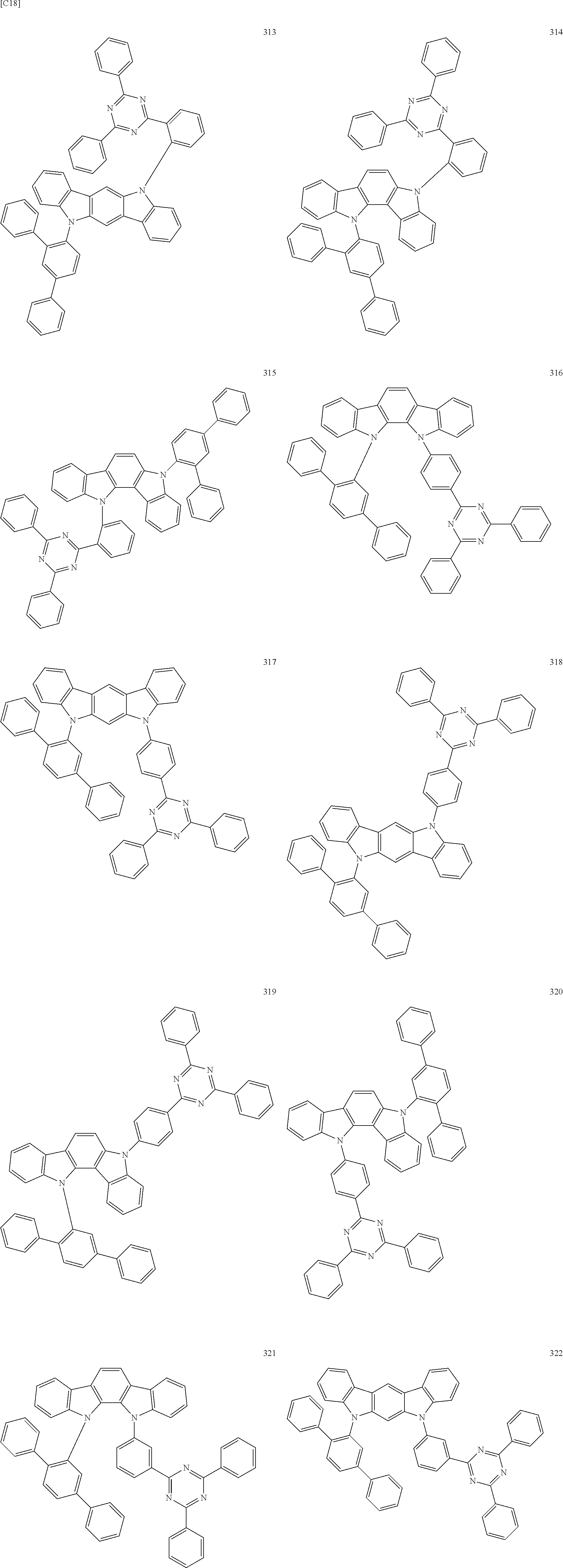

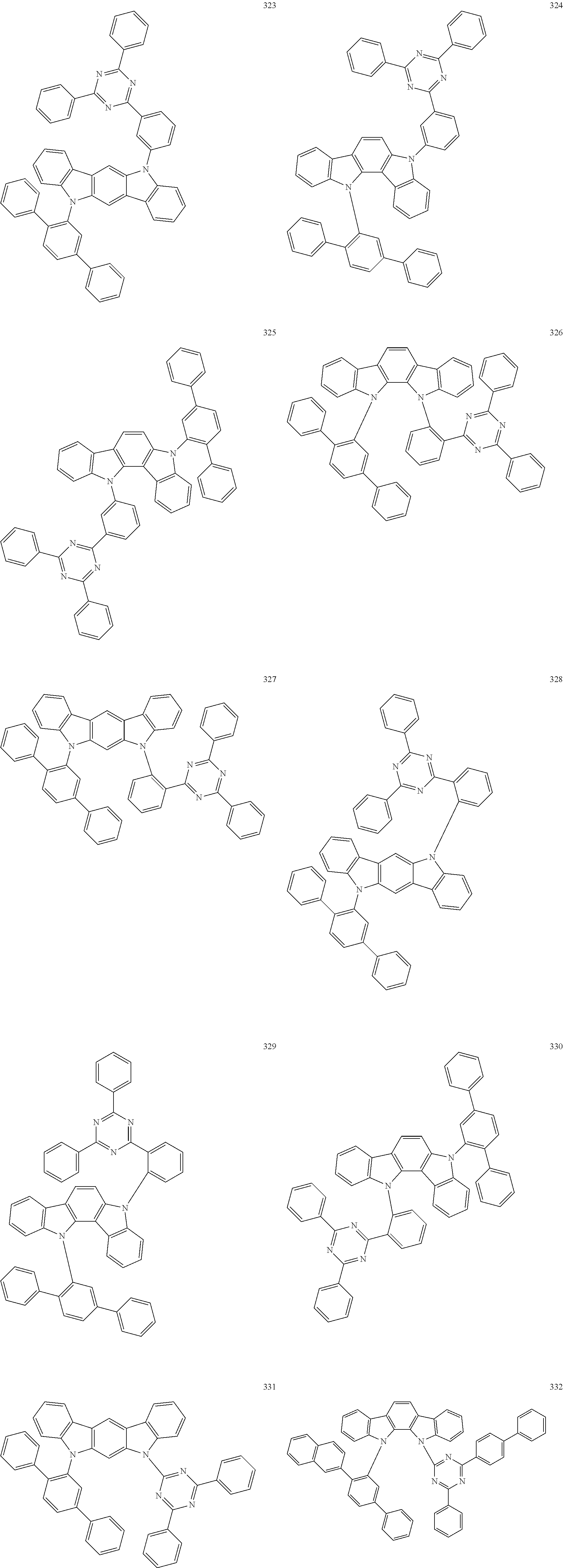

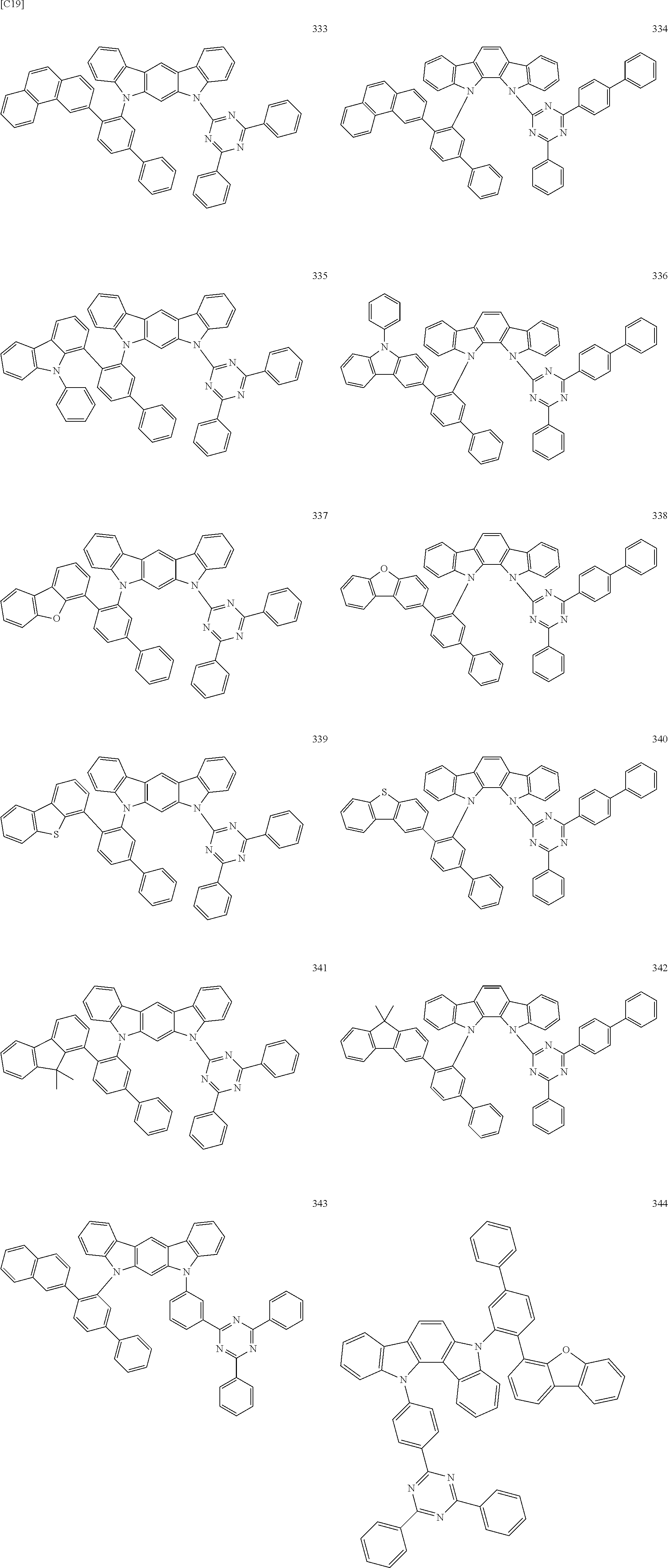

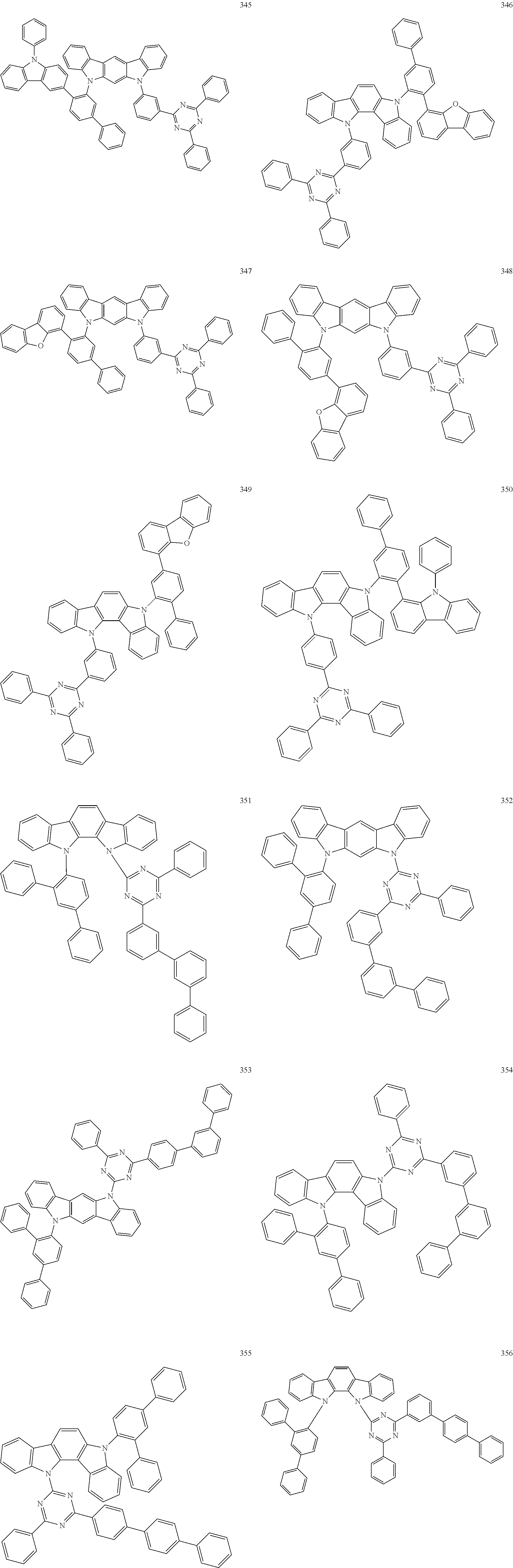

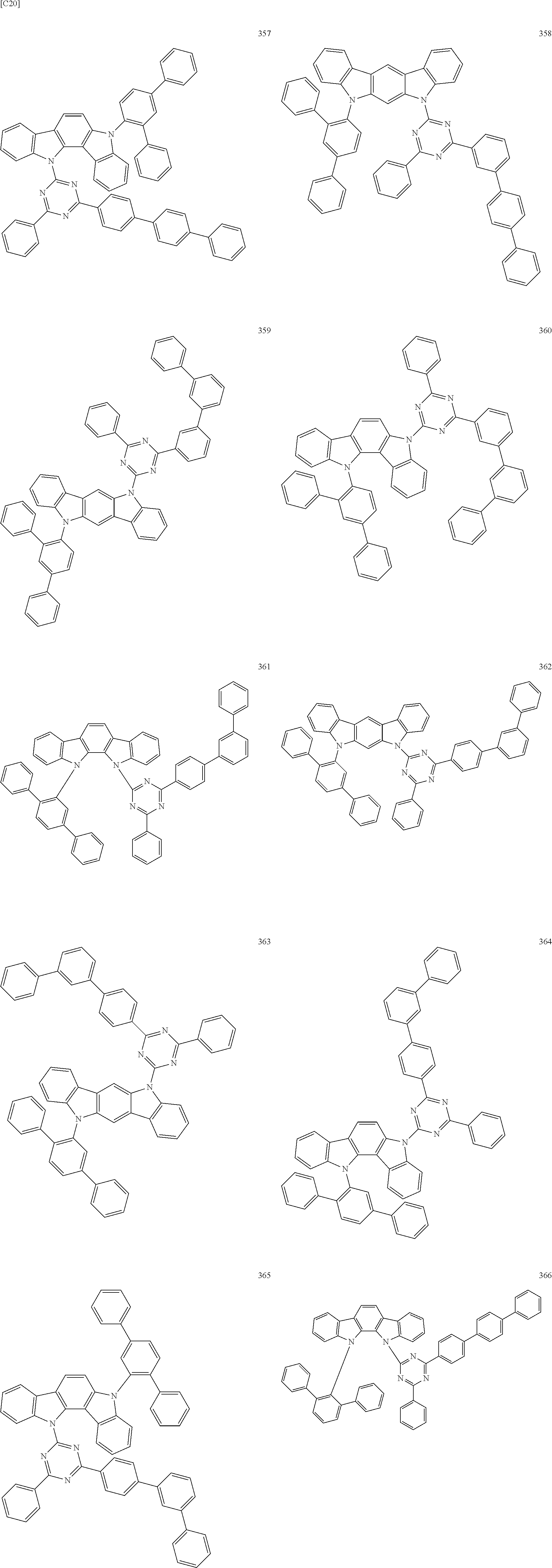

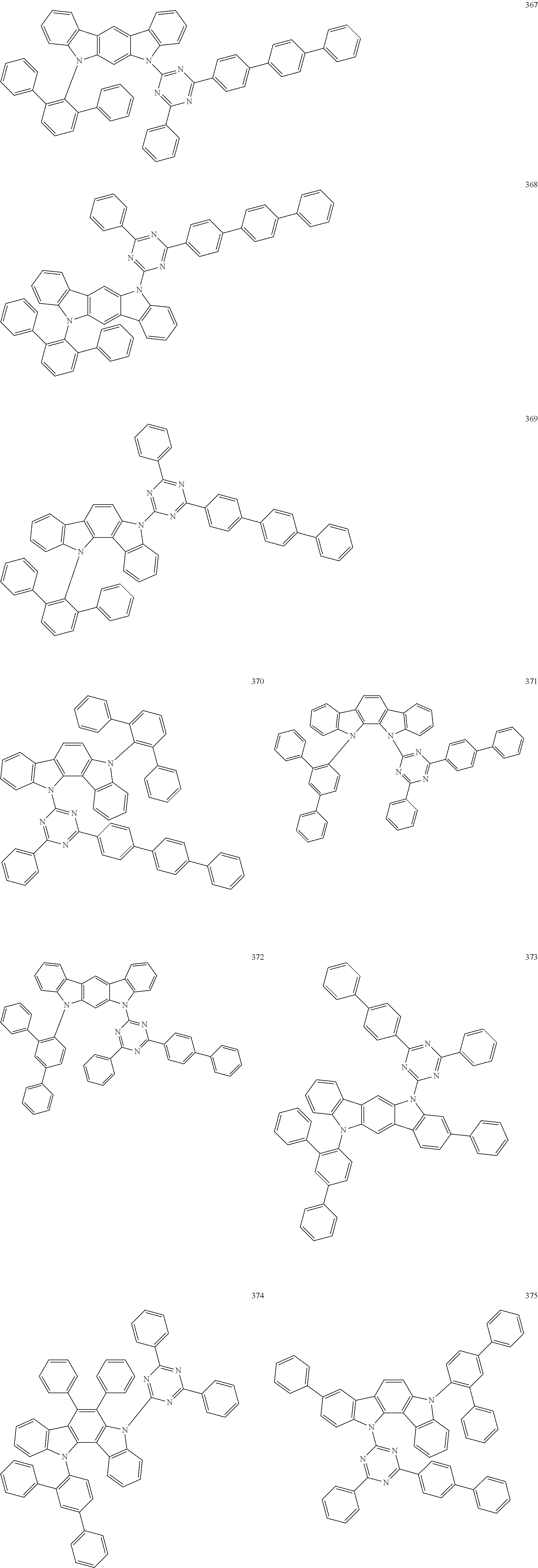

Specific examples of the compounds represented by the general formula (10) are shown below, but are not limited to these exemplified compounds. Herein, m indicates the number of substitutions of deuterium on average.

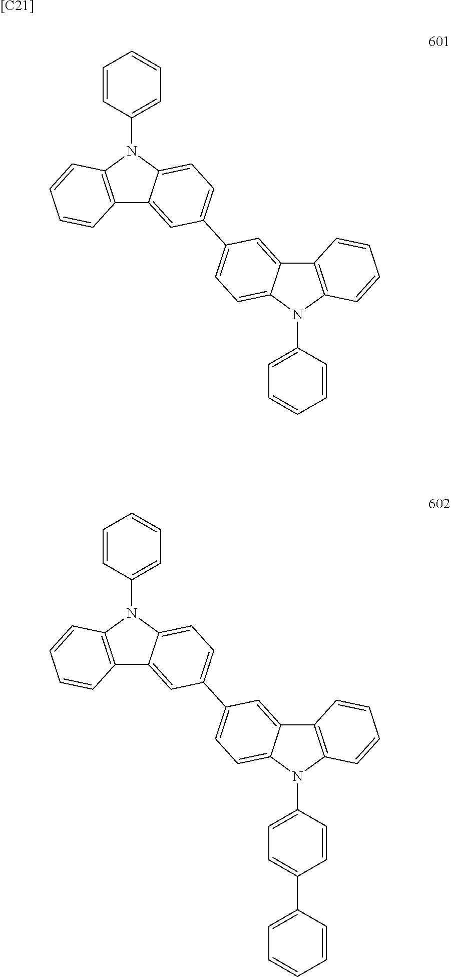

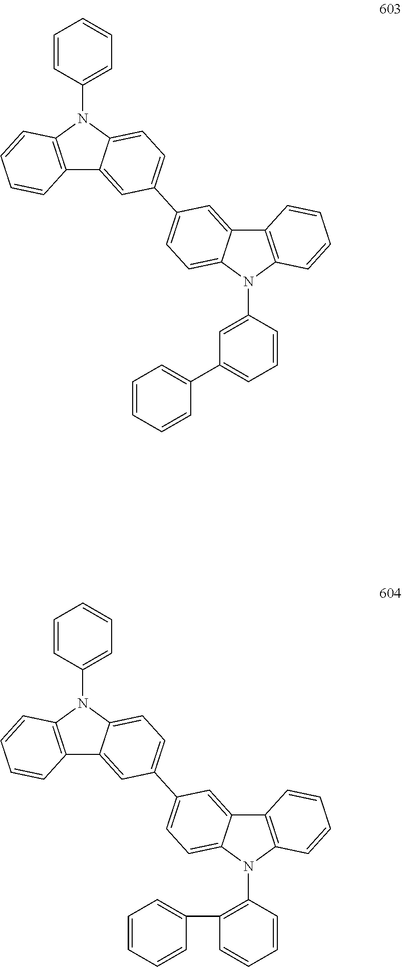

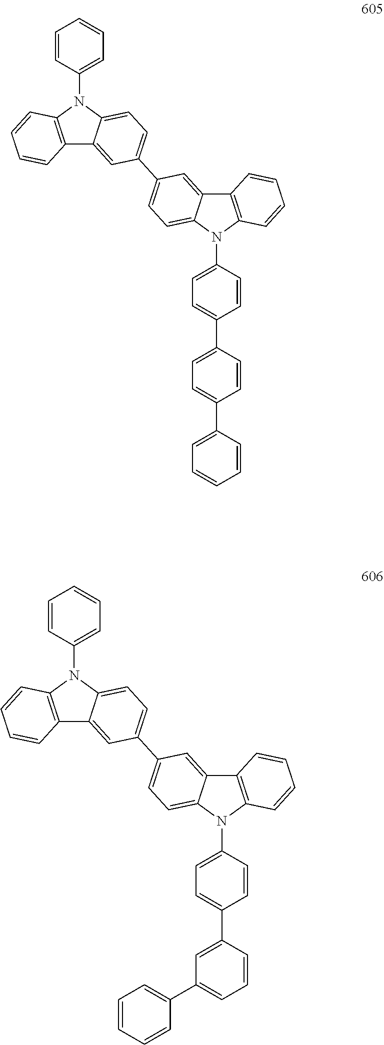

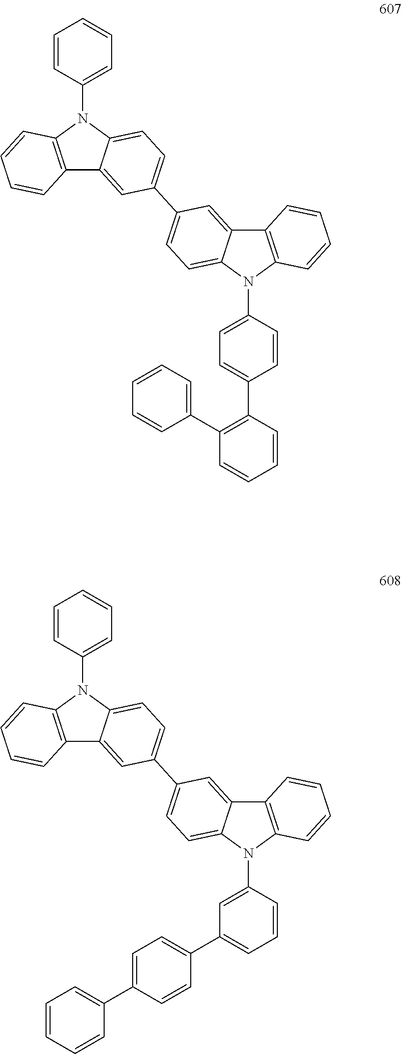

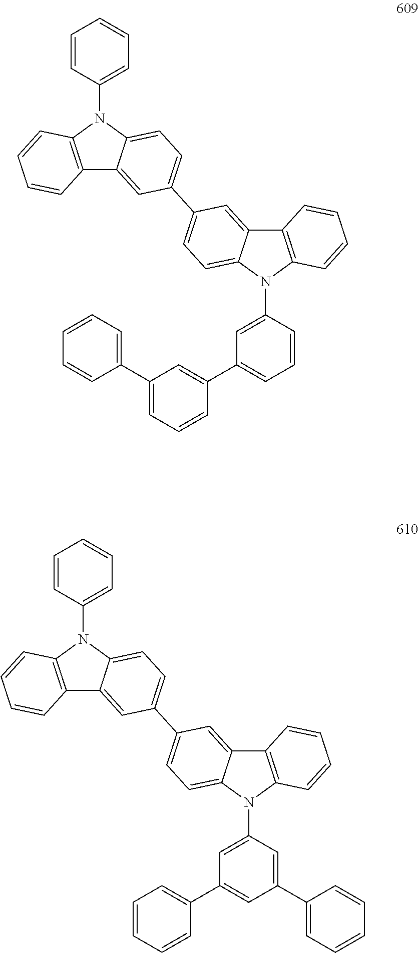

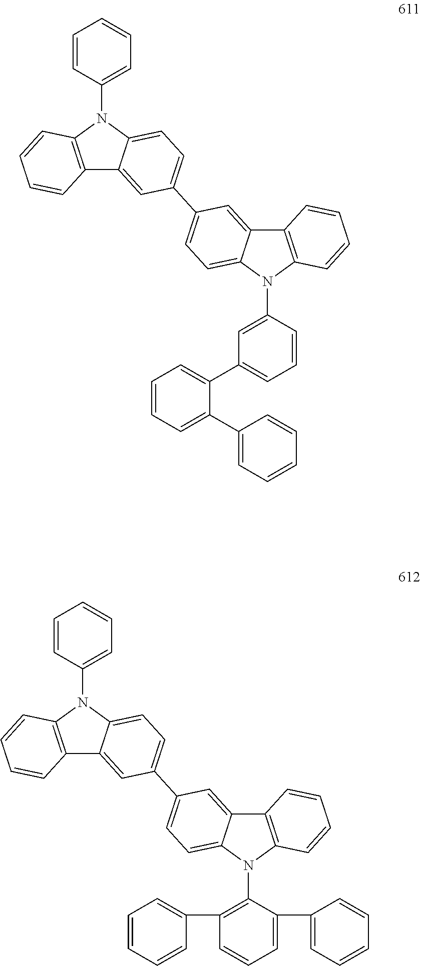

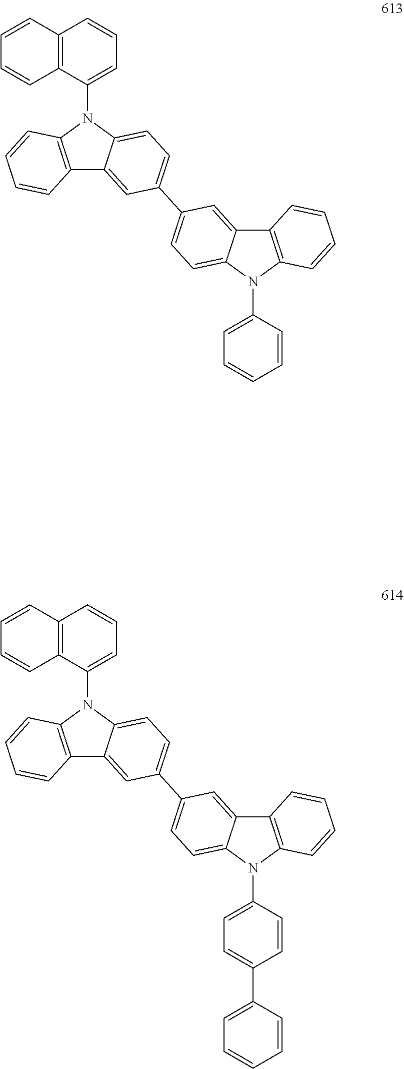

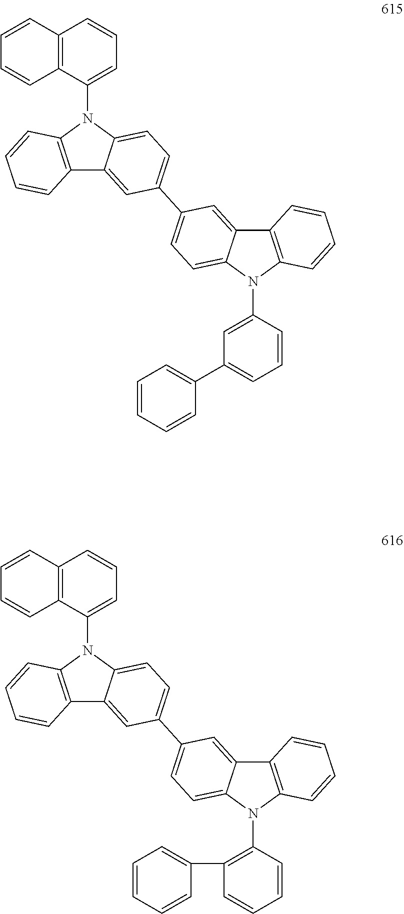

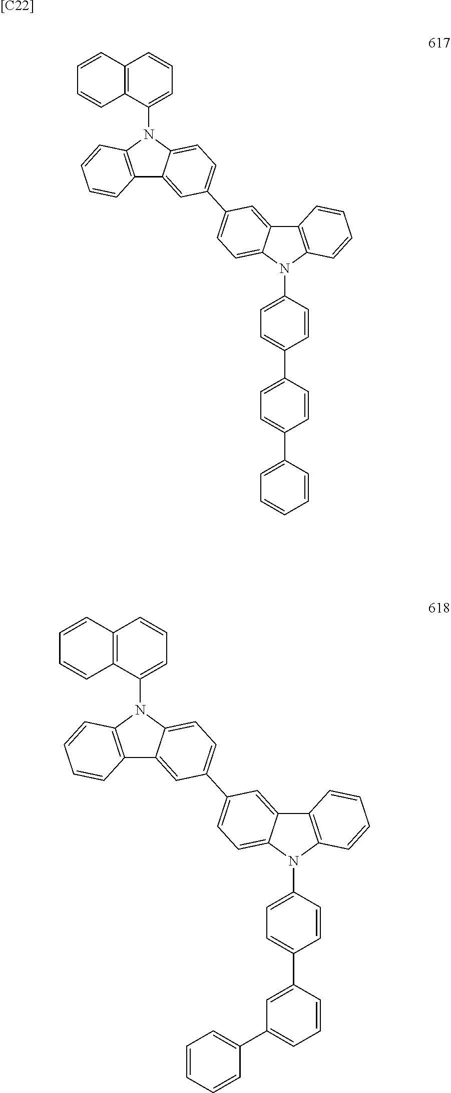

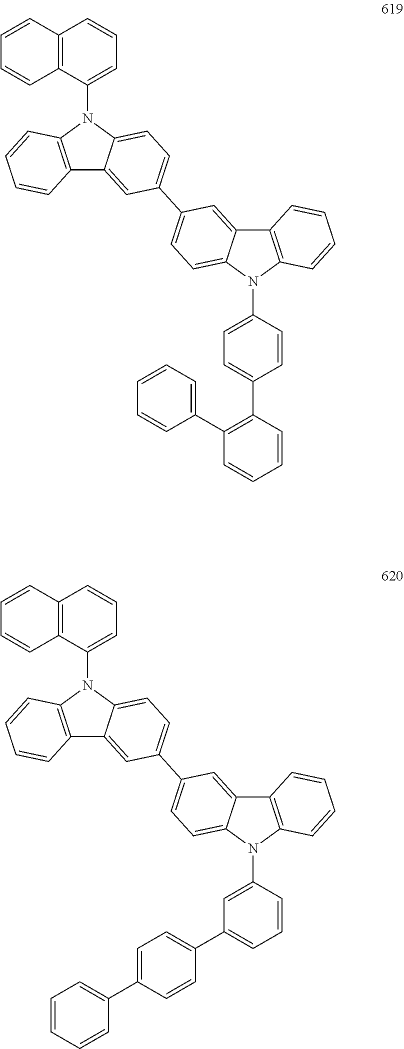

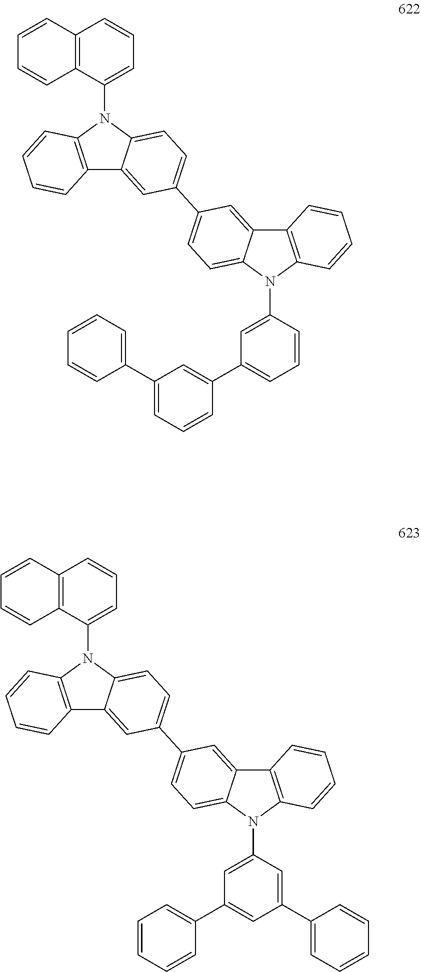

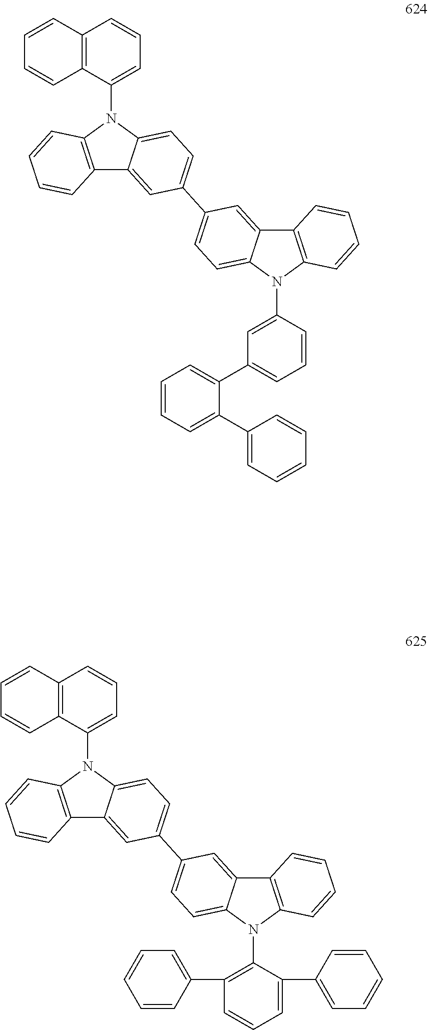

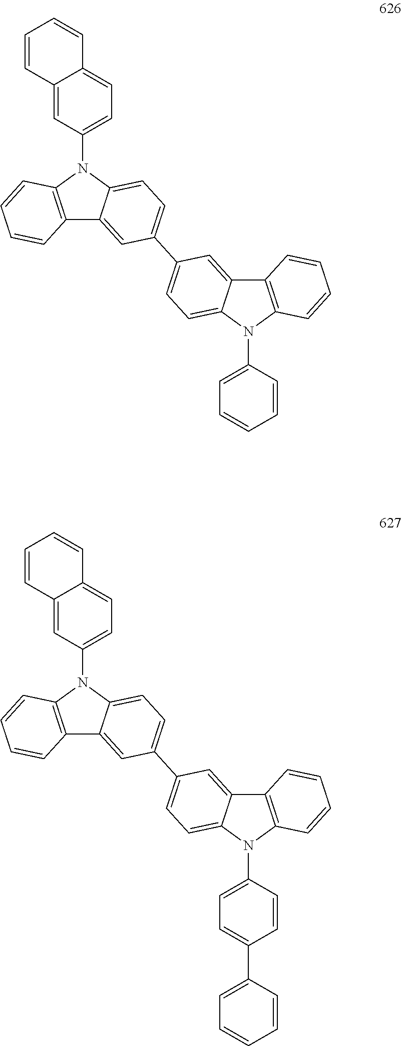

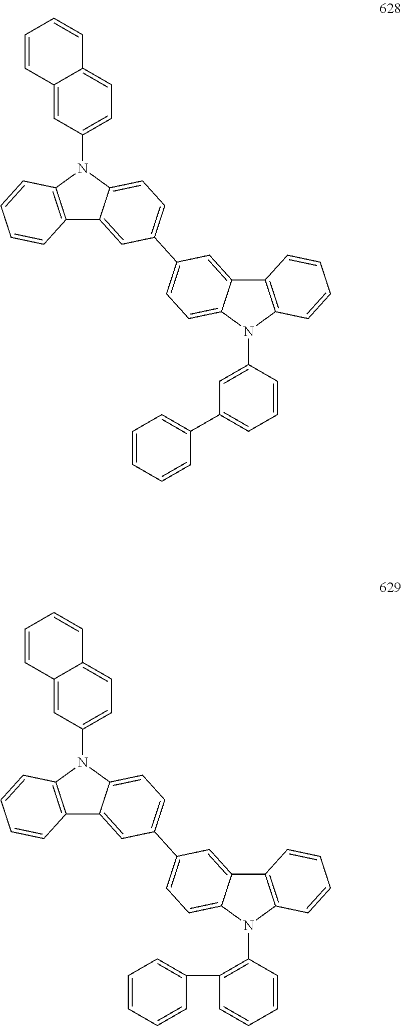

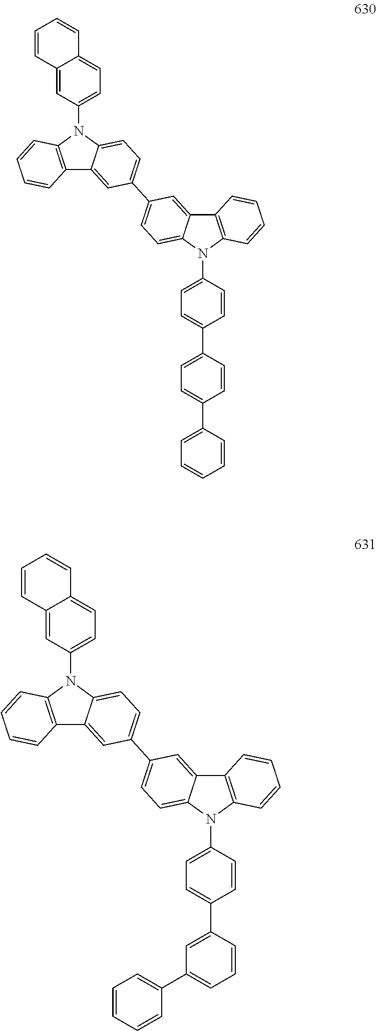

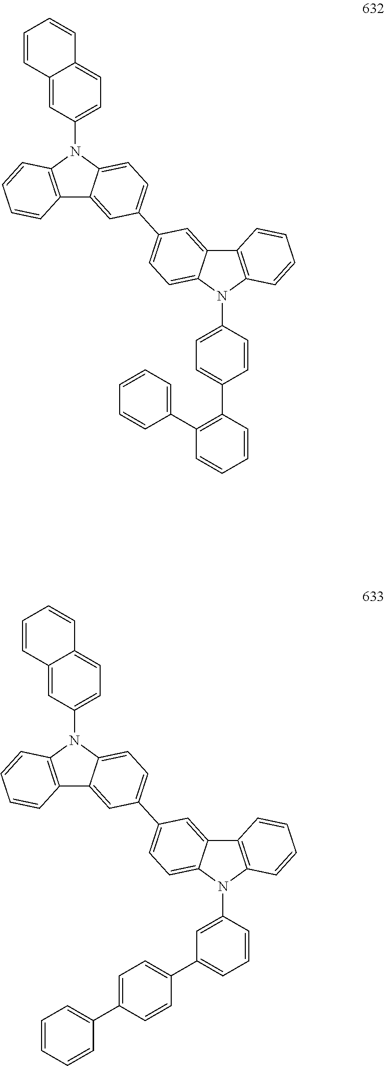

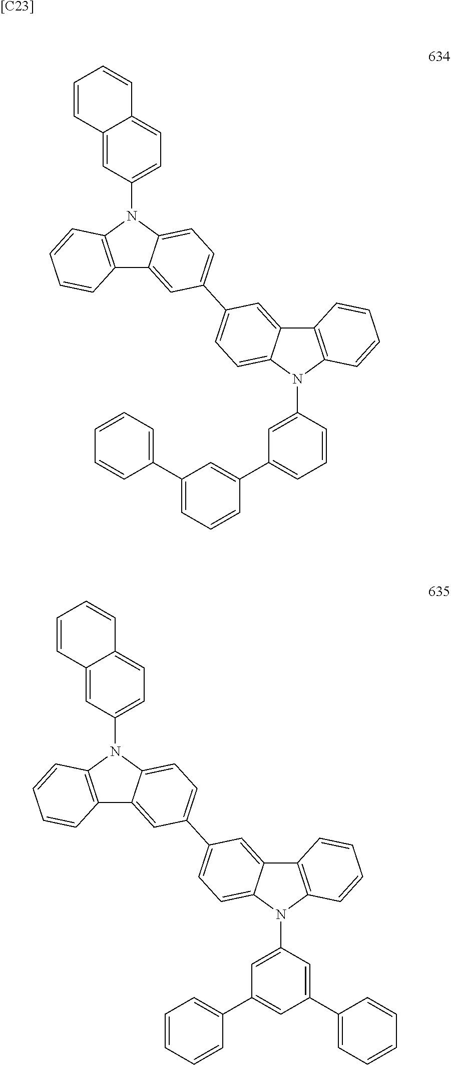

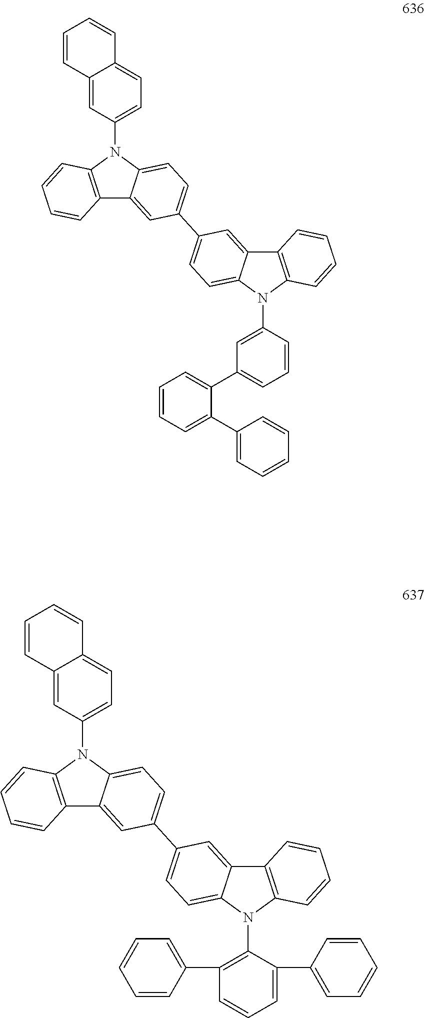

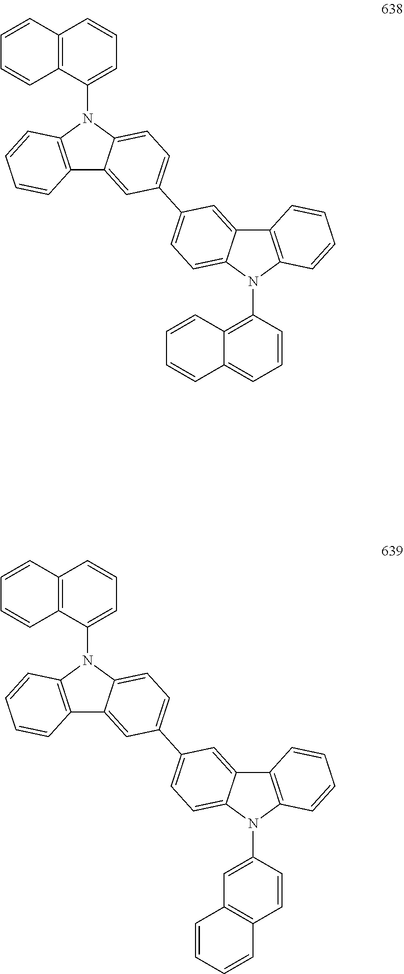

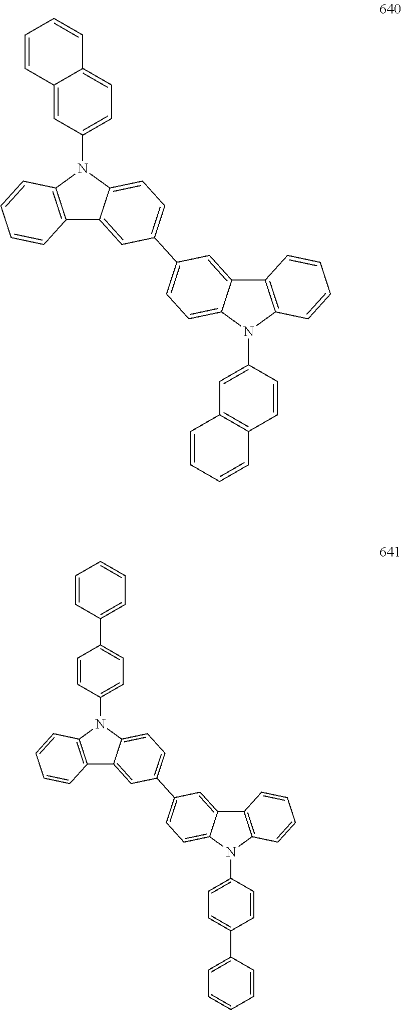

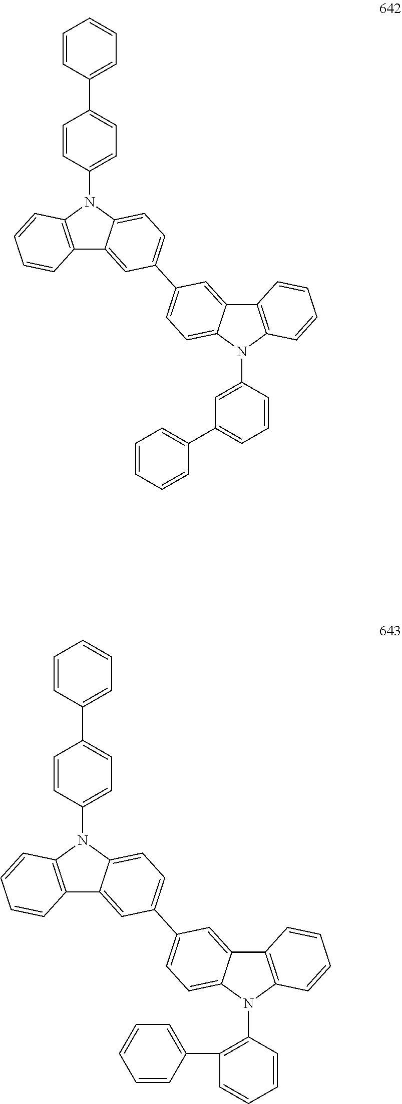

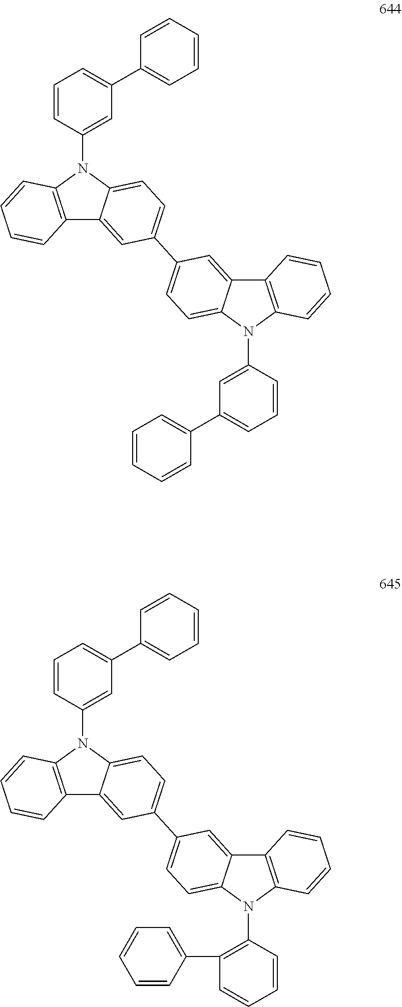

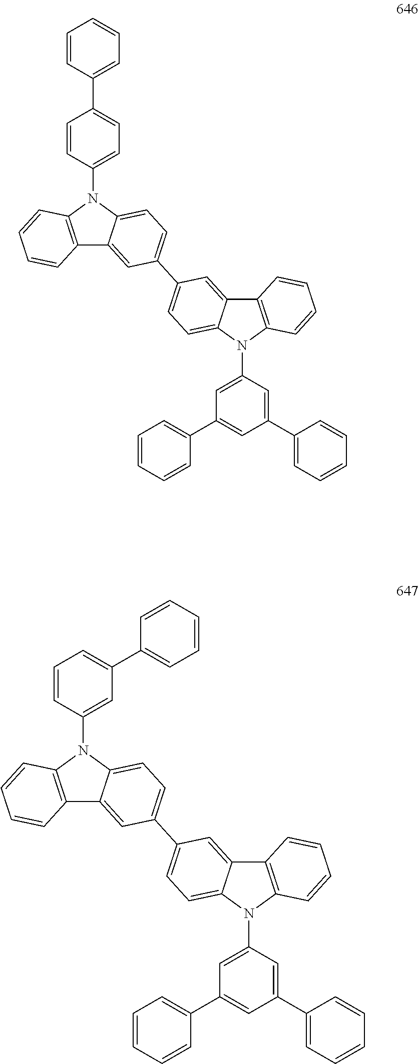

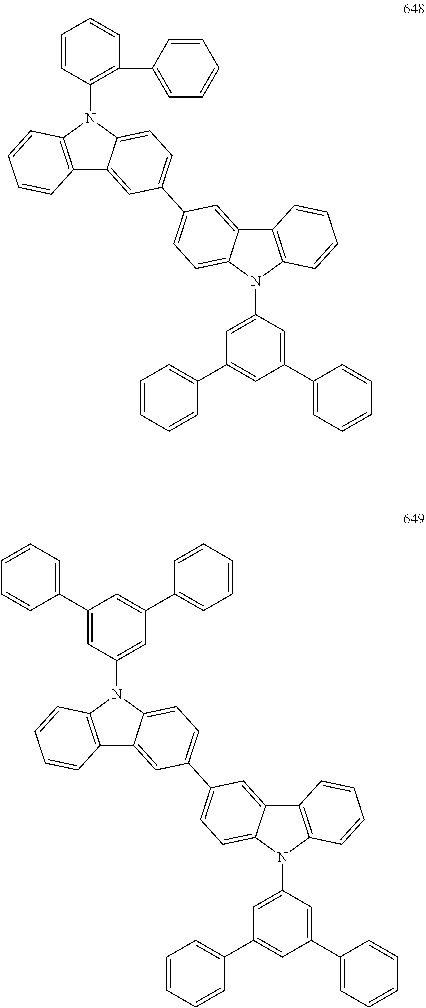

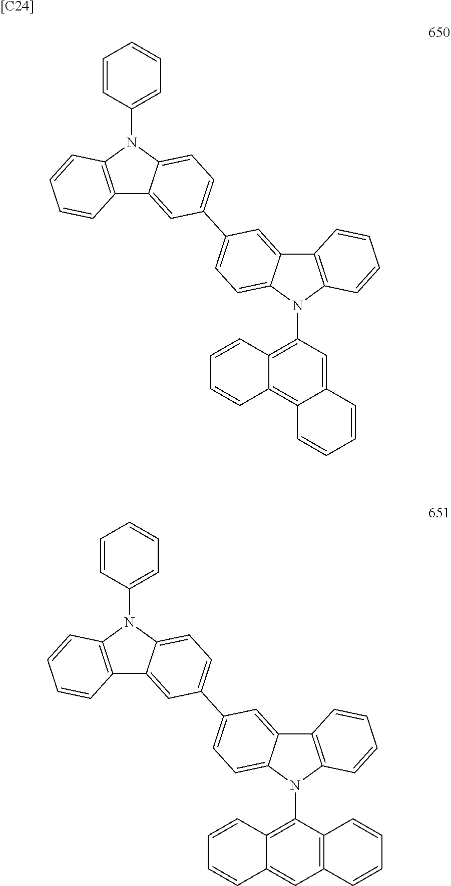

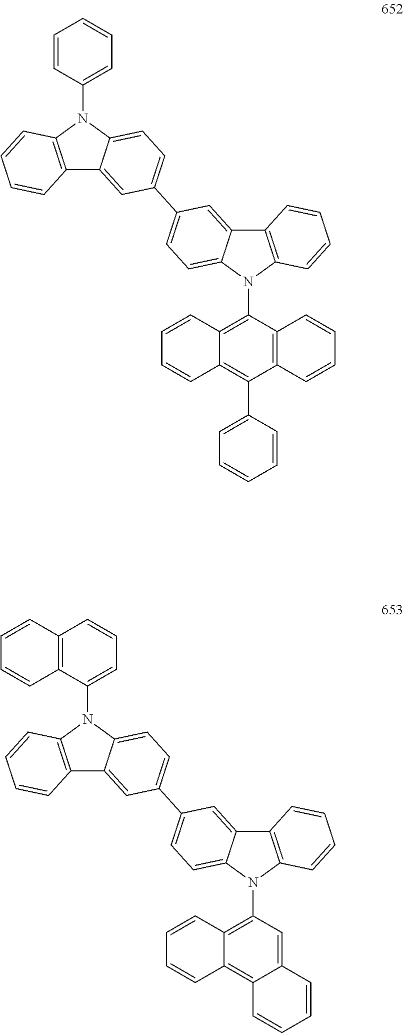

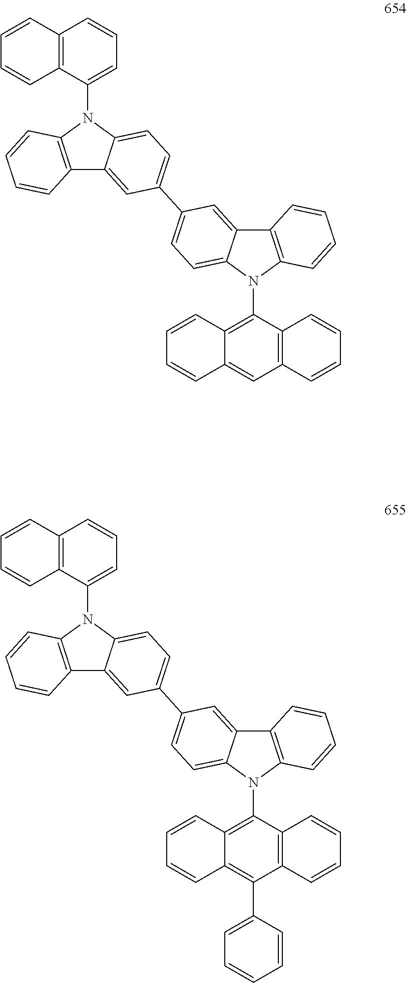

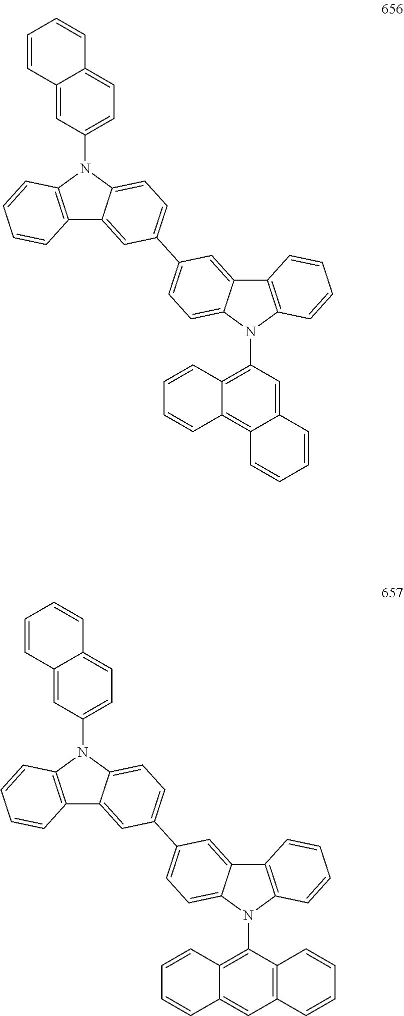

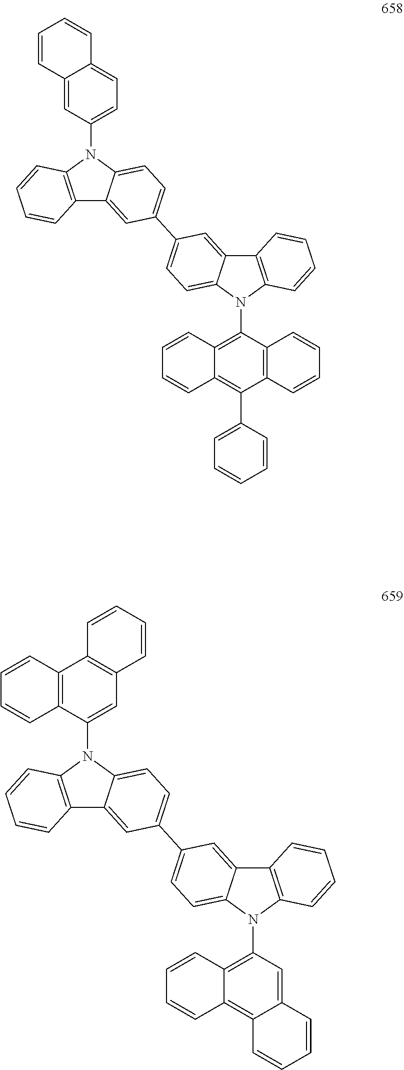

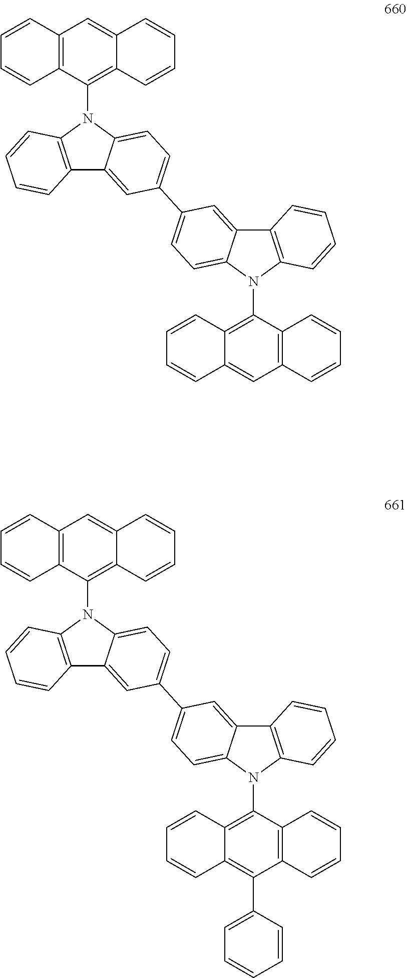

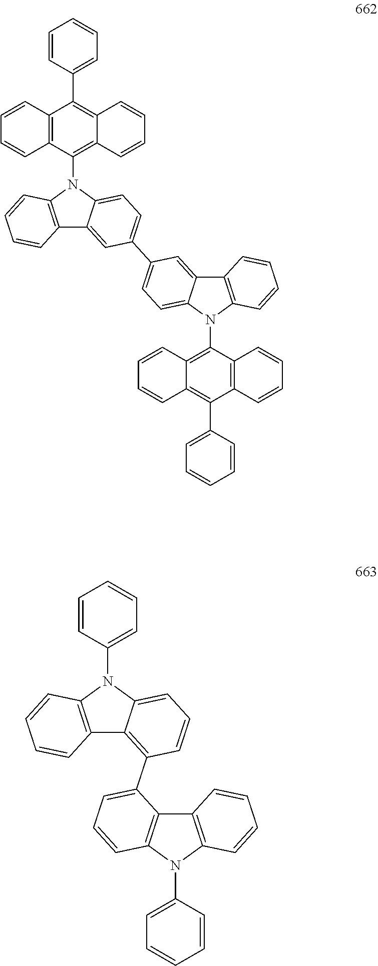

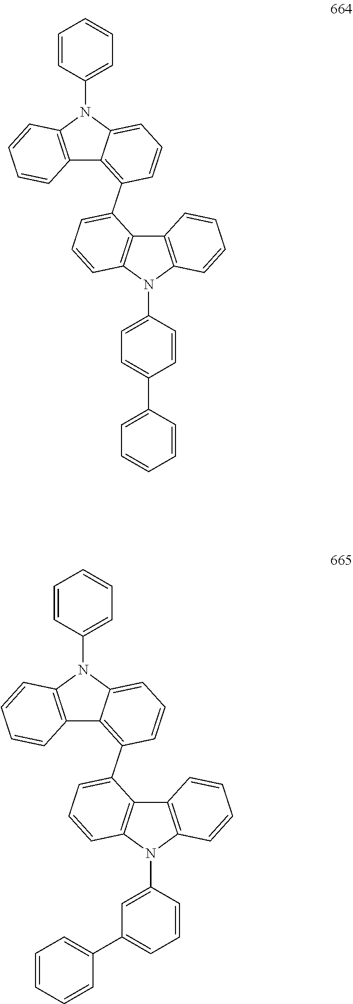

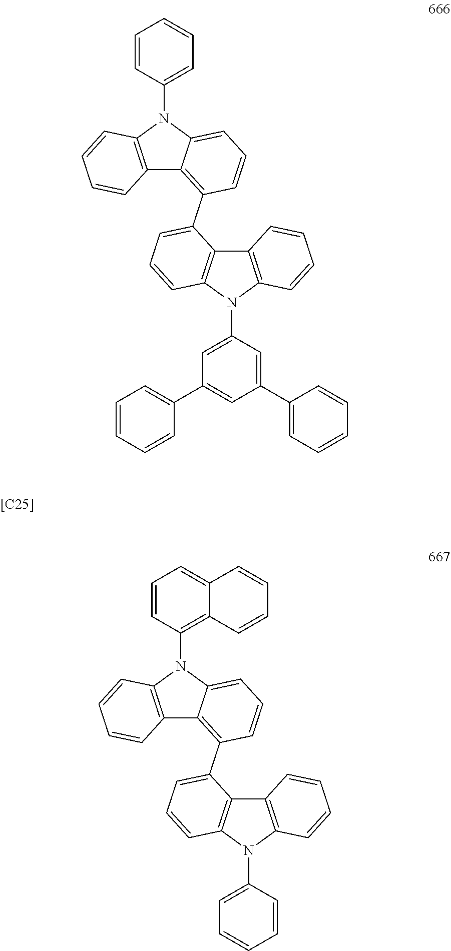

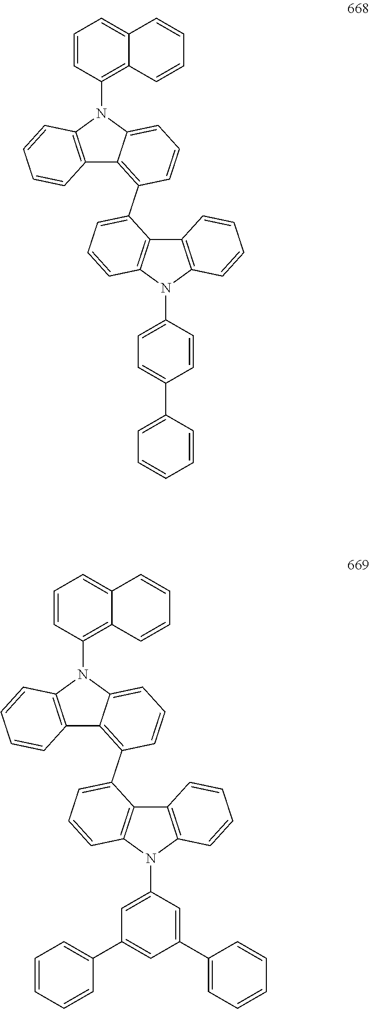

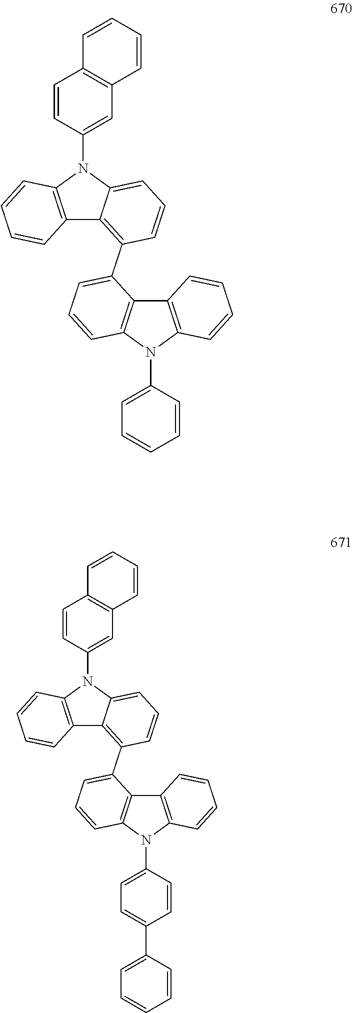

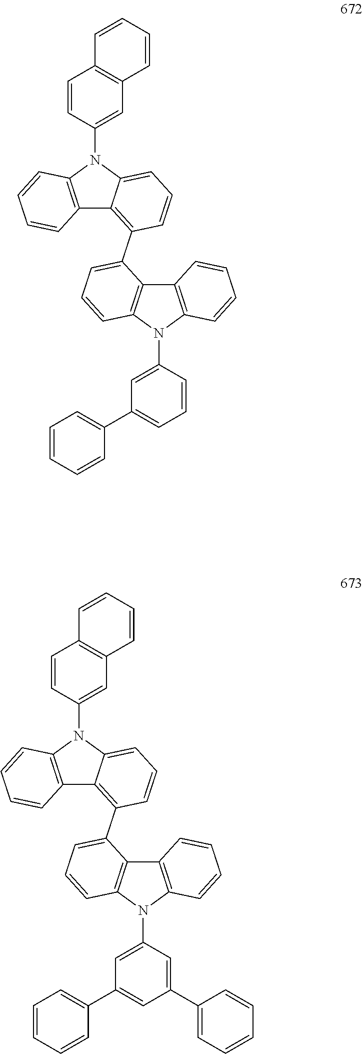

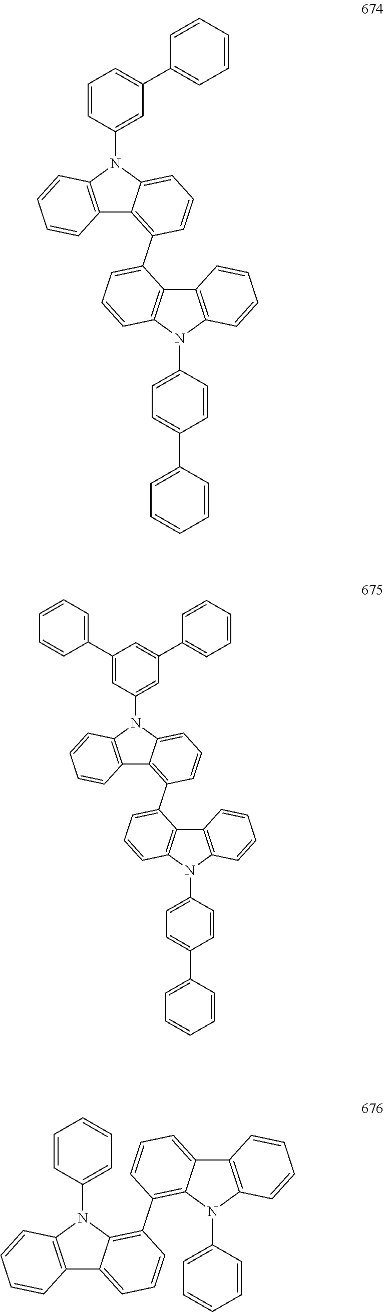

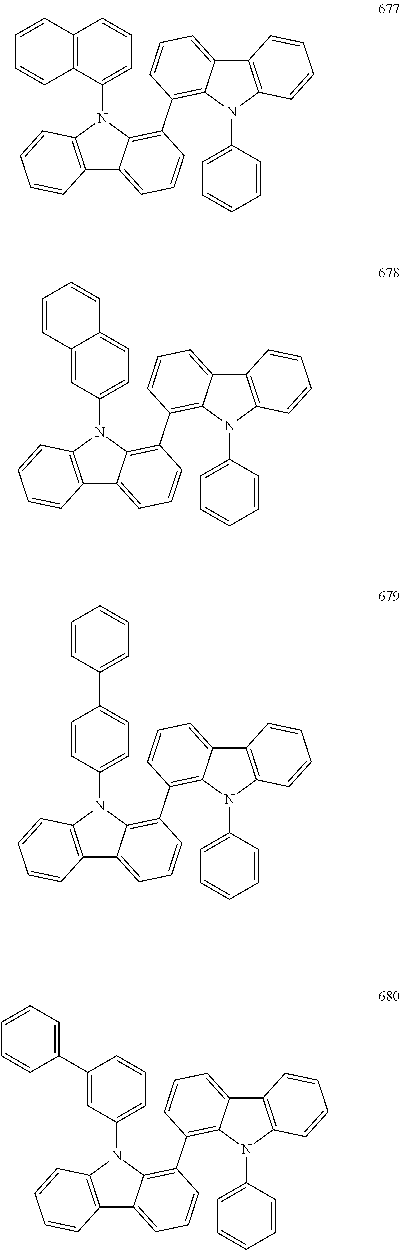

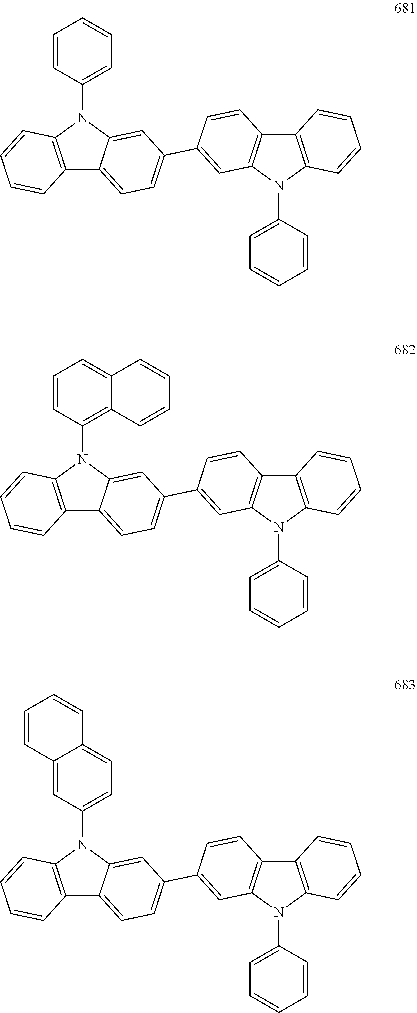

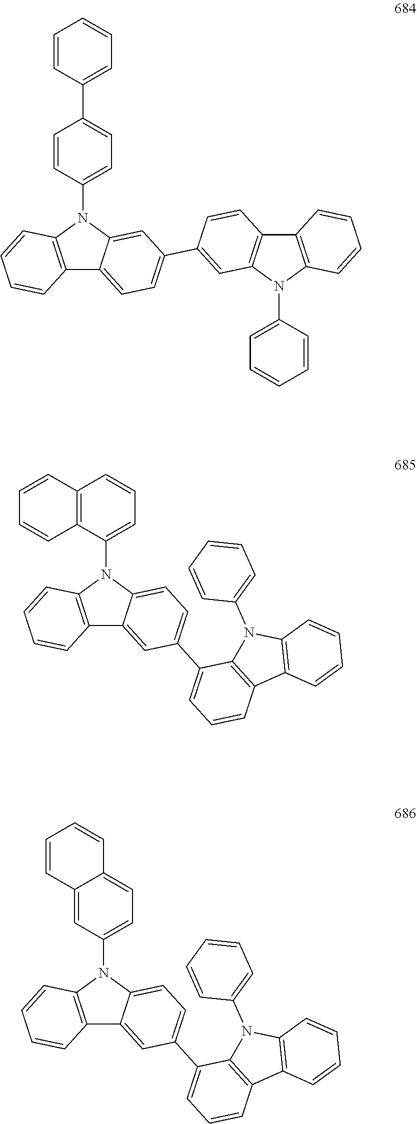

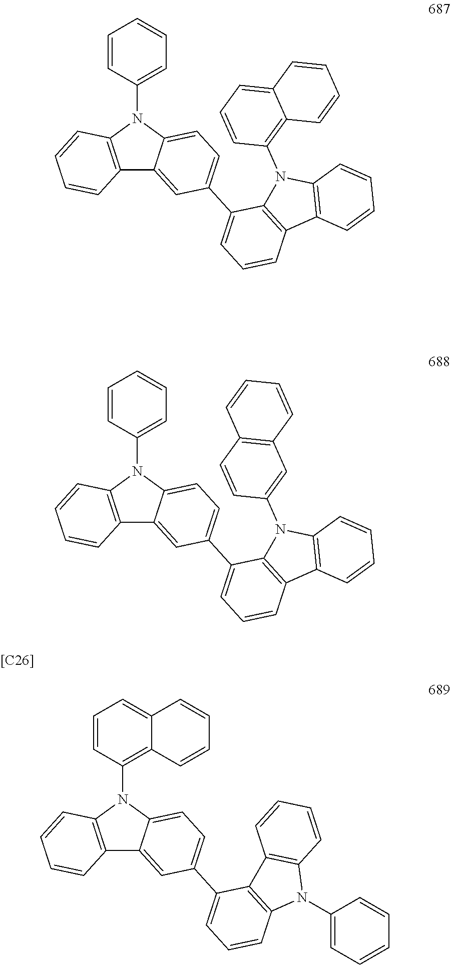

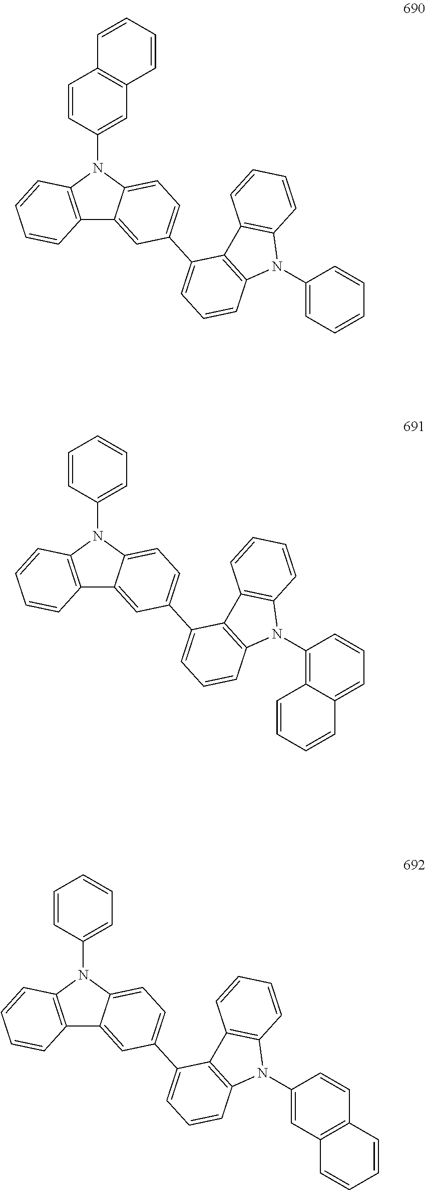

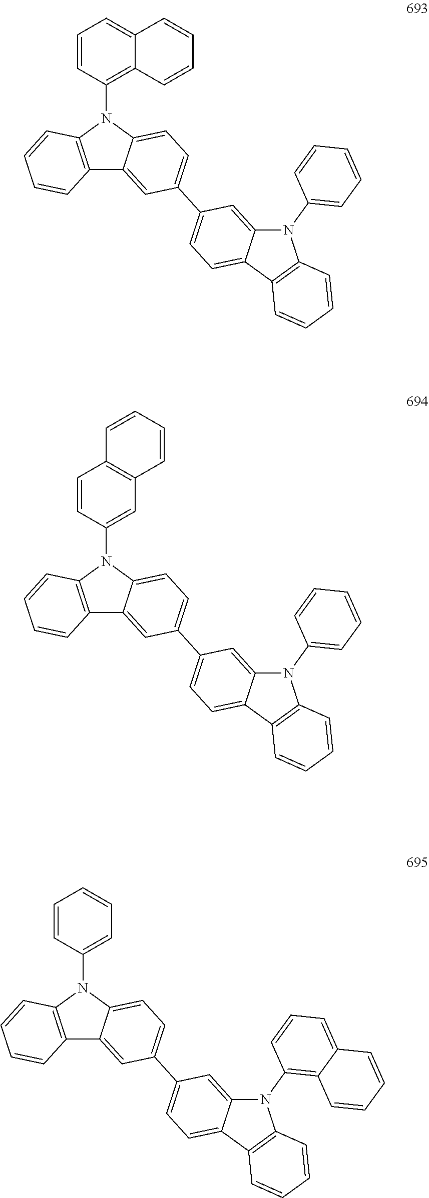

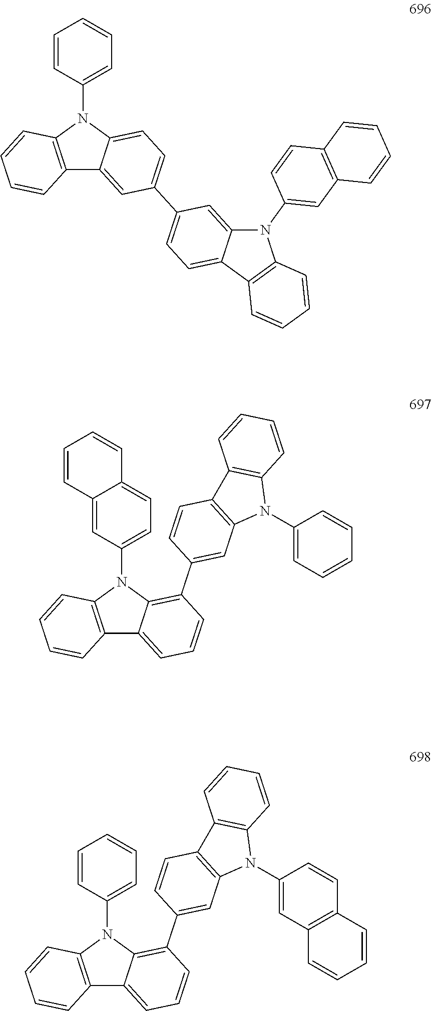

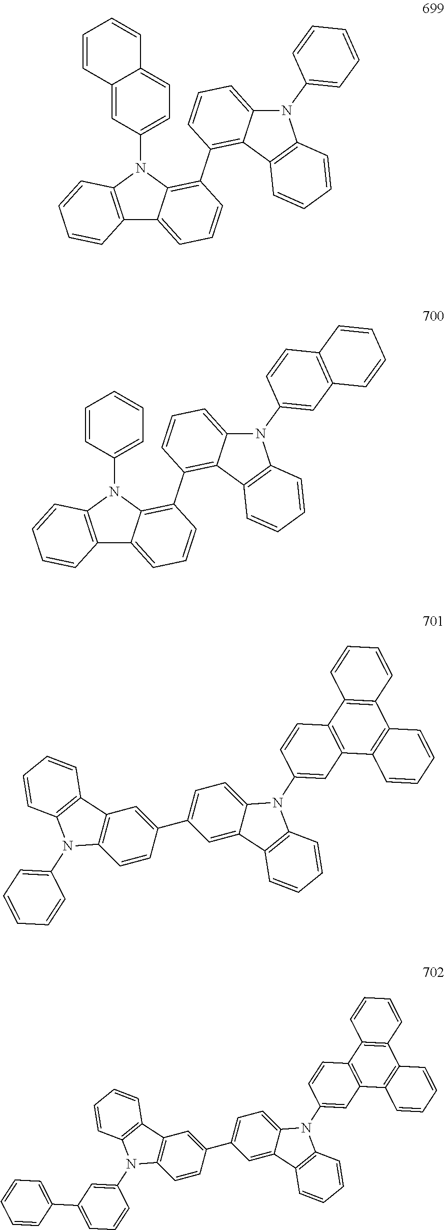

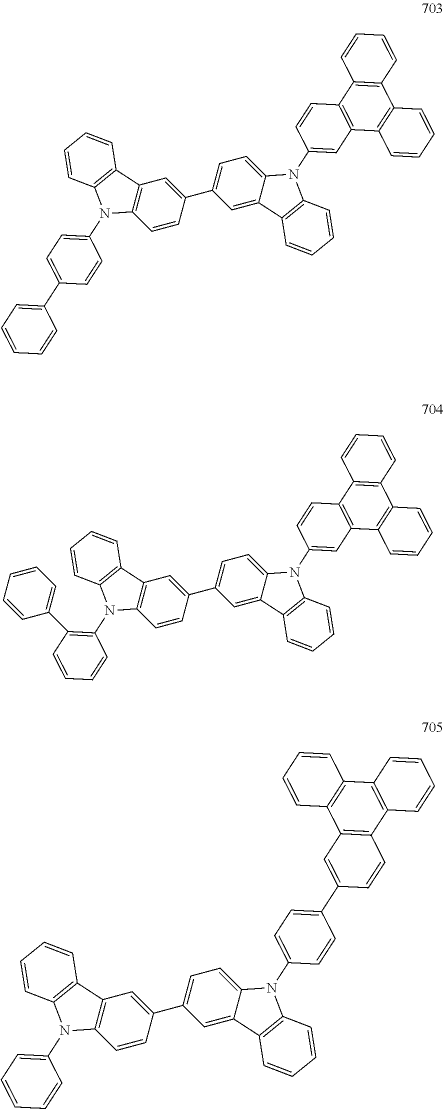

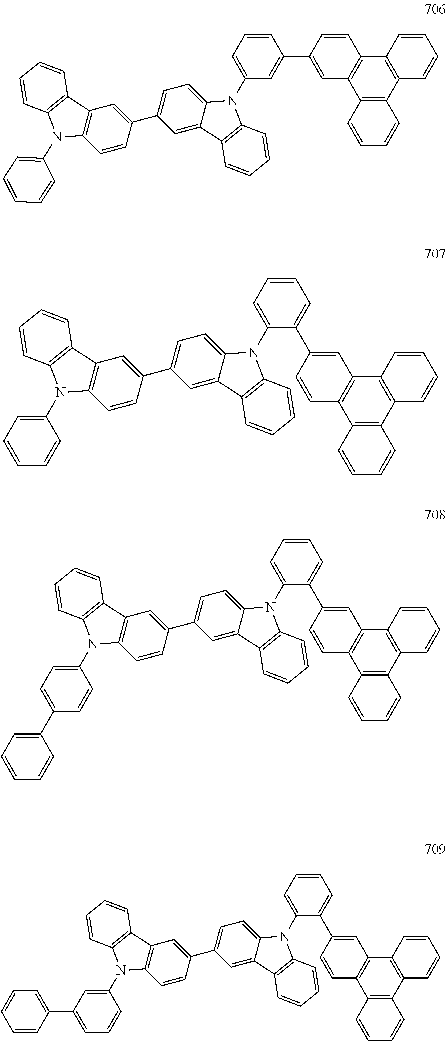

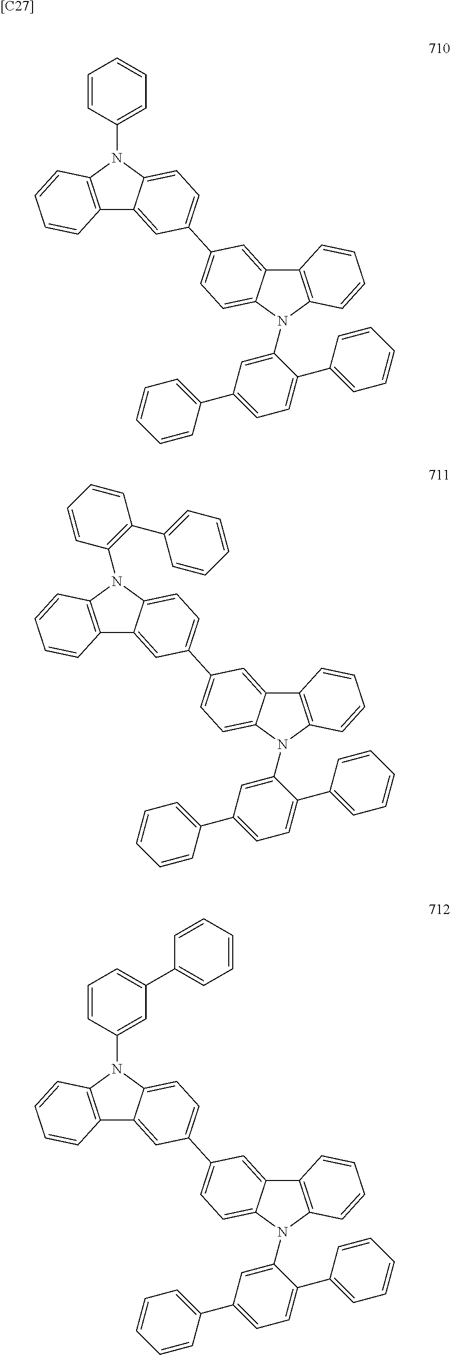

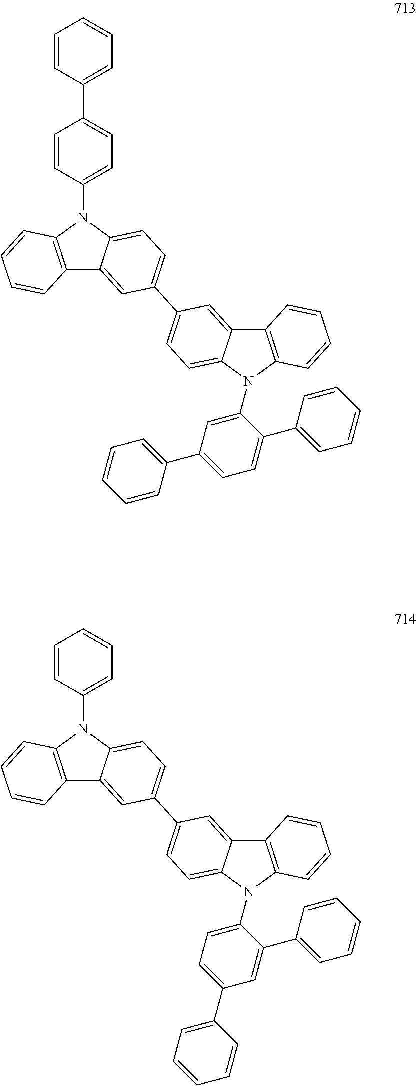

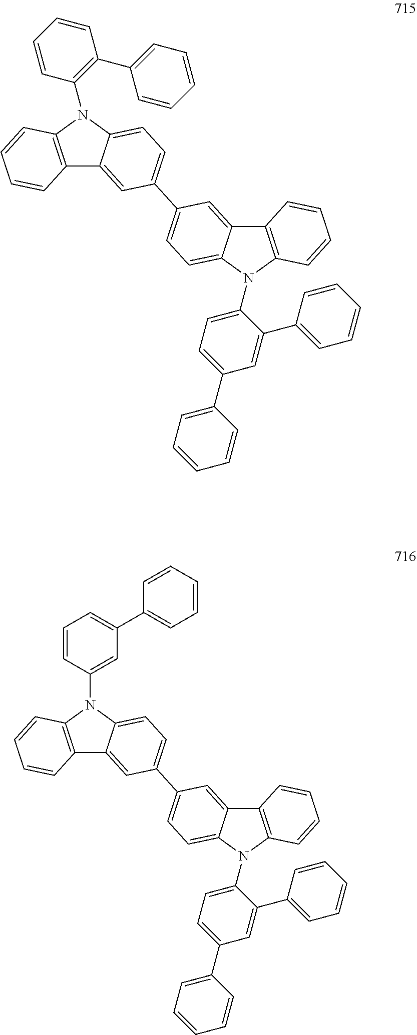

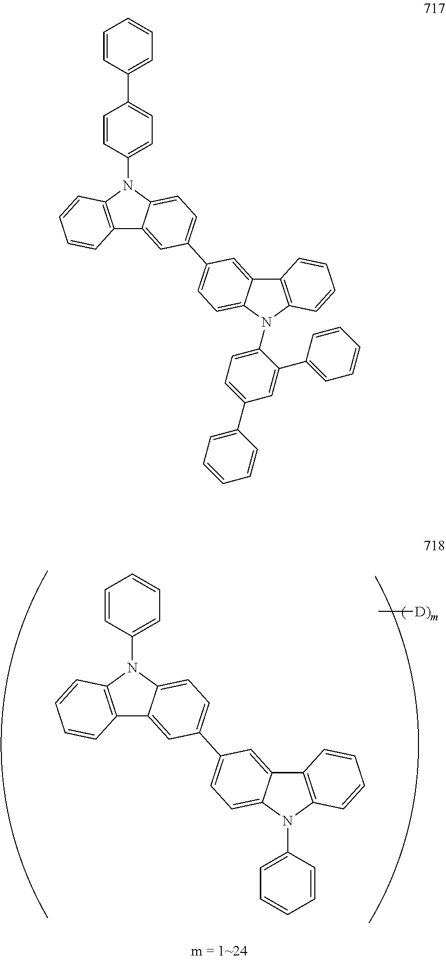

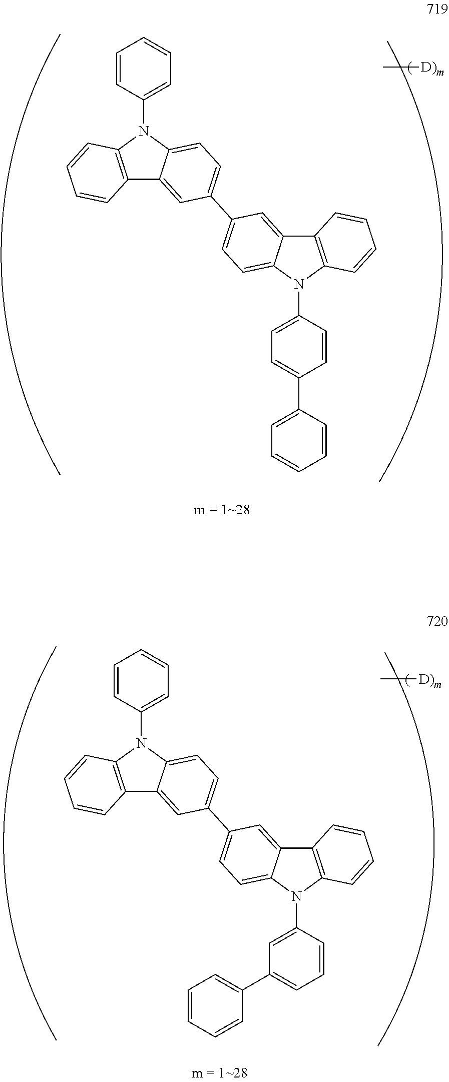

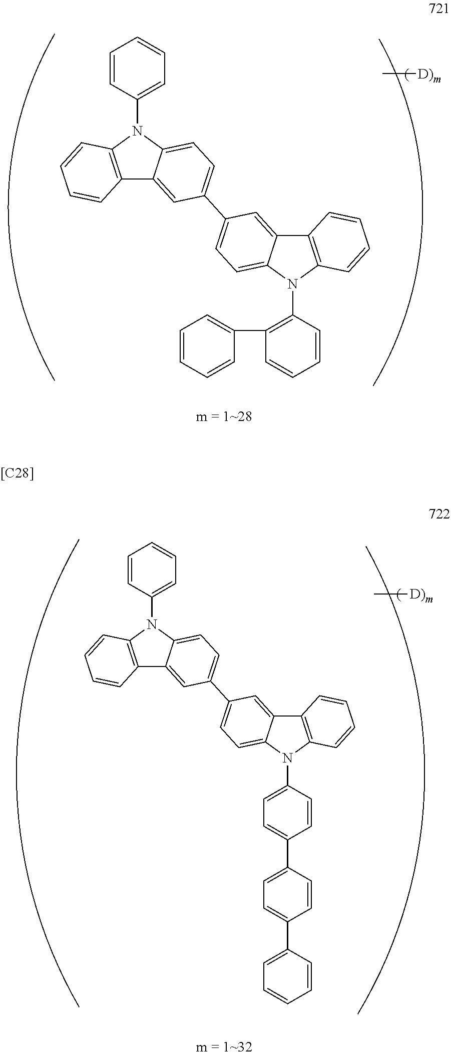

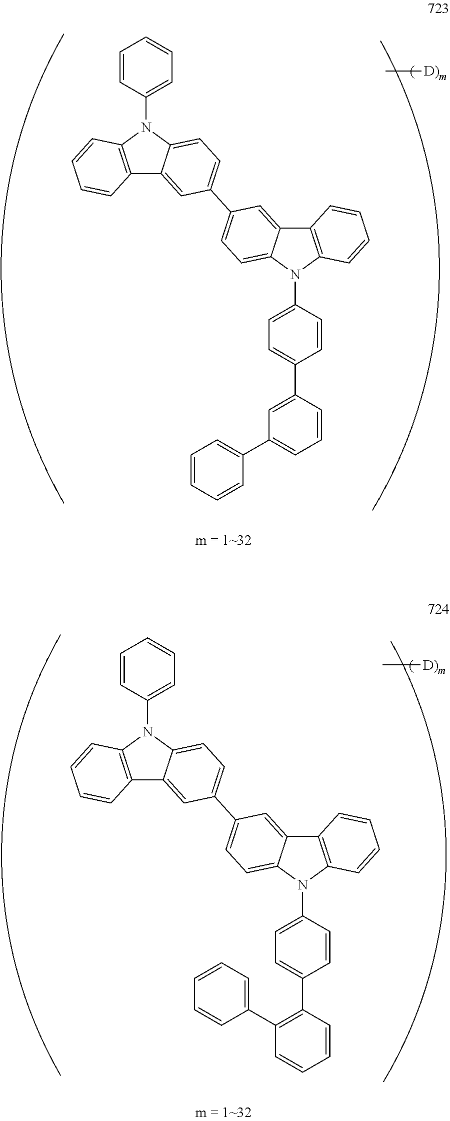

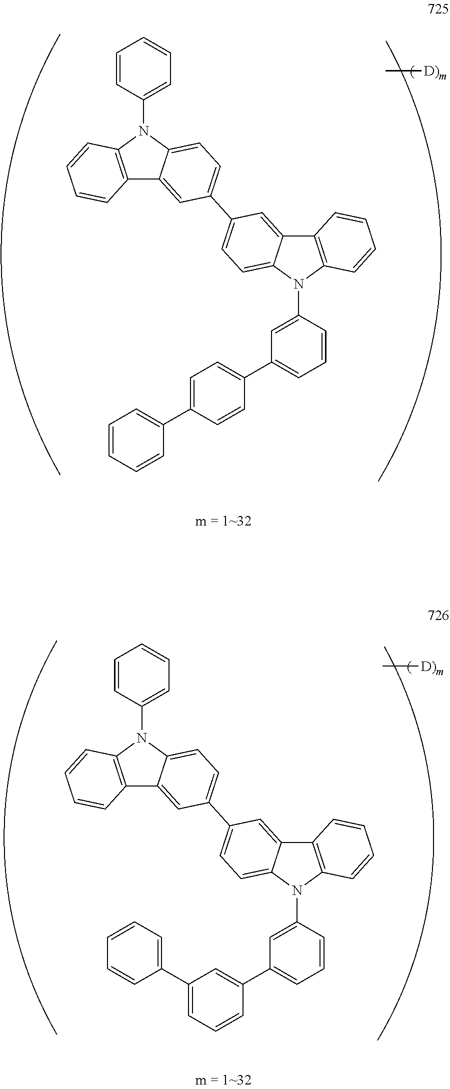

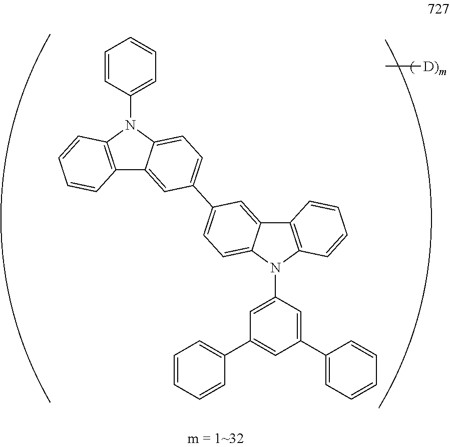

The host material for an organic EL device of the present invention is suitably used as a host material of a light-emitting layer.

Next, the structure of the organic EL device of the present invention will be described by referring to the drawing, but the structure of the organic EL device of the present invention is not limited thereto.

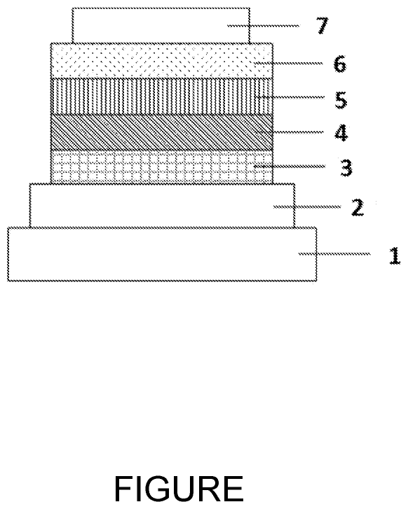

FIG. 1 is a cross-sectional view showing a structure example of an organic EL device generally used for the present invention, in which there are indicated a substrate 1, an anode 2, a hole injection layer 3, a hole transport layer 4, a light-emitting layer 5, an electron transport layer 6, and a cathode 7. The organic EL device of the present invention may have an exciton blocking layer adjacent to the light-emitting layer and may have an electron blocking layer between the light-emitting layer and the hole injection layer. The exciton blocking layer can be inserted into either of the anode side, and the cathode side of the light-emitting layer and inserted into both sides at the same time.

The organic EL device of the present invention has the anode, the light-emitting layer, and the cathode as essential layers, and preferably has a hole injection transport layer and an electron injection transport layer in addition to the essential layers, and further preferably has a hole blocking layer between the light-emitting layer and the electron injection transport layer. Note that the hole injection transport layer refers to either or both of a hole injection layer and a hole transport layer, and the electron injection transport layer refers to either or both of an electron injection layer and an electron transport layer.

A structure reverse to that of FIG. 1 is applicable, in which a cathode 7, an electron transport layer 6, a light-emitting layer 5, a hole transport layer 4, and an anode 2 are laminated on a substrate 1 in this order. In this case, layers may be added or omitted as necessary.

### Substrate

The organic EL device of the present invention is preferably supported on a substrate. The substrate is not particularly limited, and those conventionally used in organic EL devices may be used, and substrates made of, for example, glass, a transparent plastic, or quartz may be used.

### Anode

Regarding an anode material for an organic EL device, it is preferable to use a material of a metal, an alloy, an electrically conductive compound, and a mixture thereof, each having a large work function (4 eV or more). Specific examples of such an electrode material include a metal such as Au, and a conductive transparent material such as CuI, indium tin oxide (ITO), SnO₂, and ZnO. In addition, an amorphous material such as IDIXO (In₂O₃—ZnO), which is capable of forming a transparent conductive film, may be used. Regarding the anode, such an electrode material is used to form a thin film by, for example, a vapor-deposition or sputtering method, and a desired shape pattern may be formed by a photolithographic method; or if the pattern accuracy is not particularly required (about 100 μm or more), a pattern may be formed via a desired shape mask when the electrode material is vapor-deposited or sputtered. Alternatively, when a coatable substance such as an organic conductive compound is used, a wet film formation method such as a printing method or a coating method may be used. For taking emitted light from the anode, it is desired to have a transmittance of more than 10%, and the sheet resistance for the anode is preferably several hundreds Ω/□ or less. The film thickness is selected usually within 10 to 1000 nm, preferably within 10 to 200 nm though depending on the material.

### Cathode

Meanwhile, regarding a cathode material, a material of a metal (an electron injection metal), an alloy, an electrically conductive compound, or a mixture thereof, each having a small work function (4 eV or less) is used. Specific examples of such an electrode material include sodium, a sodium-potassium alloy, magnesium, lithium, a magnesium/copper mixture, a magnesium/silver mixture, a magnesium/aluminum mixture, a magnesium/indium mixture, an aluminum/aluminum oxide (Al₂O₃) mixture, indium, a lithium/aluminum mixture, and a rare earth metal. Among these, from the viewpoint of the electron injectability and the durability against oxidation and the like, a mixture of an electron injection metal and a second metal which is a stable metal having a larger work function value is suitable, and examples thereof include a magnesium/silver mixture, a magnesium/aluminum mixture, a magnesium/indium mixture, an aluminum/aluminum oxide mixture, a lithium/aluminum mixture and aluminum. The cathode can be produced by forming a thin film by a method such as vapor-depositing or sputtering of such a cathode material. In addition, the sheet resistance of cathode is preferably several hundreds Ω/□ or less. The film thickness is selected usually within 10 nm to 5 μm, preferably within 50 to 200 nm. Note that for transmission of emitted light, if either one of the anode and cathode of the organic EL device is transparent or translucent, emission luminance is improved, which is convenient.

In addition, formation of a film of the above metal with a thickness of 1 to 20 nm, on the cathode, followed by formation of a conductive transparent material described in the description on the anode thereon, enables production of a transparent or translucent cathode, and application of this enables production of a device wherein an anode and a cathode both have transmittance.

### Light-Emitting Layer

The light-emitting layer is a layer that emits light after excitons are generated when holes and electrons injected from the anode and the cathode, respectively, are recombined. As a light-emitting layer, an organic light-emitting dopant material and a host material may be contained.

As a host, a host material represented by any of the general formulas (1) to (5) (also referred to as the host material of the present invention.) is used.

As the host material of the present invention, one kind thereof may be used, or two or more kinds of different compounds may be used, or one, or two or more other host materials such as known host materials may be used in combination. As other host material, a compound is preferable, which has hole transport ability and electron transport ability, which prevents elongation of the wavelength of light emitted, and which also has a high glass transition temperature.

When the host material of the present invention is contained as the first host material, the compound represented by the general formula (10) is particularly preferably used as the second host material, or other host material shown below may also be used as the second host material. When the host material of the present invention is used as the first host material and the compound represented by the general formula (10) is used as the second host material, other host material may also be used as a third host material.

Such other host material is one known in many Patent Literatures and the like, and can be selected therefrom. Specific examples of the host material include, but not particularly limited thereto, indolocarbazole derivatives described in WO2008/056746A1 or WO2008/146839A1, carbazole derivatives described in WO2009/086028A1 or WO2012/077520A1, CBP (N,N-biscarbazolylbiphenyl) derivatives, triazine derivatives described in WO2014/185595A1 or WO2018/021663A1, indenocarbazole derivatives described in WO2010/136109A1 or WO2011/000455A1, dibenzofuran derivatives described in WO2015/169412A1, triazole derivatives, indole derivatives, oxazole derivatives, oxadiazole derivatives, imidazole derivatives, polyarylalkane derivatives, pyrazoline derivatives, pyrazolone derivatives, phenylenediamine derivatives, arylamine derivatives, amino-substituted chalcone derivatives, styrylanthracene derivatives, fluorenone derivatives, hydrazone derivatives, stilbene derivatives, silazane derivatives, aromatic tert-amine compounds, styrylamine compounds, aromatic dimethylidene-based compounds, porphyrin-based compounds, anthraquinodimethane derivatives, anthrone derivatives, diphenylquinone derivatives, thiopyran dioxide derivatives, heterocyclic tetracarboxylic anhydrides of naphthalene, perylene, or the like, various metal complexes typified by phthalocyanine derivatives, metal complexes of 8-quinolinol derivatives, metal phthalocyanine, and metal complexes of benzoxazole or benzothiazole derivatives, and polymer compounds such as polysilane-based compounds, poly(N-vinylcarbazole) derivatives, aniline-based copolymers, thiophene oligomers, polythiophene derivatives, polyphenylene derivatives, polyphenylene vinylene derivatives, and polyfluorene derivatives.

Specific examples of other host material described above are shown below, but are not limited thereto.

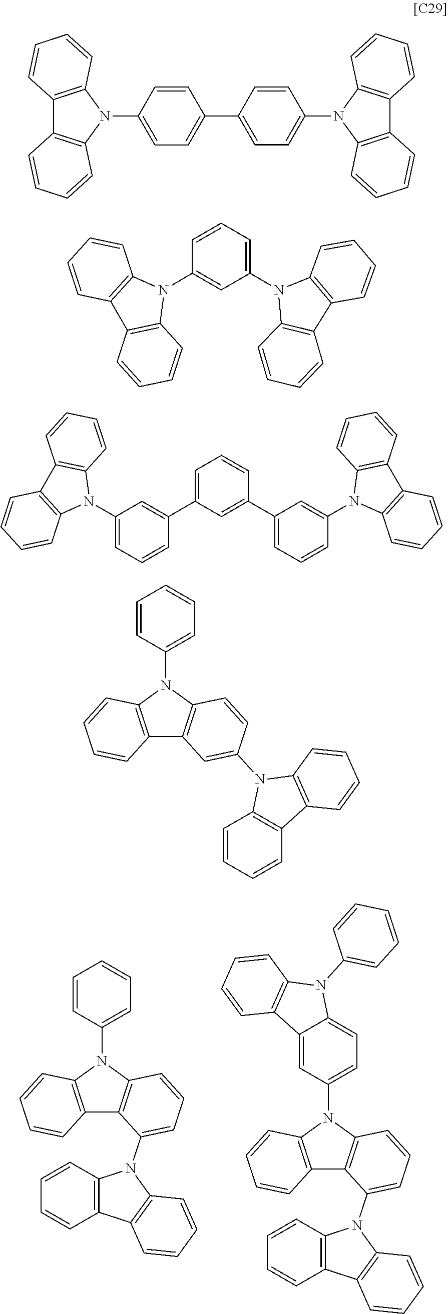

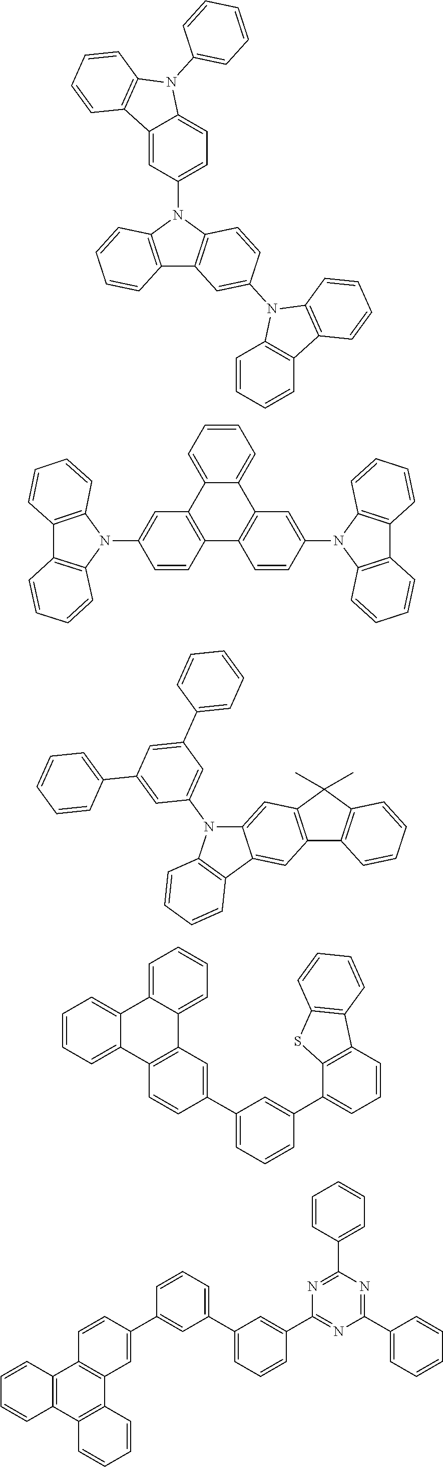

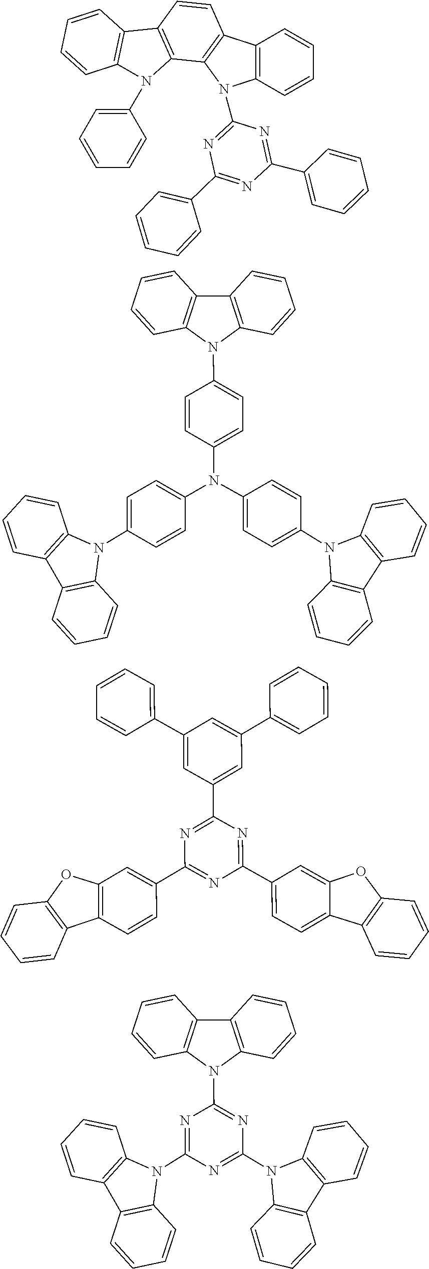

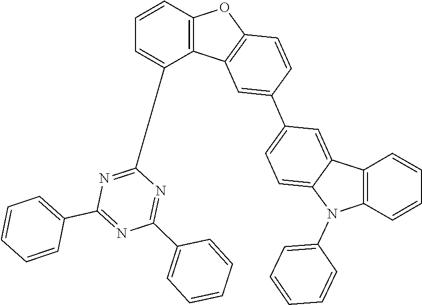 [SMILES: c1ccc(-c2nc(-c3ccccc3)nc(-c3cccc4oc5ccc(-c6ccc7c(c6)c6ccccc6n7-c6ccccc6)cc5c34)n2)cc1]

Preferred examples of the organic light-emitting dopant material include a phosphorescent dopant, a fluorescence-emitting dopant or a thermally activated delayed fluorescence-emitting dopant.

Preferred is a phosphorescent dopant including an organic metal complex containing at least one metal selected from ruthenium, rhodium, palladium, silver, rhenium, osmium, iridium, platinum and gold. Specifically, iridium complexes described in J. Am. Chem. Soc. 2001, 123, 4304, JP2013-530515A, US2016/0049599A1, US2017/0069848A1, US2018/0282356A1, US2019/0036043A1, and the like, or platinum complexes described in US2018/0013078A1, KR2018/094482A, and the like are preferably used, but the phosphorescent dopant material is not limited thereto.

Regarding the phosphorescent dopant material, only one kind thereof may be contained in the light-emitting layer, or two or more kinds thereof may be contained. A content of the phosphorescent dopant material is preferably 0.1 to 30 wt % and more preferably 1 to 20 wt % with respect to the host material.

The phosphorescent dopant material is not particularly limited, and specific examples thereof include the following.

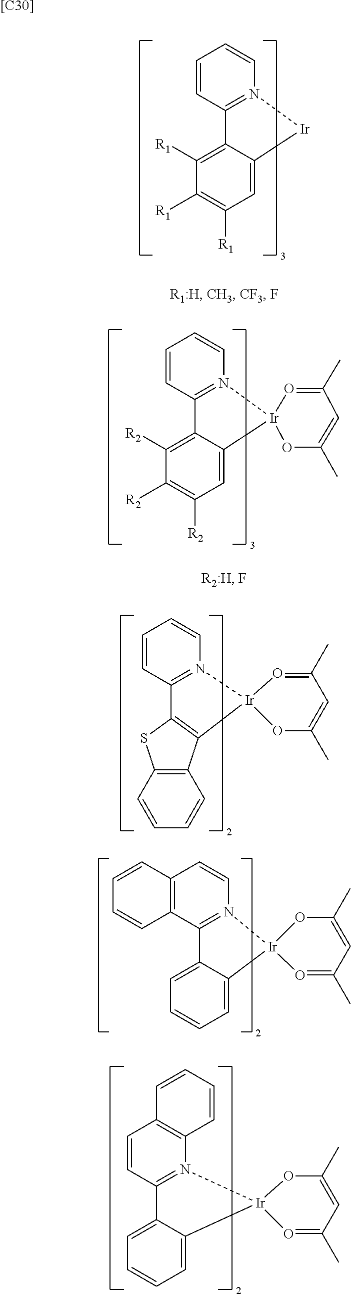

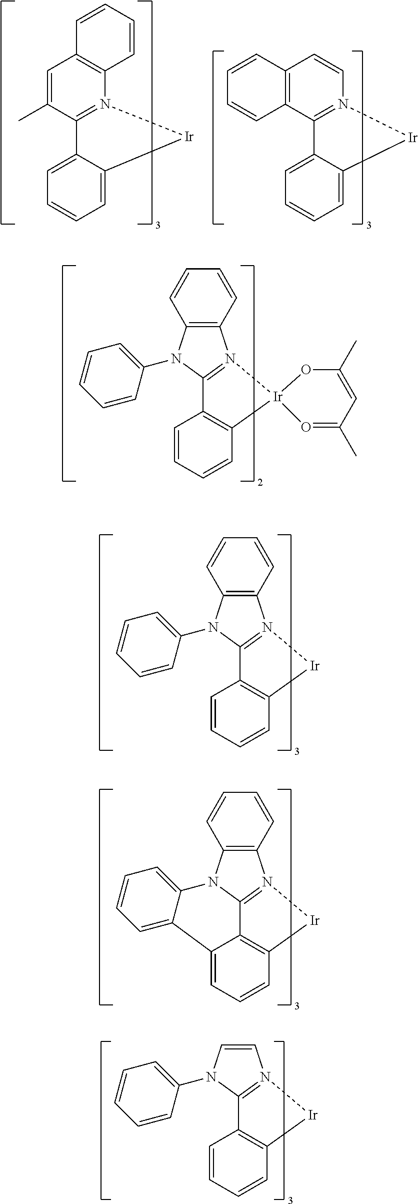

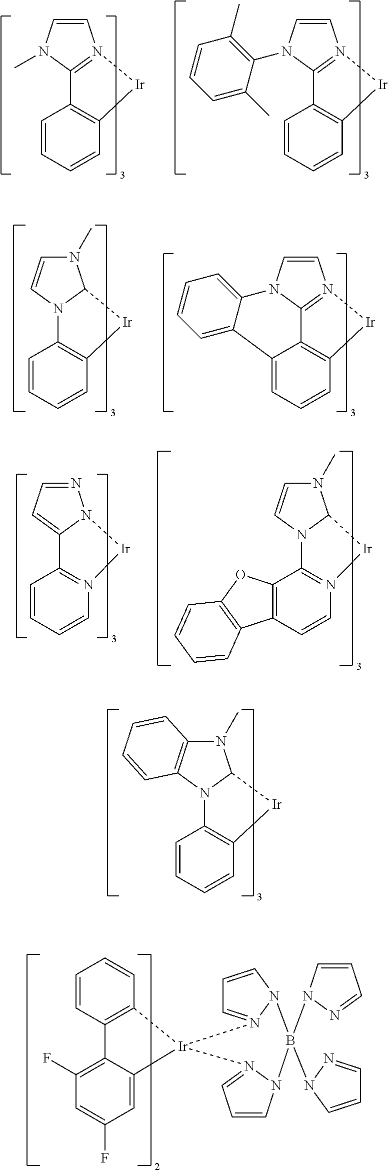

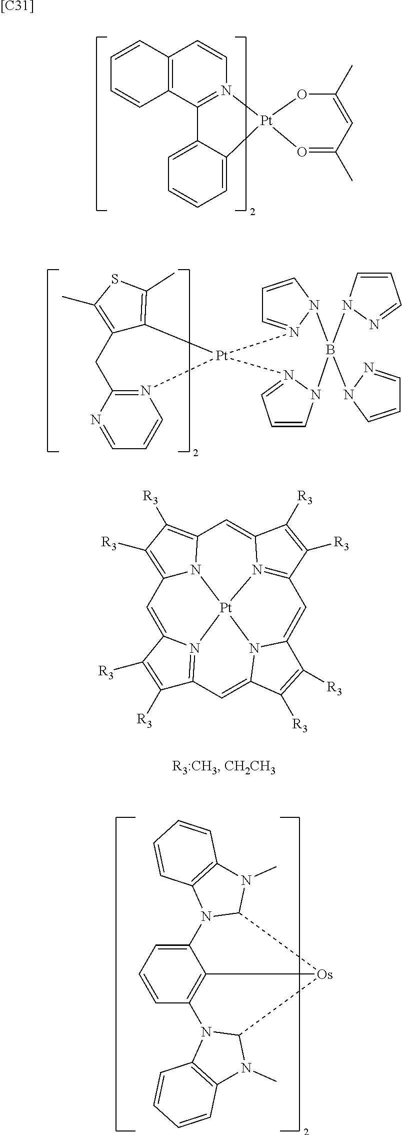

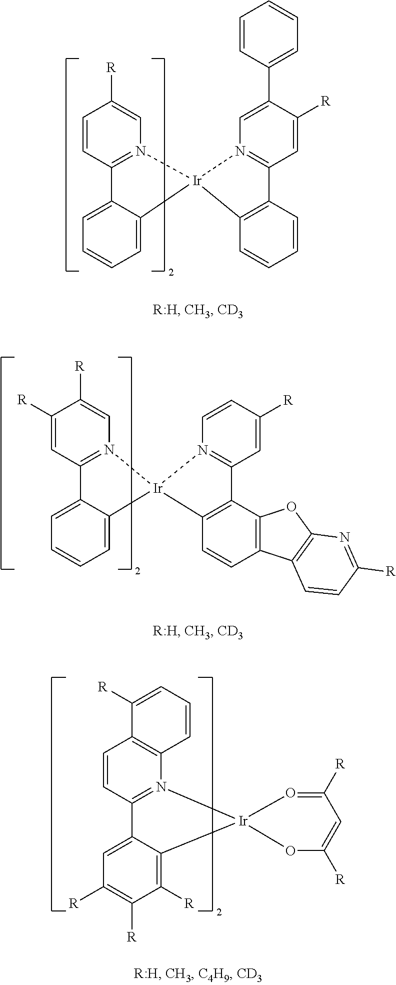

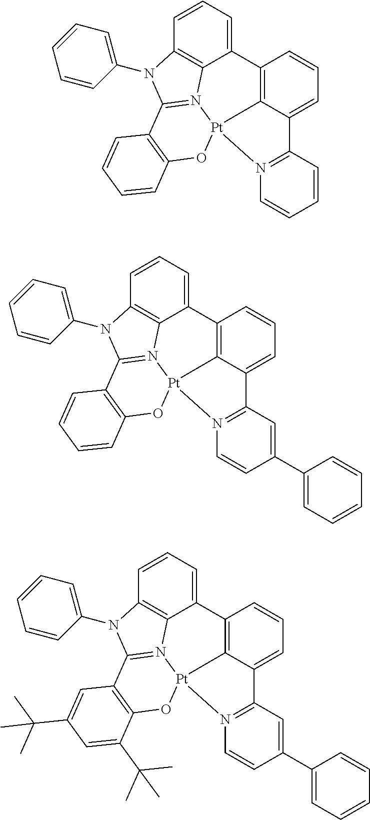

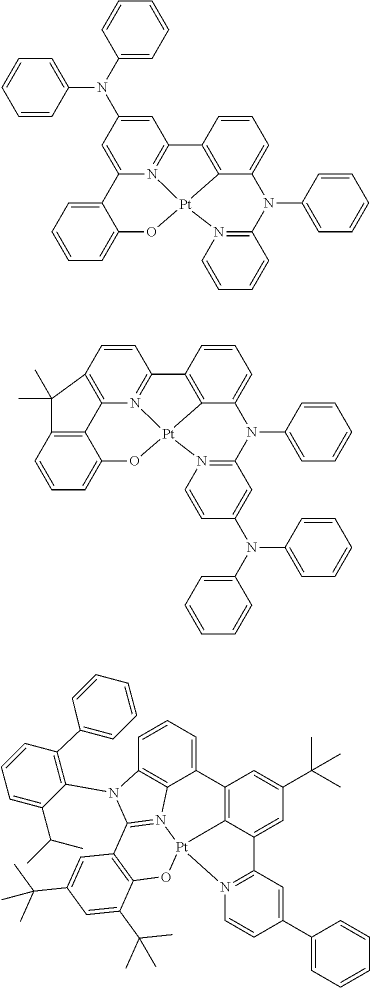

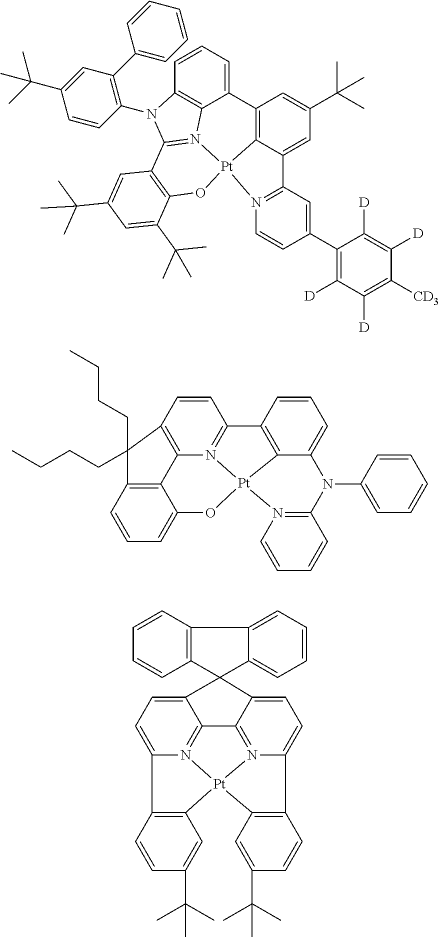

The fluorescence-emitting dopant is not particularly limited. Examples thereof include benzoxazole derivatives, benzothiazole derivatives, benzimidazole derivatives, styrylbenzene derivatives, polyphenyl derivatives, diphenylbutadiene derivatives, tetraphenyl butadiene derivatives, naphthalimido derivatives, coumarin derivatives, fused aromatic compounds, perinone derivatives, oxadiazole derivatives, oxazine derivatives, aldazine derivatives, pyrrolidine derivatives, cyclopentadiene derivatives, bisstyryl anthracene derivatives, quinacridone derivatives, pyrrolopyridine derivatives, thiadiazolopyridine derivatives, styrylamine derivatives, diketopyrrolopyrrole derivatives, aromatic dimethylidine compounds, metal complexes of 8-quinolinol derivatives or metal complexes of pyromethene derivatives, rare earth complexes, various metal complexes represented by transition metal complexes, polymer compounds such as polythiophene, polyphenylene, and polyphenylene vinylene, and organosilane derivatives. Preferred examples thereof include fused aromatic derivatives, styryl derivatives, diketopyrrolopyrrole derivatives, oxazine derivatives, pyromethene metal complexes, transition metal complexes, and lanthanoid complexes. More preferable examples thereof include naphthalene, pyrene, chrysene, triphenylene, benzo[c]phenanthrene, benzo[a]anthracene, pentacene, perylene, fluoranthene, acenaphthofluoranthene, dibenzo[a,j]anthracene, dibenzo[a,h]anthracene, benzo[a]naphthalene, hexacene, naphtho[2,1-f]isoquinoline, α-naphthaphenanthridine, phenanthrooxazole, quinolino[6,5-f]quinoline, and benzothiophanthrene. These may have an alkyl group, an aryl group, an aromatic heterocyclic group, or a diarylamino group as a substituent.

Regarding the fluorescence-emitting dopant material, only one kind thereof may be contained in the light-emitting layer, or two or more kinds thereof may be contained. A content of the fluorescence-emitting dopant material is preferably 0.1 to 20 wt % and more preferably 1 to 10 wt % with respect to the host material.

The thermally activated delayed fluorescence-emitting dopant is not particularly limited. Examples thereof include: metal complexes such as a tin complex and a copper complex; indolocarbazole derivatives described in WO2011/070963A1; cyanobenzene derivatives and carbazole derivatives described in Nature 2012, 492, 234; and phenazine derivatives, oxadiazole derivatives, triazole derivatives, sulfone derivatives, phenoxazine derivatives, and acridine derivatives described in Nature Photonics 2014, 8,326.

The thermally activated delayed fluorescence-emitting dopant material is not particularly limited, and specific examples thereof include the following.

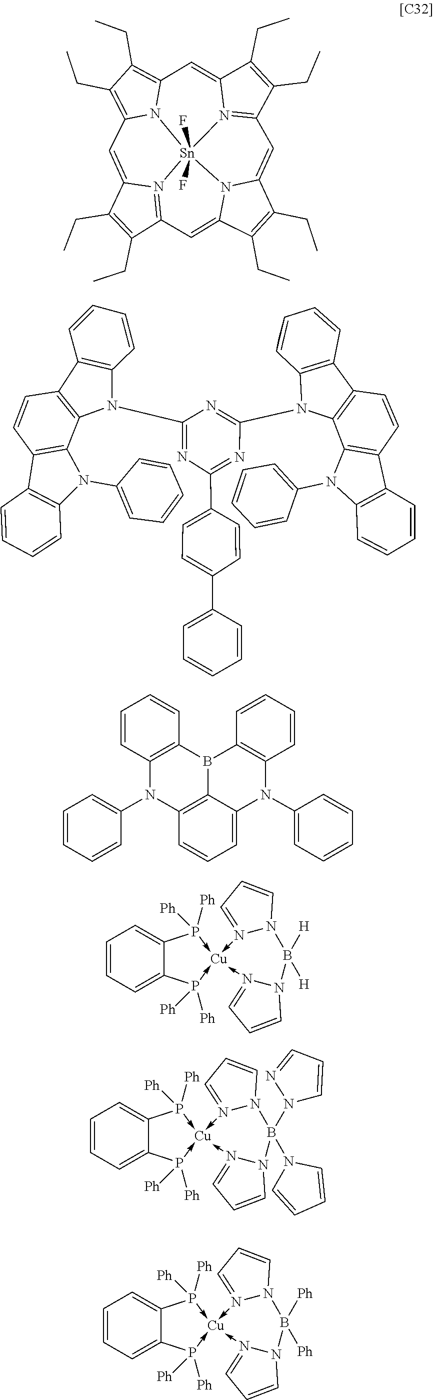

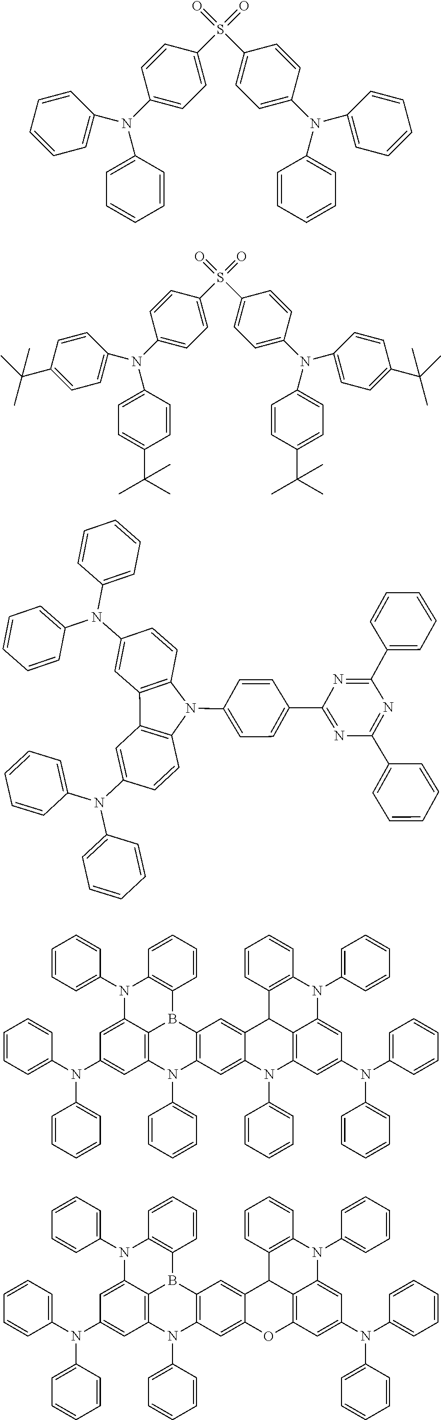

Regarding the thermally activated delayed fluorescence-emitting dopant material, only one kind thereof may be contained in the light-emitting layer, or two or more kinds thereof may be contained. In addition, the thermally activated delayed fluorescence-emitting dopant may be used by mixing with a phosphorescent dopant and a fluorescence-emitting dopant. A content of the thermally activated delayed fluorescence-emitting dopant material is preferably 0.1% to 50wt % and more preferably 1% to 30wt % with respect to the host material.

### Injection Layer

The injection layer is a layer that is provided between an electrode and an organic layer in order to lower a driving voltage and improve emission luminance, and includes a hole injection layer and an electron injection layer, and may be present between the anode and the light-emitting layer or the hole transport layer, and between the cathode and the light-emitting layer or the electron transport layer. The injection layer can be provided as necessary.

### Hole Blocking Layer

The hole blocking layer has a function of the electron transport layer in a broad sense, and is made of a hole blocking material having a function of transporting electrons and a significantly low ability to transport holes, and can block holes while transporting electrons, thereby improving a probability of recombining electrons and holes in the light-emitting layer.

### Electron Blocking Layer

The electron blocking layer has a function of a hole transport layer in a broad sense and blocks electrons while transporting holes, thereby enabling a probability of recombining electrons and holes in the light-emitting layer to be improved.

Regarding the material of the electron blocking layer, a known electron blocking layer material can be used and a material of the hole transport layer to be described below can be used as necessary. A film thickness of the electron blocking layer is preferably 3 to 100 nm, and more preferably 5 to 30 nm.

### Exciton Blocking Layer

The exciton blocking layer is a layer for preventing excitons generated by recombination of holes and electrons in the light-emitting layer from being diffused in a charge transport layer, and insertion of this layer allows excitons to be efficiently confined in the light-emitting layer, enabling the luminous efficiency of the device to be improved. The exciton blocking layer can be inserted, in a device having two or more light-emitting layers adjacent to each other, between two adjacent light-emitting layers.

Regarding the material of the exciton blocking layer, a known exciton blocking layer material can be used. Examples thereof include 1,3-dicarbazolyl benzene (mCP) and bis(2-methyl-8-quinolinolato)-4-phenylphenolato aluminum (III) (BAlq).

### Hole Transport Layer

The hole transport layer is made of a hole transport material having a function of transporting holes, and the hole transport layer can be provided as a single layer or a plurality of layers.

The hole transport material has either hole injection, transport properties or electron barrier properties, and may be an organic material or an inorganic material. For the hole transport layer, any one selected from conventionally known compounds can be used. Examples of such a hole transport material include porphyrin derivatives, arylamine derivatives, triazole derivatives, oxadiazole derivatives, imidazole derivatives, polyarylalkane derivatives, pyrazoline derivatives and pyrazolone derivatives, phenylenediamine derivatives, arylamine derivatives, amino-substituted chalcone derivatives, oxazole derivatives, styryl anthracene derivatives, fluorenone derivatives, hydrazone derivatives, stilbene derivatives, silazane derivatives, an aniline copolymer, and a conductive polymer oligomer, and particularly a thiophene oligomer. Use of porphyrin derivatives, arylamine derivatives, or styrylamine derivatives is preferred. Use of arylamine derivatives is more preferred.

### Electron Transport Layer

The electron transport layer is made of a material having a function of transporting electrons, and the electron transport layer can be provided as a single layer or a plurality of layers.

The electron transport material (which may also serve as a hole blocking material) may have a function of transferring electrons injected from the cathode to the light-emitting layer. For the electron transport layer, any one selected from conventionally known compounds can be used, and examples thereof include polycyclic aromatic derivatives such as naphthalene, anthracene, and phenanthroline, tris(8-quinolinolato)aluminum (III) derivatives, phosphine oxide derivatives, nitro-substituted fluorene derivatives, diphenylquinone derivatives, thiopyran dioxide derivatives, carbodiimide, fluorenylidene methane derivatives, anthraquinodimethane derivatives and anthrone derivatives, bipyridine derivatives, quinoline derivatives, oxadiazole derivatives, benzimidazole derivatives, benzothiazole derivatives, and indolocarbazole derivatives. In addition, a polymer material in which the above material is introduced into a polymer chain or the above material is used for a main chain of a polymer can be used.

The method for producing the organic EL device of the present invention comprises a step of mixing the first host material and the second host material in advance, and a step of vapor-depositing the resulting mixture from one vapor deposition source to form a light-emitting layer. Thus, two host materials can be mixed in advance, resulting in an enhancement in performance of an organic EL device. The mixing method here adopted can be powder mixing or melting and mixing.

A difference in temperature at 50% weight loss of the first host material and the second host material in the composition obtained by mixing in advance is preferably within 20° C.

The 50% weight reduction temperature is a temperature at which the weight is reduced by 50% when the temperature is raised from room temperature to 550° C. at a rate of 10° C./min in TG-DTA measurement under a nitrogen stream reduced pressure (1 Pa). It is considered that vaporization due to evaporation or sublimation the most vigorously occurs around this temperature.

### EXAMPLES

Hereafter, the present invention will be described in detail by referring to Examples, but the present invention is not limited these Examples and can be implemented in various forms without departing from the gist thereof.

Synthesis Examples of compound 011, compound 026, compound 027, compound 719-1, compound (g), and compound 719-2 are shown as representative examples. Other compounds were also synthesized by similar methods.

### Synthesis Example 1

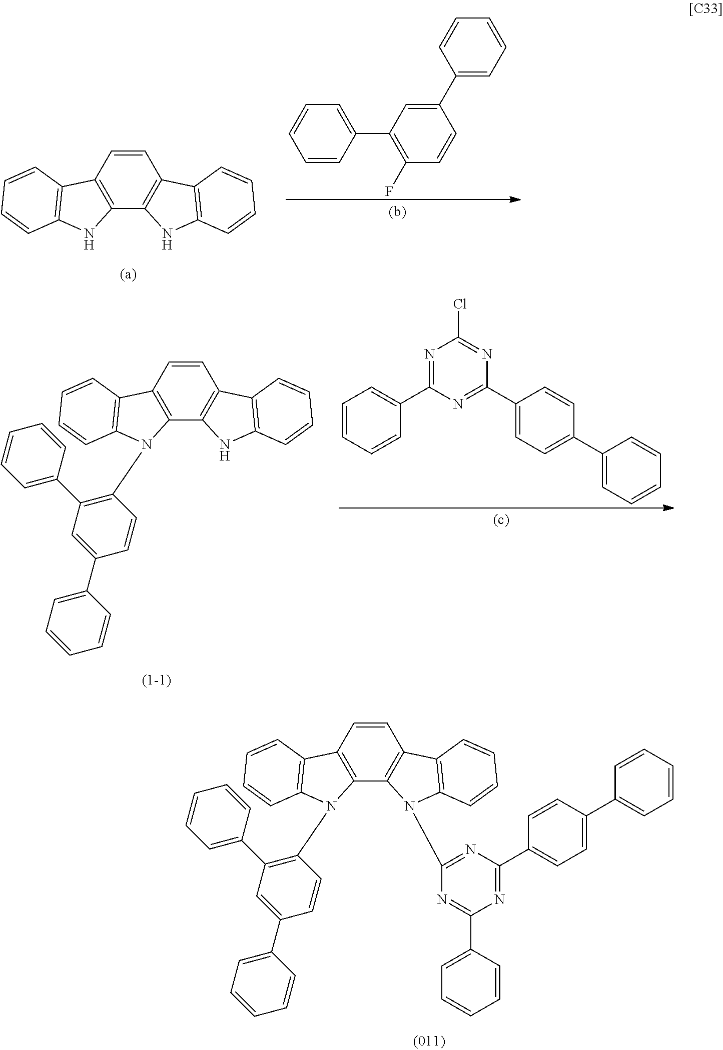

To 10 g of compound (a) were added 11 g of compound (b), 17 g of tripotassium phosphate, and 100 ml of 1,3-dimethyl-2-imidazolidinone, and the mixture was stirred under a nitrogen atmosphere at 200° C. for 48 hours. The mixture was cooled to room temperature, and then purified by silica gel column chromatography and crystallization to give 14.5 g (77% yield) of intermediate (1-1) as a white solid.

Under a nitrogen atmosphere, 2.7 g of 60 wt % sodium hydride was added to 30 ml of N,N′-dimethylacetamide to prepare a suspension. To this was added 10 g of intermediate (1-1) dissolved in 170 mL of N,N′ -dimethylacetamide, and the mixture was stirred for 30 minutes. To this was added 8.5 g of compound (c), then the mixture was stirred for 6 hours. The reaction solution was added to a solution of methanol (300 ml) mixed with distilled water (100 ml) while being stirred, and the resulting precipitated solid was collected by filtration. The resulting solid was purified by silica gel column chromatography and crystallization to give 12 g (73% yield) of compound 011 as a yellow solid (APCI-TOFMS, m/z 792 [M+H]⁺).

### Synthesis Example 2

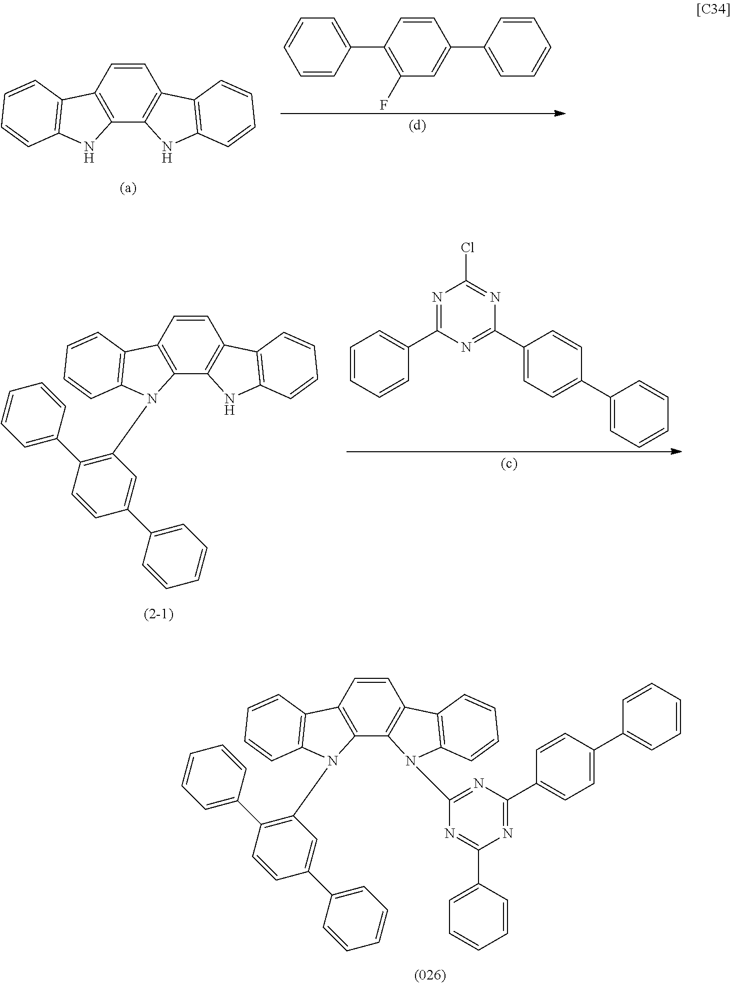

To 5 g of compound (a) were added 5.8 g of compound (d), 12.4 g of tripotassium phosphate, and 50 ml of 1,3-dimethyl-2-imidazolidinone, and the mixture was stirred under a nitrogen atmosphere at 200° C. for 48 hours. The mixture was cooled to room temperature, and then purified by silica gel column chromatography and crystallization to give 7.8 g (83% yield) of intermediate (2-1) as a white solid.

Under a nitrogen atmosphere, 1.3 g of 60 wt % sodium hydride was added to 20 ml of N,N′-dimethylacetamide to prepare a suspension. To this was added 5 g of intermediate (2-1) dissolved in 80 mL of N,N′-dimethylacetamide, and the mixture was stirred for 30 minutes. To this was added 3.9 g of compound (c), then the mixture was stirred for 6 hours. The reaction solution was added to a solution of methanol (200 ml) mixed with distilled water (50 ml) while being stirred, and the resulting precipitated solid was collected by filtration. The resulting solid was purified by silica gel column chromatography and crystallization to give 7.5 g (92% yield) of compound 026 as a yellow solid (APCI-TOFMS, m/z 792 [M+H]⁺).

### Synthesis Example 3

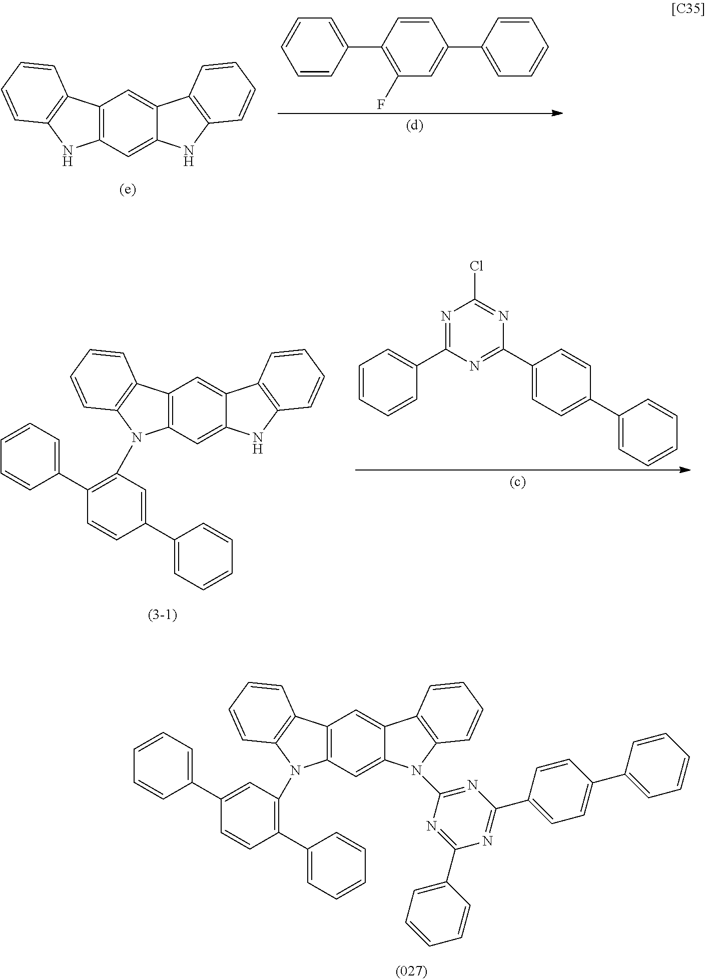

To 10 g of compound (e) were added 12 g of compound (d), 14.9 g of tripotassium phosphate, and 150 ml of 1,3-dimethyl-2-imidazolidinone, and the mixture was stirred under a nitrogen atmosphere at 200° C. for 48 hours. The mixture was cooled to room temperature, and then purified by silica gel column chromatography and crystallization to give 15.1 g (80% yield) of intermediate (3-1) as a white solid.

Under a nitrogen atmosphere, 2.6 g of 60 wt % sodium hydride was added to 30 ml of N,N′-dimethylacetamide to prepare a suspension. To this was added 10 g of intermediate (3-1) dissolved in 170 mL of N,N′-dimethylacetamide, and the mixture was stirred for 30 minutes. To this was added 7.8 g of compound (c), then the mixture was stirred for 6 hours. The reaction solution was added to a solution of methanol (200 ml) mixed with distilled water (50 ml) while being stirred, and the resulting precipitated solid was collected by filtration. The resulting solid was purified by silica gel column chromatography and crystallization to give 12.1 g (74% yield) of compound 027 as a yellow solid (APCI-TOFMS, m/z 792 [M+H]⁺).

### Synthesis Example 4

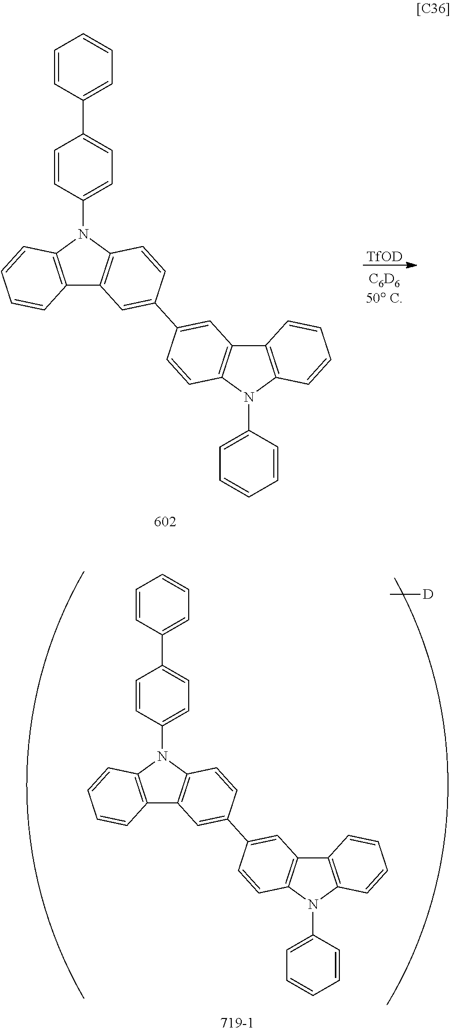

To 8.3 g of compound 602 were added 160 ml of deuterated benzene (C₆D₆) and 10.0 g of deuterated trifluoromethanesulfonic acid (TfOD), and the mixture was heated and stirred under a nitrogen atmosphere at 50° C. for 6.5 hours. The reaction liquid was added to a solution of sodium carbonate (7.4 g) in deuterated water (200 ml), and the mixture was rapidly cooled, and subjected to separation and purification to give 2.5 g of compound 719-1 as a deuterated product of a white solid.

The rate of deuteration of compound 719-1 was determined by proton nuclear magnetic resonance spectroscopy. A measurement sample was prepared by dissolving compound 719-1 (5.0 mg) and dimethylsulfone (2.0 mg) as an internal standard material in deuterated tetrahydrofuran (1.0 ml). The average proton concentration [mol/g] in compound 719-1 included in the measurement sample was calculated from the ratio between the respective integral intensities derived from the internal standard material and compound 719-1. The average proton concentration [mol/g] in a non-deuterated product (corresponding to compound 602) of compound 719-1 was also calculated in the same manner. Next, the ratio between the proton concentration in compound 719-1 and the proton concentration in compound 602 was calculated, and subtracted from 1, and thus the rate of deuteration on average of compound 719-1 was calculated. The results are shown in Table 1.

### Synthesis Example 5

Compound (g) was synthesized according to the following reaction.

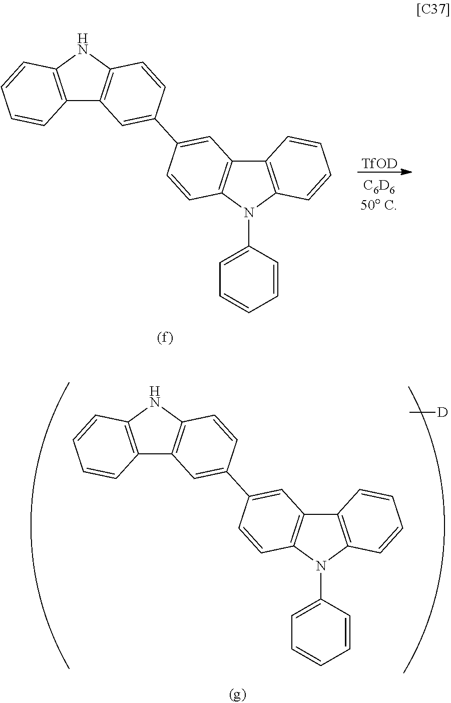

To 10.0 g of compound (f) were added 240 ml of deuterated benzene (C₆D₆) and 18.4 g of deuterated trifluoromethanesulfonic acid (TfOD), and the mixture was heated and stirred under a nitrogen atmosphere at 50° C. for 5.0 hours. The reaction liquid was added to a solution of sodium carbonate (14.3 g) in deuterated water (150 ml), and the mixture was rapidly cooled, and subjected to separation and purification to give 8.9 g of compound (g) as a deuterated product.

### Synthesis Example 6

Compound 719-2 was synthesized in accordance with the following reaction.

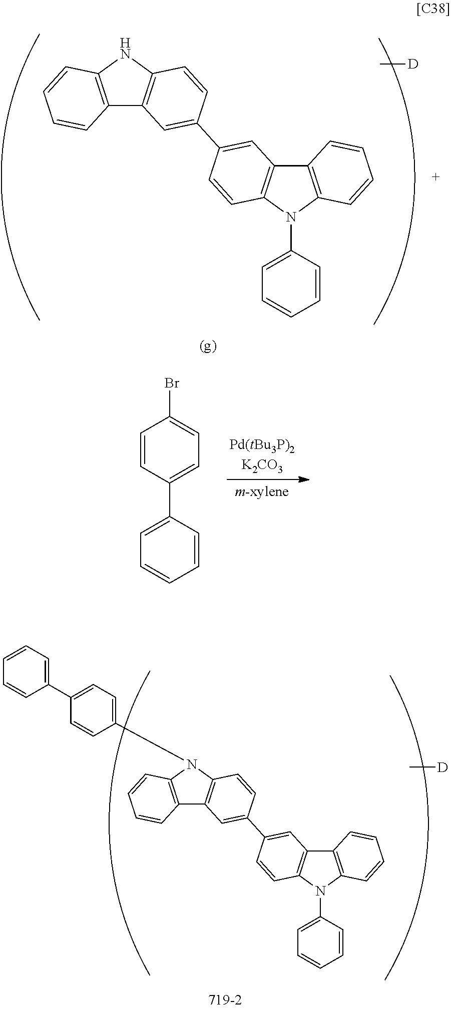

To compound (g) (5.0 g) were added 4.3 g of p-bromophenyl, 100 ml of m-xylene, 0.4 g of bis(tri-tert-butylphosphine) palladium, and 5.0 g of potassium carbonate, and the mixture was stirred under a nitrogen atmosphere with heating and flux for 5 hours. The reaction liquid was cooled, and then subjected to separation and purification to give 2.7 g of compound 719-2 as a white solid compound being a deuterated product.

The rate of deuteration of 719-2 was calculated in the same manner as in 719-1. The results are shown in Table 1.

TABLE 1

| | | Rate of |
|---|---|---|
| Compound | | deuteration |
| Synthesis | 719-1 | 81% |
| Example 7 | | |
| Synthesis | 719-2 | 65% |
| Example 9 | | |

Compounds used in Examples and Comparative Examples are shown below.

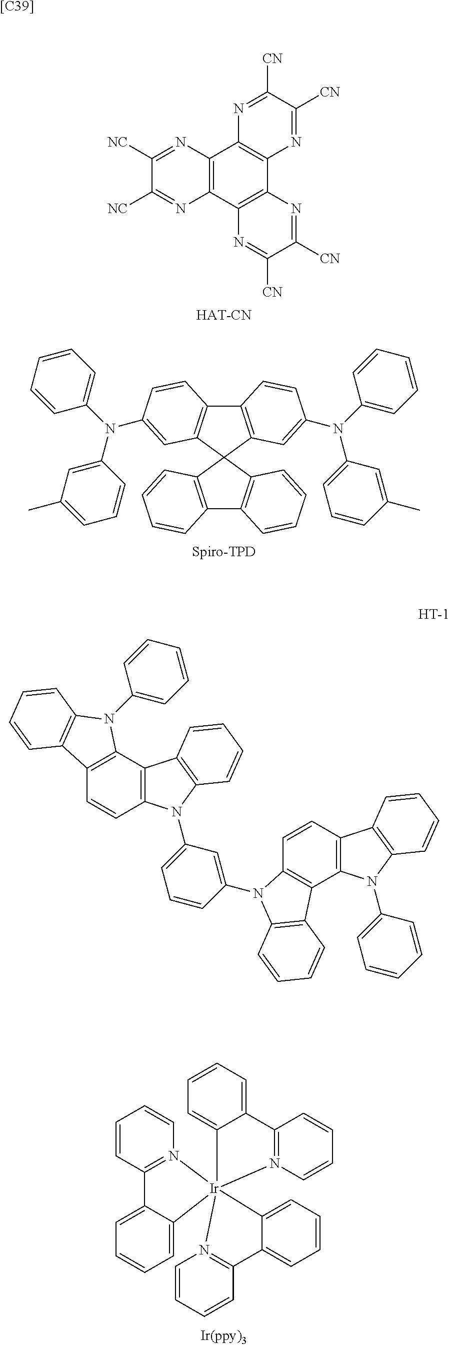

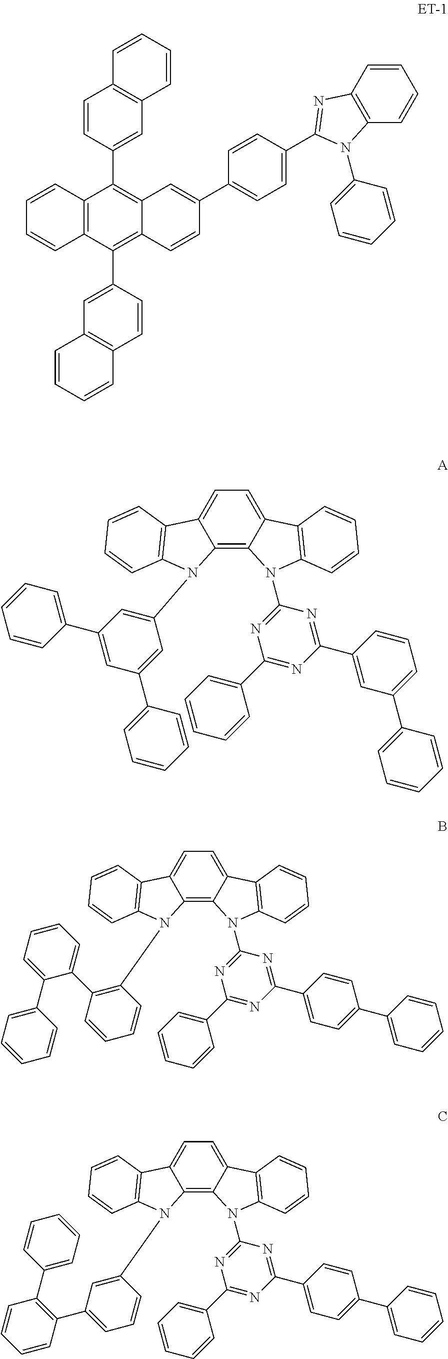 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2cccc(-c3nc(-c4ccccc4)nc(-n4c5ccccc5c5ccc6c7ccccc7n(-c7cc(-c8ccccc8)cc(-c8ccccc8)c7)c6c54)n3)c2)cc1 ; c1ccc(-c2ccc(-c3nc(-c4ccccc4)nc(-n4c5ccccc5c5ccc6c7ccccc7n(-c7ccccc7-c7ccccc7-c7ccccc7)c6c54)n3)cc2)cc1 ; c1ccc(-c2ccc(-c3nc(-c4ccccc4)nc(-n4c5ccccc5c5ccc6c7ccccc7n(-c7cccc(-c8ccccc8-c8ccccc8)c7)c6c54)n3)cc2)cc1]

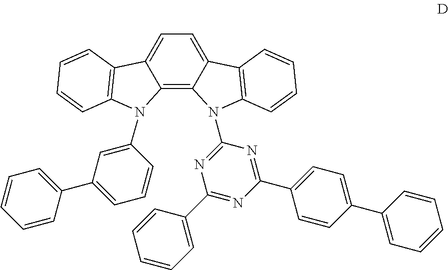 [SMILES: c1ccc(-c2ccc(-c3nc(-c4ccccc4)nc(-n4c5ccccc5c5ccc6c7ccccc7n(-c7cccc(-c8ccccc8)c7)c6c54)n3)cc2)cc1]

### Example 1

On a glass substrate on which an anode made of ITO with a film thickness of 70 nm was formed, respective thin films were laminated by a vacuum evaporation method at a degree of vacuum of 4.0×10⁻⁵ Pa. First, HAT-CN was formed with a thickness of 25 nm as a hole injection layer on ITO, and next, Spiro-TPD was formed with a thickness of 30 nm as a hole transport layer. Next, HT-1 was formed with a thickness of 10 nm as an electron blocking layer. Then, compound 006 as a host and Ir(ppy)₃ as a light-emitting dopant were co-vapor-deposited from different vapor deposition sources, respectively, to form a light-emitting layer with a thickness of 40 nm. In this case, co-vapor deposition was performed under vapor deposition conditions such that the concentration of Ir(ppy)₃ was 10 wt %. Next, ET-1 was formed with a thickness of 20 nm as an electron transport layer. Further, LiF was formed with a thickness of 1 nm as an electron injection layer on the electron transport layer. Finally, Al was formed with a thickness of 70 nm as a cathode on the electron injection layer to produce an organic EL device.

### Examples 2 to 3 and Comparative Examples 1 to 2

Organic EL devices were produced in the same manner as in Example 1 except that compounds shown in Table 2 were used as the hosts in Example 1.

Evaluation results of the produced organic EL devices are shown in Table 2. In the table, the luminance, voltage, and power efficiency are values at a driving current of 20 mA/cm², and they exhibit initial characteristics. LT70 is a time period needed for the luminance to be reduced to 70% of the initial luminance that is assumed to be 100% at a driving current of 20 mA/cm², and it represents lifetime characteristics. The numbers with which the host compound, the first host, and the second host are marked are numbers with which the exemplified compounds are marked.

TABLE 2

| | | | | Power | |
|---|---|---|---|---|---|
| | Host | Luminance | Voltage | efficiency | LT70 |
| | compound | (cd/m²) | (V) | (lm/W) | (h) |
| Example 1 | 006 | 3.4 | 10843 | 49.7 | 659 |
| Example 2 | 046 | 3.4 | 10936 | 50.1 | 526 |
| Example 3 | 327 | 3.5 | 11222 | 49.9 | 479 |
| Comp. | A | 3.6 | 10545 | 46.1 | 329 |
| Example 1 | | | | | |
| Comp. | B | 3.6 | 9069 | 39.9 | 110 |
| Example 2 | | | | | |

### Example 4

On a glass substrate on which an anode made of ITO with a film thickness of 110 nm was formed, respective thin films were laminated by a vacuum evaporation method at a degree of vacuum of 4.0×10⁻⁵ Pa. First, HAT-CN was formed with a thickness of 25 nm as a hole injection layer on ITO, and next, Spiro-TPD was formed with a thickness of 30 nm as a hole transport layer. Next, HT-1 was formed with a thickness of 10 nm as an electron blocking layer. Next, as shown in Table 3, compound 011 as a first host, compound 602 as a second host and Ir(ppy)₃ as a light-emitting dopant were co-vapor-deposited from different vapor deposition sources, respectively, to form a light-emitting layer with a thickness of 40 nm. In this case, co-vapor deposition was performed under vapor deposition conditions such that the concentration of Ir(ppy)₃ was 10 wt %, and the weight ratio between the first host and the second host was 30:70. Next, ET-1 was formed with a thickness of 20 nm as an electron transport layer. Further, LiF was formed with a thickness of 1 nm as an electron injection layer on the electron transport layer. Finally, Al was formed with a thickness of 70 nm as a cathode on the electron injection layer to produce an organic EL device.

### Examples 5 to 14

Organic EL devices were produced in the same manner as in Example 4 except that compounds shown in Table 3 were used as the first host and the second host and the weight ratio was as shown in Table 3.

### Examples 15 to 22

Organic EL devices were produced in the same manner as in Example 4 except that a premixture obtained by weighing a first host and a second host shown in Table 3 at a weight ratio shown in Table 3 and mixing them while grinding in a mortar was vapor-deposited from one vapor deposition source.

### Comparative Examples 3 to 8

Organic EL devices were produced in the same manner as in Example 4 except that compounds shown in Table 3 were used as the first host and the second host and the weight ratio was as shown in Table 3.

### Comparative Examples 9 and 10

Organic EL devices were produced in the same manner as in Example 4 except that a premixture obtained by weighing a first host and a second host shown in Table 3 at a weight ratio shown in Table 3 and mixing them while grinding in a mortar was vapor-deposited from one vapor deposition source.

Evaluation results of the produced organic EL devices are shown in Table 3. In the table, the luminance, voltage, and power efficiency are values at a driving current of 20 mA/cm², and they exhibit initial characteristics. LT70 is a time period needed for the luminance to be reduced to 70% of the initial luminance that is assumed to be 100% at a driving current of 20 mA/cm², and it represents lifetime characteristics. The weight ratio corresponds to first host:second host.

TABLE 3

| | | | | | | Power | |
|---|---|---|---|---|---|---|---|
| | First | Second | Weight | Voltage | Luminance | efficiency | LT70 |
| | host | host | ratio | (V) | (cd/m²) | (lm/W) | (h) |
| Example 4 | 011 | 602 | 30:70 | 4.2 | 9421 | 35.4 | 2049 |
| Example 5 | 006 | 602 | 30:70 | 4.6 | 10410 | 35.8 | 1924 |
| Example 6 | 022 | 602 | 30:70 | 4.5 | 9951 | 34.5 | 1772 |
| Example 7 | 026 | 602 | 30:70 | 4.3 | 10133 | 36.7 | 2075 |
| Example 8 | 027 | 602 | 30:70 | 4.4 | 9587 | 34.3 | 1943 |
| Example 9 | 046 | 602 | 30:70 | 4.4 | 10512 | 37.5 | 1875 |
| Example 10 | 051 | 602 | 30:70 | 4.5 | 10409 | 36.6 | 1921 |
| Example 11 | 056 | 602 | 30:70 | 4.3 | 10156 | 37.2 | 1979 |
| Example 12 | 152 | 602 | 30:70 | 4.0 | 10080 | 39.4 | 1691 |
| Example 13 | 152 | 643 | 50:50 | 3.8 | 9571 | 39.2 | 1337 |
| Example 14 | 026 | 602 | 50:50 | 4.1 | 9809 | 37.8 | 1440 |
| Example 15 | 026 | 602 | 50:50 | 3.9 | 9996 | 39.9 | 1643 |
| Example 16 | 026 | 602 | 30:70 | 4.2 | 10318 | 38.1 | 2256 |
| Example 17 | 006 | 602 | 30:70 | 4.5 | 10106 | 35.5 | 2044 |
| Example 18 | 006 | 602 | 50:50 | 4.2 | 10198 | 38.3 | 1587 |
| Example 19 | 006 | 719-1 | 30:70 | 4.5 | 10423 | 36.4 | 2504 |
| Example 20 | 006 | 719-2 | 30:70 | 4.5 | 10397 | 36.2 | 2259 |
| Example 21 | 046 | 719-1 | 30:70 | 4.5 | 10474 | 36.2 | 2428 |
| Example 22 | 046 | 719-2 | 30:70 | 4.6 | 10461 | 35.9 | 2192 |
| Comp. | A | 602 | 30:70 | 4.6 | 9817 | 33.2 | 1581 |
| Example 3 | | | | | | | |
| Comp. | B | 602 | 30:70 | 4.6 | 9490 | 32.7 | 588 |
| Example 4 | | | | | | | |
| Comp. | C | 602 | 30:70 | 4.6 | 9461 | 32.3 | 421 |
| Example 5 | | | | | | | |
| Comp. | A | 643 | 30:70 | 4.6 | 9523 | 32.2 | 1075 |
| Example 6 | | | | | | | |
| Comp. | D | 602 | 30:70 | 4.5 | 9905 | 34.3 | 1914 |
| Example 7 | | | | | | | |
| Comp. | A | 643 | 50:50 | 4.2 | 9519 | 35.5 | 710 |
| Example 8 | | | | | | | |
| Comp. | A | 643 | 50:50 | 4.2 | 9713 | 36.6 | 733 |
| Example 9 | | | | | | | |
| Comp. | A | 643 | 30:70 | 4.5 | 9818 | 34.4 | 1102 |
| Example 10 | | | | | | | |

From the results in Table 2 and Table 3, it is understood that Examples 1 to 22 improved the power efficiency and the lifetime and exhibited good characteristics, as compared with Comparative Examples.

Table 4 shows the temperature at 50% weight loss (T₅₀) of compounds 006, 046, 026, 602, 643, 719-1, and 719-2, and compound A.

TABLE 4

| Compound | T₅₀[° C.] |
|---|---|
| 006 | 273 |
| 046 | 276 |
| 026 | 275 |
| 602 | 277 |
| 643 | 291 |
| 719-1 | 278 |
| 719-2 | 277 |
| A | 288 |

1. A host material for an organic electroluminescent device, represented by any of the following general formulas (1) to (5): 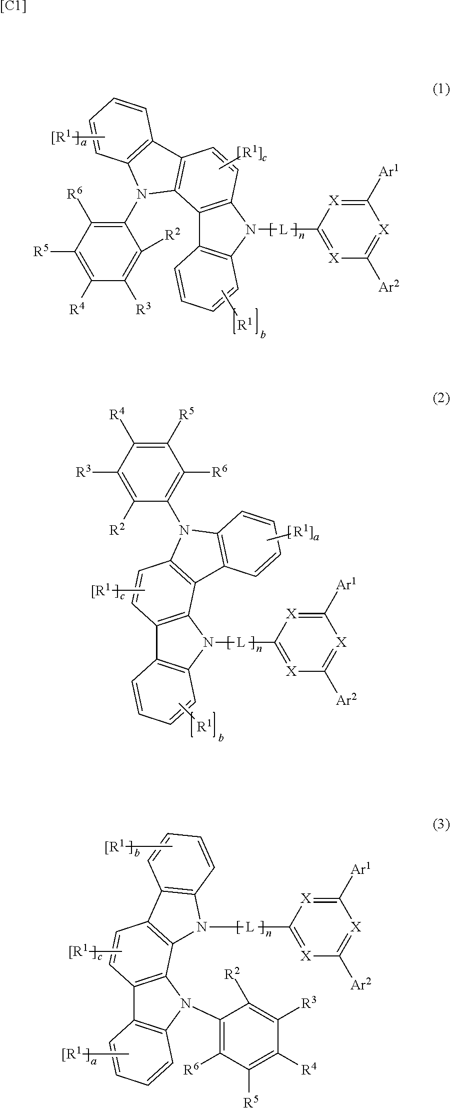 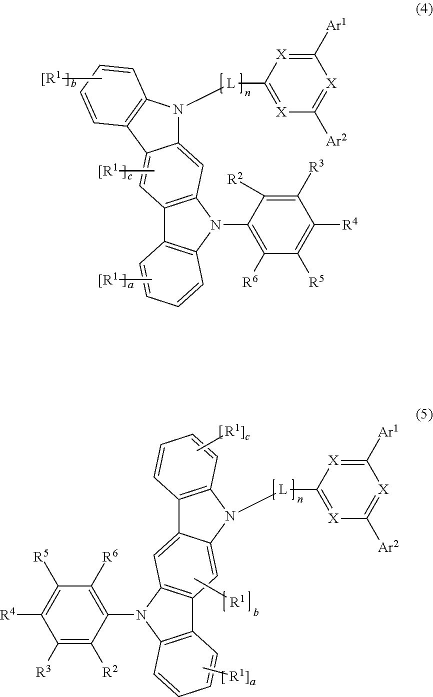
wherein, in the formulas (1) to (5), each X independently represents N or C—H and at least one thereof represents N,
L independently represents a substituted or unsubstituted aromatic hydrocarbon group having 6 to 30 carbon atoms,
Ar¹ and Ar² each independently represent hydrogen, a substituted or unsubstituted aromatic hydrocarbon group having 6 to 30 carbon atoms, a substituted or unsubstituted aromatic heterocyclic group having 3 to 18 carbon atoms, or a substituted or unsubstituted linked aromatic group in which two to five aromatic rings of such an aromatic hydrocarbon group or aromatic heterocyclic group are linked to each other, each R¹ independently represents an aliphatic hydrocarbon group having 1 to 10 carbon atoms, a substituted or unsubstituted aromatic hydrocarbon group having 6 to 30 carbon atoms, or a substituted or unsubstituted aromatic heterocyclic group having 3 to 18 carbon atoms,
R² represents an aliphatic hydrocarbon group having 1 to 10 carbon atoms, a substituted or unsubstituted aromatic hydrocarbon group having 6 to 30 carbon atoms, a substituted or unsubstituted aromatic heterocyclic group having 3 to 18 carbon atoms, or a substituted or unsubstituted linked aromatic group in which two to five aromatic rings of such an aromatic hydrocarbon group or aromatic heterocyclic group are linked to each other,
R³ to R⁶ each independently represent hydrogen, an aliphatic hydrocarbon group having 1 to 10 carbon atoms, a substituted or unsubstituted aromatic hydrocarbon group having 6 to 30 carbon atoms, a substituted or unsubstituted aromatic heterocyclic group having 3 to 18 carbon atoms, or a substituted or unsubstituted linked aromatic group in which two to five aromatic rings of such an aromatic hydrocarbon group or aromatic heterocyclic group are linked to each other, and at least one of R³ to R⁶ represents a substituted or unsubstituted aromatic hydrocarbon group having 6 to 30 carbon atoms, or a substituted or unsubstituted aromatic heterocyclic group having 3 to 18 carbon atoms, and
a to c represent the number of substitutions, a and b represent an integer of 0 to 4, and c represents an integer of 0 to 2, and n represents the number of repetitions and an integer of 0 to 3.

2. The host material according to claim 1, wherein L represents a substituted or unsubstituted phenylene group and n represents 1 or 2 in the general formulas (1) to (5).

3. The host material according to claim 1, wherein n in the general formulas (1) to (5) represents 0.

4. The host material according to claim 3, wherein the general formulas (1) to (5) are represented by any of the following formulas (6) to (9): 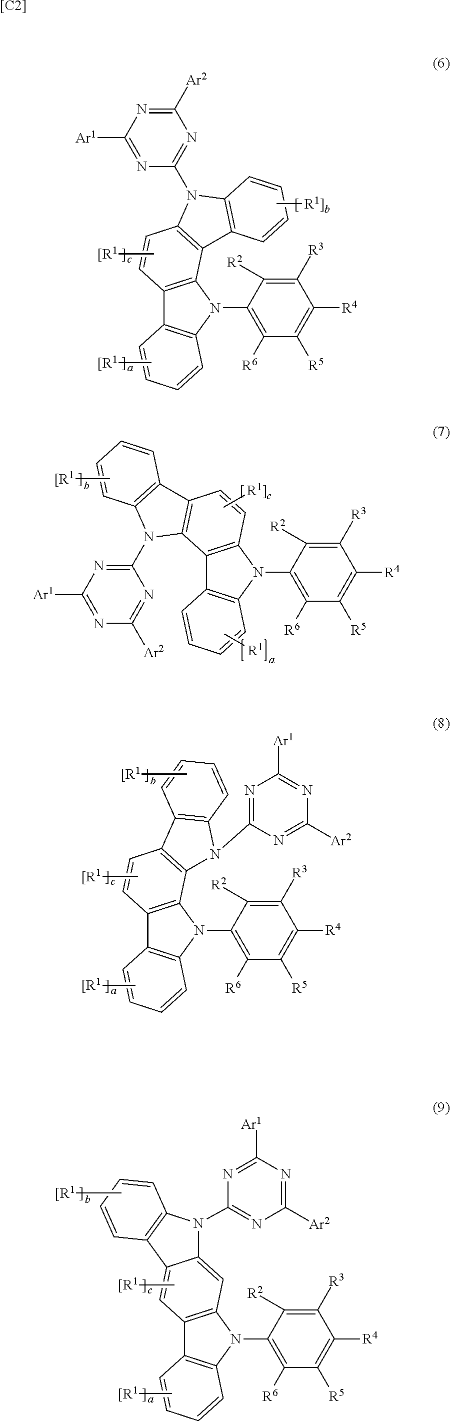
wherein, in the formulas (6) to (9), Ar¹, Ar², and a to c are as defined for the general formulas (1) to (5),
each R¹ independently represents a substituted or unsubstituted aromatic hydrocarbon group having 6 to 18 carbon atoms, or a substituted or unsubstituted aromatic heterocyclic group having 3 to 12 carbon atoms,
R² represents a substituted or unsubstituted aromatic hydrocarbon group having 6 to 18 carbon atoms, a substituted or unsubstituted aromatic heterocyclic group having 3 to 12 carbon atoms, or a substituted or unsubstituted linked aromatic group in which two to five of these aromatic rings are linked to each other, and
R³ to R⁶ each independently represent hydrogen, a substituted or unsubstituted aromatic hydrocarbon group having 6 to 18 carbon atoms, a substituted or unsubstituted aromatic heterocyclic group having 3 to 12 carbon atoms, or a substituted or unsubstituted linked aromatic group in which two to five aromatic rings of such an aromatic hydrocarbon group or aromatic heterocyclic group are linked to each other, and at least one of R³ to R⁶ represents a substituted or unsubstituted aromatic hydrocarbon group having 6 to 18 carbon atoms, or a substituted or unsubstituted aromatic heterocyclic group having 3 to 12 carbon atoms.

5. An organic electroluminescent device comprising one or more light-emitting layers between an anode and a cathode opposed to each other, wherein at least one of the light-emitting layers contains a first host material selected from the host material according to claim 1, a second host material selected from a compound represented by the following general formula (10), and a light-emitting dopant material: 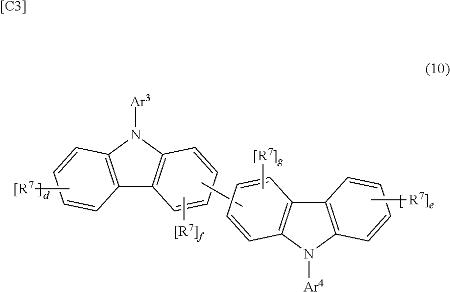
wherein Ar³ and Ar⁴ each independently represent a substituted or unsubstituted aromatic hydrocarbon group having 6 to 18 carbon atoms, a substituted or unsubstituted aromatic heterocyclic group having 3 to 17 carbon atoms, or a substituted or unsubstituted linked aromatic group in which two to five of these aromatic rings are linked to each other,
each R⁷ independently represents deuterium, an aliphatic hydrocarbon group having 1 to 10 carbon atoms, a substituted or unsubstituted aromatic hydrocarbon group having 6 to 18 carbon atoms, or a substituted or unsubstituted aromatic heterocyclic group having 3 to 17 carbon atoms, and
d to g represent the number of substitutions, d and e represent an integer of 0 to 4, and f and g represent an integer of 0 to 3.

6. The organic electroluminescent device according to claim 5, wherein Ar³ and Ar⁴ each independently represent a substituted or unsubstituted phenyl group, a substituted or unsubstituted biphenyl group, or a substituted or unsubstituted terphenyl group.

7. The organic electroluminescent device according to claim 5, wherein the first host material is a host material in which all of a to c in the general formulas (1) to (5) or formulas (6) to (9) represent 0, and the second host material is a host material in which all of d to g in the general formula (10) represent 0.

8. The organic electroluminescent device according to claim 5, wherein the light-emitting dopant material is an organic metal complex containing at least one metal selected from ruthenium, rhodium, palladium, silver, rhenium, osmium, iridium, platinum and gold.

9. The organic electroluminescent device according to claim 5, wherein the light-emitting dopant material is a thermally activated delayed fluorescence-emitting dopant material.

10. A method for producing the organic electroluminescent device according to claim 5, comprising a step of mixing the first host material and the second host material in advance, and a step of vapor-depositing the resulting mixture from one vapor deposition source to form a light-emitting layer.

11. A composition comprising a first host material selected from the host material according to claim 1, and a second host material selected from a compound represented by the following general formula (10): 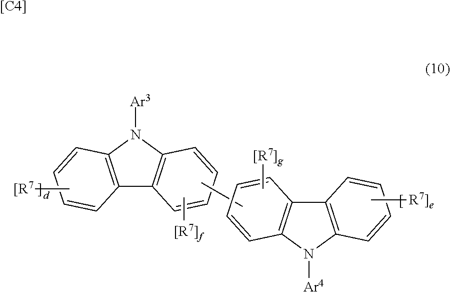
wherein Ar³ and Ar⁴ each independently represent a substituted or unsubstituted aromatic hydrocarbon group having 6 to 18 carbon atoms, a substituted or unsubstituted aromatic heterocyclic group having 3 to 17 carbon atoms, or a substituted or unsubstituted linked aromatic group in which two to five of these aromatic rings are linked to each other,
each R⁷ independently represents deuterium, an aliphatic hydrocarbon group having 1 to 10 carbon atoms, a substituted or unsubstituted aromatic hydrocarbon group having 6 to 18 carbon atoms, or a substituted or unsubstituted aromatic heterocyclic group having 3 to 17 carbon atoms, and
d to g represent the number of substitutions, d and e represent an integer of 0 to 4, and f and g represent an integer of 0 to 3.

12. The composition according to claim 11, wherein the first host material is a host material in which all of a to c in the general formulas (1) to (5) or formulas (6) to (9) represent 0, and the second host material is a host material in which all of d to g in the general formula (10) represent 0.

13. The composition according to claim 11, wherein a difference in temperature at 50% weight loss of the first host material and the second host material is within 20° C.
